(19) United States
(12) Patent Application Publication
(10) Pub. No.: US 2025/0177737 A1
(43) Pub. Date: Jun. 5, 2025

(54) TRANSIENT CLOSED-LOOP SYSTEM AND APPLICATIONS OF SAME

(71) Applicants: NORTHWESTERN UNIVERSITY, Evanston, IL (US); THE GEORGE WASHINGTON UNIVERSITY, Washington, DC (US)

(72) Inventors: Yeonsik Choi, Evanston, IL (US); Hyoyoung Jeong, Evanston, IL (US); John A. Rogers, Wilmette, IL (US); Rose T. Yin, Washington, DC (US); Igor Efimov, Washington, DC (US)

(21) Appl. No.: 18/845,029

(22) PCT Filed: Mar. 16, 2023
(86) PCT No.: PCT/US2023/064507

§ 371 Date: Sep. 9, 2024

Related U.S. Application Data
(63) Continuation-in-part of application No. PCT/US2022/035089, filed on Jun. 27, 2022.
(60) Provisional application No. 63/320,790, filed on Mar. 17, 2022.
(60) Provisional application No. 63/215,070, filed on Jun. 25, 2021.

(51) Int. Cl. A61N 1/05; A61N 1/365; A61N 1/37; A61N 1/39; G16H 40/40

(52) U.S. Cl. CPC A61N 1/0587; A61N 1/365; A61N 1/37; A61N 1/3956; G16H 40/40

(57) ABSTRACT

A transient closed-loop system for cardiac pacing and/or defibrillator therapy for a subject includes a bioresorbable module configured to at least partially attach to an epicardial interface of the subject's heart for the cardiac pacing; at least one skin-interfaced module configured to attach to an outer surface of the subject's skin, wherein the bioresorbable module is in wireless communication with the at least one skin-interfaced module; and a control module in wireless communication with the at least one skin-interfaced module.

### CROSS-REFERENCE TO RELATED PATENT APPLICATIONS

This application claims priority to and the benefit of U.S. Provisional Application No. 63/320,790, filed Mar. 17, 2022, which is incorporated herein in its entirety by reference.

This application is also a continuation in part application of PCT Patent Application No. PCT/US2022/035089, filed Jun. 27, 2022, which itself claims priority to and the benefit of U.S. Provisional Patent Application No. 63/215,070, filed Jun. 25, 2021, which are incorporated herein in their entireties by reference.

### STATEMENT AS TO RIGHTS UNDER FEDERALLY-SPONSORED RESEARCH

This invention was made with government support under grant numbers HL155844 and HL141470 awarded by the National Institutes of Health. The government has certain rights in the invention.

### FIELD OF THE INVENTION

The invention relates generally to biosensors, and more particularly to a transient closed-loop system and applications of the same.

### BACKGROUND OF THE INVENTION

The background description provided herein is for the purpose of generally presenting the context of the invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions. Work of the presently named inventors, to the extent it is described in the background of the invention section, as well as aspects of the description that may not otherwise qualify as prior art at the time of filing, are neither expressly nor impliedly admitted as prior art against the invention.

All living systems function through the interaction of complex networks of physiological feedback loops to maintain homeostasis. Engineering approaches to treat disorders, such as those based on cardiac pacemakers, exploit conceptually similar methods for closed-loop control to enable autonomous, adaptive regulation of one or more essential physiological parameters to target set points, without human intervention. These and other existing platforms have key limitations that follow from their reliance on conventional electronic hardware, monitoring schemes and interfaces to the body. First, these systems often require physical tethers and percutaneous access points that may lead to systemic infections. Second, connections to external modules for power supply, sensing, control and other essential functions constrain patient mobility and impede clinical care. Third, removal or replacement of electronic components (e.g., leads and batteries) demands surgical procedures that impose additional burdens on patients and the healthcare system. These features can extend durations of hospitalization, often in intensive care units.

For example, arrhythmias are common complications after cardiac surgery and represent a major source of morbidity and mortality. Bradyarrhythmias are particularly common after valve surgery and are a consequence of direct surgical injury and local edema. After valve surgery and/or coronary artery bypass graft (CABG) surgery, bradycardia usually is caused by sinus node dysfunction or atrioventricular conduction disturbances. Short-term bradyarrhythmias that commonly occur in the 5-7 days after cardiac surgeries must be treated with temporary percutaneous pacing systems, typically prolonging hospital stays with limited ability to initiate physical therapy. In these cases, temporary pacemakers are implanted to treat the transient arrhythmias. Temporary pacemakers may also act as a bridge to therapy, as a permanent pacemaker is implanted if symptomatic bradyarrhythmias, such as atrioventricular block and sick sinus syndrome, persist longer than 5-7 days postoperatively. If the underlying intrinsic rhythm is absent or temporary pacing leads fail, permanent pacing may be performed earlier. Removal of the leads can cause laceration and perforation of the myocardium, dislodging of blood clots in the lungs, or eventual loss of vascular access. Recently reported wireless, bioresorbable electronic implants for temporary therapies address some of these challenges, however, they still require external, wall-plugged equipment for monitoring, power and control, and thus patient's health, mobility, and comfort are still impaired significantly.

Therefore, a heretofore unaddressed need exists in the art to address the aforementioned deficiencies and inadequacies.

### SUMMARY OF THE INVENTION

In light of the foregoing, this invention discloses a transient closed-loop system that combines a time-synchronized, wireless network of soft, skin-integrated devices with an advanced bioresorbable pacemaker to control cardiac rhythms, track cardiopulmonary status, provide multi-haptic feedback, and enable transient operation with minimal patient burden. This system provides a range of robust, rate-adaptive cardiac pacing capabilities, as demonstrated in rat, canine, and human heart studies of autonomous treatment of bradycardias. This work establishes a generalizable engineering framework for closed-loop temporary electrotherapy using wirelessly linked, body-integrated bioelectronic devices.

In one aspect of the invention, the transient closed-loop system for cardiac pacing and/or defibrillator therapy for a subject, comprises a bioresorbable module configured to at least partially attach to an epicardial interface of the subject's heart for the cardiac pacing; at least one skin-interfaced module configured to attach to an outer surface of the subject's skin, wherein the bioresorbable module is in wireless communication with the at least one skin-interfaced module; and a control module in wireless communication with the at least one skin-interfaced module.

In one embodiment, the bioresorbable module dissolves in the subject's body after a period of time.

In one embodiment, the period of time is at least 10 days, 20 days or 30 days.

In one embodiment, the period of time is customizable.

In one embodiment, the control module is configured to calculate at least one regular heart rate and provide autonomous cardiac pacing to the subject according to the at least one regular heart rate.

In one embodiment, the at least one regular heart rate comprises a high rate limit and a low rate limit.

In one embodiment, when a heart rate of the subject is lower than the low rate limit, the control module activates the bioresorbable module to provide electrical stimulation to the heart at a pre-specified rate, for cardiac pacing.

In one embodiment, when the heart rate of the subject is higher than the high rate limit, the bioresorbable module remains inactive.

In one embodiment, the at least one skin-interfaced module comprises a cardiac module.

In one embodiment, the cardiac module is configured to operably place over skin of the subject's chest area.

In one embodiment, the at least one skin-interfaced module further comprises a respiration module in wireless communication with the control module.

In one embodiment, the respiration module is configured to operably collect physiological information of the subject and wirelessly transmit the physiological information to the control module.

In one embodiment, the control module operably calculates the at least one regular heart rate according to the physiological information collected by the respiration module and provides autonomous cardiac pacing to the subject according to the at least one regular heart rate.

In one embodiment, the at least one skin-interfaced module further comprises a hemodynamic module in wireless communication with the control module.

In one embodiment, the hemodynamic module is configured to operably collect physiological information of the subject and wirelessly transmit the physiological information to the control module.

In one embodiment, the control module operably calculates the at least one regular heart rate according to the physiological information collected by the hemodynamic module and provides autonomous cardiac pacing to the subject according to the at least one regular heart rate.

In one embodiment, the at least one skin-interfaced module further comprises a haptic module in wireless communication with the control module.

In one embodiment, the haptic module in configured to operably receive tactile information from the control module.

In one embodiment, the haptic module operably provides at least one pattern of vibro-tactile according to the tactile information received from the control module.

In one embodiment, the bioresorbable module comprises a power harvester configured to operably receive power delivery from the cardiac module; at least one stimulation electrode configured to operably deliver stimuli to the epicardial interface of the subject's heart for the cardiac pacing; and a stretchable interconnect connecting the power harvester and the stimulation electrode.

In one embodiment, the bioresorbable module is encapsulated by bioresorbable dynamic covalent polyurethane (b-DCPU), polylactic acid (PLA), polyglycolic acid (PGA), polyglycolide (PGL), polycaprolactone (PCL), poly(glycerol sebacate) (PGS), poly(octamethylenemaleate (anhydride) citrate)) (POMaC), poly(1,8-octanediol-co-citric acid) (POC), poly(butylene succinate) (PBS), poly(butylene adipate) (PBA), ureidopyrimidinone (Upy), poly(sebacoyl diglyceride) (PSeD-U), and/or Polybuthanedithiol 1,3,5-triallyl-1,3,5-triazine-2,4,6(1H,3H,5H)-trione pentenoic anhydride (PBTPA).

In one embodiment, the power harvester comprises at least one receiver (Rx) coil.

In one embodiment, the cardiac module comprises at least one transmission (Tx) coil.

In one embodiment, the Rx coil of the power harvester is wirelessly coupled to the Tx coil of the cardiac module via magnetic induction for receiving power delivery from the cardiac module.

In one embodiment, the stimulation electrode of the bioresorbable module is operably attached to the epicardial interface of the subject's heart, and the Rx coil of the power harvester is operably placed subcutaneously and in vicinity to the cardiac module.

In one embodiment, the Rx coil at least partially overlaps with the Tx coil and is placed within 25 mm of the Tx coil.

In one embodiment, the Rx coil operably receives the power delivery from a tethered wireless charger when the cardiac module is removed from the subject.

In one embodiment, the cardiac module comprises:
a transmission (Tx) coil configured to deliver power to the bioresorbable module; and
a wireless charging coil configured to receive power delivery from an external power source.

In one embodiment, the cardiac module further comprises a Bluetooth low energy (BLE) system-on-chip (SoC), an ECG analog front end (AFE), and/or an RF power amplifier.

In one embodiment, the stimulation electrode comprises an electrode that is dissolvable.

In one embodiment, the electrode operates for more than 30 days before being dissolved.

In one embodiment, the electrode, the Rx coil and the interconnects are formed of a bioresorbable conductor including molybdenum (Mo), zinc (Zn), iron (Fe), tungsten (W), magnesium (Mg), and/or AZ31B (3 wt % Al and 1 wt % Zn) Mg alloy.

In one embodiment, the stimulation electrode further comprises a bioresorbable steroid eluting patch.

In one embodiment, the bioresorbable steroid eluting patch is configured to operably reduce fibrotic tissue growth at an interface between the bioresorbable module and heart tissue.

In one embodiment, the cardiac module operably receives pacing information from the control module regarding the cardiac pacing of the subject's heart.

In one embodiment, the cardiac module operably delivers the pacing information to the bioresorbable module so as to control the cardiac pacing.

In one embodiment, the bioresorbable module operably provides a charge-balanced biphasic waveform.

In one embodiment, the bioresorbable module is stretchable, twistable, and bendable.

In one embodiment, the skin-interfaced module is stretchable, pristinable, and bendable.

In one embodiment, the skin-interfaced module is peelable from the skin of the subject.

In one embodiment, the control module comprises a hand-held terminal.

In one embodiment, the control module has an interactive interface for receiving and displaying information.

In one embodiment, the system operably provides the cardiac pacing for treatment of bradycardia.

In one embodiment, the system operably detects a heart rate of the subject and determines a heart condition based on the heart rate, and initiates the cardiac pacing without the subject's intervention.

In one embodiment, the system is MRI safe.

In another aspect of the invention, the transient closed-loop system for cardiac pacing and/or defibrillator therapy for a subject, comprises a bioresorbable module for cardiac pacing and/or defibrillator therapy; and a network of skin-integrated modules coupled with the bioresorbable module to control cardiac rhythms, track cardiopulmonary status, provide multi-haptic feedback, and enable transient operation with minimal patient burden.

In one embodiment, the bioresorbable module is configured to wirelessly receive power by inductive coupling to pace the subject's heart through an epicardial interface of the heart for epicardial pacing.

In one embodiment, the bioresorbable module comprises a bioresorbable, stretchable epicardial pacemaker operably attached to the epicardial interface; a bioresorbable steroid-eluting patch coupled to the pacemaker and configured to minimize local inflammation and fibrosis; and a bioresorbable power harvester coupled to the pacemaker to power the pacemaker.

In one embodiment, the pacemaker comprises at least one stimulation electrode connected to the power harvester via stretchable interconnects and configured to operably deliver stimuli to the epicardial interface of the subject's heart for the cardiac pacing.

In one embodiment, the power harvester comprises an antenna for delivering power to the pacemaker, wherein the antenna comprises a loop antenna having at least one coil.

In one embodiment, the power harvester further comprises at least one PIN diode electrically coupled between the antenna and the pacemaker.

In one embodiment, the stimulation electrode, the interconnects and the antenna are formed of a bioresorbable conductor including molybdenum (Mo), zinc (Zn), iron (Fe), tungsten (W), magnesium (Mg), and/or AZ31B (3 wt % Al and 1 wt % Zn) Mg alloy.

In one embodiment, the bioresorbable module further comprises top and bottom encapsulating layers formed of a bioresorbable dynamic covalent polyurethane (b-DCPU) that define a mechanically stretchable structure sealed by thermally activated dynamic bond exchange reactions. In another embodiment, the top and bottom encapsulating layers are formed of polylactic acid (PLA), polyglycolic acid (PGA), polyglycolide (PGL), polycaprolactone (PCL), poly(glycerol sebacate) (PGS), poly(octamethylenemaleate (anhydride) citrate)) (POMaC), poly(1,8-octanediol-co-citric acid) (POC), poly(butylene succinate) (PBS), poly(butylene adipate) (PBA), ureidopyrimidinone (Upy), poly(sebacoyl diglyceride) (PSeD-U), and/or Polybuthanedithiol 1,3,5-triallyl-1,3,5-triazine-2,4,6(1H,3H,5H)-trione pentenoic anhydride (PBTPA).

In one embodiment, the bioresorbable module is a fully implantable, bioresorbable module.

In one embodiment, the bioresorbable module dissolves in the subject's body after a period of time.

In one embodiment, the network of skin-integrated modules comprises a set of flexible, skin-interfaced sensors placed on various locations of the body and configured to capture physiological monitoring of the subject, wherein the physiological information comprises electrocardiograms (ECGs), heart rate (HR), respiratory information, physical activity, and/or cerebral hemodynamics; a radiofrequency (RF) module configured to wirelessly transfer the power from an external power source to the power harvester; and a flexible, skin-interfaced haptic actuator configured to communicate via mechanical vibrations.

In one embodiment, the set of flexible, skin-interfaced sensors comprises at least one respiration module, and/or at least one hemodynamic module.

In one embodiment, the radiofrequency (RF) module comprises a cardiac module comprising a wireless charging unit configured to receive the power charged from an external power source; and a transmission (Tx) coil configured to wirelessly transmit the power to the power harvester.

In one embodiment, the system further comprises a control module in wireless communication with the network of skin-interfaced modules for receiving information from the skin-interfaced modules and controlling the skin-interfaced modules.

In one embodiment, the control module comprises a portable device with a software application for real-time visualization, storage, and analysis of data for automated adaptive control.

T In one embodiment, the skin-interfaced haptic actuator comprises a haptic module configured to wirelessly receive tactile information, and provide at least one pattern of vibro-tactile according to the tactile information.

In one embodiment, the control module operably calculates the at least one regular heart rate according to the physiological information collected by the respiration module and the hemodynamic module, and provides autonomous and wireless pacing therapy to the subject according to the at least one regular heart rate.

In one embodiment, the at least one regular heart rate comprises a high rate limit and a low rate limit.

In one embodiment, when a heart rate of the subject is lower than the low rate limit, the control module activates the bioresorbable module to provide electrical stimulation to the heart at a pre-specified rate, for autonomous and wireless pacing therapy.

In one embodiment, when the heart rate of the subject is higher than the high rate limit, the bioresorbable module remains inactive.

In one embodiment, the control module processes real-time HR and respiratory rate locally and performs cross-checking validation with the transmitted HR and respiratory rate values from networked collection of the skin-interfaced modules.

In one aspect, the invention relates to a method for installing a transient closed-loop system for cardiac pacing and/or defibrillator therapy for a subject. The method comprises coupling at least a part of a bioresorable module to an epicardial interface of the subject's heart for the cardiac pacing; and attaching at least one skin-interfaced module to an outer surface of the subject's skin, wherein the bioresorbable module is in wireless communication with the at least one skin-interfaced module; and a control module is in wireless communication with the at least one skin-interfaced module.

In one embodiment, the at least one skin-interfaced module comprising a cardiac module.

In one embodiment, the cardiac module is placed over skin of the subject's chest area.

In one embodiment, the at least one skin-interfaced module further comprises a hemodynamic module in wireless communication with the control module; and wherein the method further comprises attaching the hemodynamic module to the skin outer surface of the subject.

In one embodiment, the at least one skin-interfaced module further comprises a respiration module in wireless communication with the control module; and wherein the method further comprises attaching the respiration module to the skin outer surface of the subject.

In one embodiment, the at least one skin-interfaced module further comprises a haptic module in wireless communication with the control module; and wherein the method further comprises attaching the haptic module to the skin outer surface of the subject.

In one embodiment, the skin-interfaced module is peelable from the skin of the subject.

In one embodiment, the bioresorbable module dissolves after a period of time.

In one embodiment, the period of time is at least 10 days, 20 days, 30 days, or customizable.

In one embodiment, the bioresorbable module is encapsulated by bioresorbable dynamic covalent polyurethane (b-DCPU), polylactic acid (PLA), polyglycolic acid (PGA), polyglycolide (PGL), polycaprolactone (PCL), poly(glycerol sebacate) (PGS), poly(octamethylenemaleate (anhydride) citrate)) (POMaC), poly(1,8-octanediol-co-citric acid) (POC), poly(butylene succinate) (PBS), poly(butylene adipate) (PBA), ureidopyrimidinone (Upy), poly(sebacoyl diglyceride) (PSeD-U), and/or Polybuthanedithiol 1,3,5-triallyl-1,3,5-triazine-2,4,6(1H,3H,5H)-trione pentenoic anhydride (PBTPA).

In one embodiment, the bioresorbable module comprises a power harvester configured to operably receive power delivery from the cardiac module; a stimulation electrode configured to operably deliver stimulus to the epicardial interface of the subject's heart for the cardiac pacing; and a stretchable interconnect connecting the power harvester and the stimulation electrode.

In one embodiment, the power harvester comprises at least one receiver (Rx) coil.

In one embodiment, the cardiac module comprises:
a transmission (Tx) coil for delivering power to the bioresorbable module; and a wireless charging coil configured to receive power delivery from an external power source.

In one embodiment, the method further comprises wirelessly coupling the Rx coil of the power harvester to the Tx coil of the cardiac module such that the bioresorbable module receives power delivery from the Tx coil of the cardiac module via magnetic induction.

In one embodiment, the stretchable electrode of the bioresorbable module is attached to a surface area of the subject, and the Rx coil of the power harvester is placed subcutaneously and in vicinity to the cardiac module.

In one embodiment, the Rx coil of the power harvester receives power delivery from a tethered wireless charger when the cardiac module is removed from the subject.

In one embodiment, the stimulation electrode comprises an electrode that is dissolvable.

In one embodiment, the electrode is formed of a bioresorbable conductor including molybdenum (Mo), zinc (Zn), iron (Fe), tungsten (W), magnesium (Mg), and/or AZ31B (3 wt % Al and 1 wt % Zn) Mg alloy.

In one embodiment, the stimulation electrode further comprising a bioresorbable steroid eluting patch.

In one embodiment, the bioresorbable steroid eluting patch is configured to operably reduce the fibrotic tissue growth at the interface between the bioresorbable module and epicardial interface.

In one embodiment, the control module comprises a hand-held terminal.

In one embodiment, the control module has an interactive interface for receiving and displaying information.

In another aspect, the invention relates to a method of cardiac pacing and/or defibrillator therapy for a subject with a transient closed-loop system having a bioresorable module, at least one skin-interfaced module and a control module. The method comprises wirelessly transmitting at least one parameter of cardiac pacing from the control module to the at least one skin-interfaced module; wirelessly transmitting the at least one parameter from the at least one skin-interfaced module to the bioresobable module; and pacing the subject's heart by the bioresorbable module according to the at least one parameter.

In one embodiment, the at least one skin-interfaced module comprising a cardiac module placed over skin of the subject's chest area.

In one embodiment, the at least one skin-interfaced module further comprising a respiration module in wireless communication with the control module.

In one embodiment, the at least one skin-interfaced module further comprising a hemodynamic module in wireless communication with the control module.

In one embodiment, the at least one skin-interfaced module further comprising a haptic module in wireless communication with the control module.

In one embodiment, the method further comprises transmitting haptic information to the haptic module by the control module.

In one embodiment, the haptic information comprises at least one pattern of vibro-tactile.

In one embodiment, the haptic module vibrates according to the pattern of vibro-tactile received.

In one embodiment, the wireless communication between the control module and the cardiac module is via a Bluetooth low energy (BLE) protocol.

In one embodiment, the wireless communication between the cardiac module and the bioresorbable module is via at least one of a Bluetooth low energy (BLE) protocol and a near field communication (NFC) protocol.

In one embodiment, the method further comprises collecting hemodynamic physiological information of the subject by the hemodynamic module; and wirelessly transmitting the hemodynamic physiological information to the control module.

In one embodiment, the method further comprises collecting respiration physiological information of the subject by the respiration module; and wirelessly transmitting the respiration physiological information to the control module.

In one embodiment, the method further comprises calculating at least one regular heart rate by the control module based on the physiological information received; adjusting the at least one parameter for cardiac pacing by the control unit based on the physiological information; and providing the at least one parameter to the cardiac module by the control unit.

In one embodiment, the method further comprises wirelessly transmitting the at least one parameter from the cardiac module to the bioresobable module; and pacing the subject's heart by the bioresorbable module according to the at least one parameter.

In one embodiment, the at least one regular heart rate comprising a high rate limit and a low rate limit.

In one embodiment, when a heart rate of the subject detected by the cardiac module is lower than the low rate limits, the system activates the bioresorbable module, to provide electrical stimulation to the heart at a pre-specified rate.

In one embodiment, when the heart rate of the subject detected by the system is higher than the high rate limits, the bioresorbable module remains inactive.

In one embodiment, the control module calculates at least one regular heart rate according to the respiration and hemodynamic information collected by the respiration module and the hemodynamic module and provides autonomous cardiac pacing to the subject according to the at least one regular heart rate.

In one embodiment, the method further comprises initiating the cardiac pacing without the subject's intervention.

In one embodiment, the control module processes real-time HR and respiratory rate locally and performs cross-checking validation with the transmitted HR and respiratory rate values from networked collection of skin-interfaced modules including the cardiac, respiratory, and hemodynamic modules.

These and other aspects of the invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

The accompanying drawings illustrate one or more embodiments of the invention and together with the written description, serve to explain the principles of the invention. Wherever possible, the same reference numbers are used throughout the drawings to refer to the same or like elements of an embodiment.

FIG. 1 illustrates a transient closed-loop system for temporary cardiac pacing according to embodiments of the invention. Panel A: Schematic illustration of the system for (i) autonomous and wireless pacing therapy and (ii) nonhospitalized termination. Panel B: Operational diagram of the closed-loop system for continuous monitoring, autonomous treatment, and haptic feedback. Panel C: Photographs showing the sizes of the various modules, relative to a U.S. quarter. Panel D: Photographs of a bioresorbable module at different time points during immersion in a simulated biofluid (in PBS at 95° C.).

FIG. 2 illustrates materials, design features of the key components of the transient closed-loop system for cardiac electrotherapy according to embodiments of the invention. Panel A: Schematic illustration of a bioresorbable module. Panel B: S₁₁ values of the Rx coils with different diameters (d_{coil}). Panel C: Example output waveform (red; d_{coil}=12 mm) wirelessly generated by an alternating current (black; about 6 Vpp; 13.56 MHz) applied to the Tx coil. Panel D: Output open circuit voltage (VOC) of devices as a function of tensile strain (left) and twist angle (right) at a fixed transmitting voltage (8 Vpp) and frequency (13.56 MHz). Panel E: Drug-release behaviors of steroid-eluting patches with three different ratios of base polymer. Error bars represent standard deviation. Panel F: Measurements of VOC of the bioresorbable module (red squares; 10-mm-thick Mo) and a reference module (black circles; 700-nm-thick W coated 50-pm-thick Mg) immersed in PBS (37° C.). Panel G: Schematic illustration of a skin-interfaced cardiac module. PMIC, power management integrated circuit. Panel H: System block diagram of the cardiac module. Panels I-L: Comparisons of ECG, HR, respiratory rate, and SpO₂ levels determined by the skin-interfaced modules (red; cardiac module in panels I-J; respiratory module in panel K; hemodynamic module in panel L) and a reference device (black). In panel L, data were collected from a healthy subject who held their breath for 60 s (yellow background). A.U., arbitrary units; rpm, respirations per minute.

FIG. 3 illustrates a process of treatment for temporary bradycardia using the transient closed-loop system in an ex vivo Langendorff-perfused human whole heart model according to embodiments of the invention. Panels A-B: Schematic illustration (panel A) and photograph (panel B) of a Langendorff-perfused human whole-heart model with a transient closed-loop system (d_{coil}=25 mm). Panel C: Action potential maps obtained by optical mapping of the human epicardium. Panel D: Flow chart of closed-loop hysteresis pacing to activate the pacemaker upon automatic detection of temporary bradycardia. Panel E: Programmed HR (top) and measured ECG (bottom) of a human whole heart. Set parameters are as follows: The lower rate limit is 54 bpm, pacing duration is 10 s, and pacing rate is 100 bpm.

FIG. 4 illustrates patient feedback and adaptive pacing functions of the transient closed-loop system for cardiac electrotherapy according to embodiments of the invention. Panel A: Schematic illustration of a transient closed-loop system with the full collection of skin-interfaced modules. Panel B: Demonstration of the patient-awareness function using a multihaptic module. Accelerometer data (g) corresponds to vibrations (z axis) of the haptic actuators. Panel C: Results of clinical tests with a healthy human subject: (i) calculated physical activity and (ii) respiratory rate using data from the respiratory module, (iii) comparison of the HR (black) of a healthy human subject monitored by the cardiac module and rate-adaptive pacing signals (red) processed from the algorithm, (iv) calibrated and measured changes in core body temperatures using data from the respiratory module, and (v) representative SpO₂ measurements from the hemodynamic module.

FIG. 5 illustrates envisioned use case scenarios of the transient closed-loop system according to embodiments of the invention. Scenario 1: A patient presents symptoms indicating need for electronic pacemaker implantation. Scenario 2: A bioresorbable module is implanted with electrodes attached to the heart muscle. The receiver (Rx) coil of the device is placed in a subcutaneous pocket. Scenario 3: Throughout the treatment period, the skin-interfaced cardiac and respiratory modules are placed on the chest and suprasternal notch, respectively. The other skin-interfaced devices, such as hemodynamic and haptic modules, can also be included. The patient's mobility and daily activities are only minimally impeded due to the wireless nature of the system. Scenario 4: These and other features of the system enable the patient to complete inpatient treatment quickly for early hospital discharge. Scenario 5: The transient closed-loop system provides continuous monitoring and autonomous treatment through coordinated operation of (i) an implanted bioresorbable module, (ii) a collection of skin-interfaced modules, and (iii) an external control module. For example, if the external control module detects bradycardia from the real-time ECG data, the skin-interfaced cardiac module delivers power and control signals to the implanted bioresorbable module via inductive wireless energy transfer to pace the heart. Scenario 6: Two sets of each skin-interfaced module and one tethered stimulator can be provided to allow continuous electrical stimulation. The battery in the skin-interfaced modules can be recharged wirelessly. Scenario 7: Wireless and automatic transfer of diagnostic health information to a clinician enables remote medical care. This platform may allow clinicians to detect changes early and to make proactive decisions, to improve patient outcomes. Scenario 8: Following resolution of pacing needs or insertion of a permanent device, the implanted device dissolves into the body, thereby eliminating the need for surgical extraction. Scenario 9: The skin-interfaced modules can be physically removed by gentle peeling from the skin, terminating the medical treatment without the need to return to the hospital.

FIG. 6 illustrates in vivo bioresorbability of the device with different constituent materials according to embodiments of the invention. Three-dimensional computed tomography (CT) images of rats collected over 9 weeks after implantation of a bioresorbable device (i.e., reference module) with W/Mg bilayer electrodes (W, about 700 nm thick; Mg, about 50 μm thick). The images show the gradual disappearance of the devices to the stage where they are no longer visible on day 62. Scale bar, 10 mm. n=3 biologically independent animals.

FIG. 7 illustrates the design of the bioresorbable module according to embodiments of the invention. Panel A: Three devices with Rx coils with different diameters: (top) 25 mm (5 turns), (middle) 18 mm (7 turns), (bottom) 12 mm (12 turns). Panels B-C: Dimensions of the device with coil diameter of (panel B) 25 mm and (panel C) 12 mm: (top) x,y-view; (bottom) x,z-view, (right) magnified image of the contact pad. The total length can be altered to meet requirements for the target application, simply by changing the length of the extension electrode.

FIG. 8 illustrates electromagnetic performance characteristics of the wireless power transfer system according to embodiments of the invention. Electromagnetic characteristics of bioresorbable modules with Rx coils with three different diameters (black, 12 mm; red, 18 mm; orange, 25 mm). The modules are placed in the air without any connection to the load resistance. Panels A-B: (panel A) Simulated and (panel B) experimentally achieved and frequency dependent S₁₁ parameter for the wireless systems. Panel C: Simulated Q factors for the wireless power harvesting units. Panel D: The frequency-dependent inductance (L) of the wireless stimulator.

FIG. 9 illustrates electrical characteristics of the bioresorbable module according to embodiments of the invention. Panel A: Output voltage of the bioresorbable module with three different Rx coil diameters (black, 12 mm; red, 18 mm; orange, 25 mm) with a load resistance of 1 kΩ as a function of the Tx coil (3 Turns, 30 mm diameter) driving frequency. Input voltage, 5 Vpp. Panel B: Output voltage (input frequency=13.5 MHz; load resistance=1 kΩ) of each Rx coil as a function of input voltage that drives the Tx coil (3 Turns, 30 mm diameter) using a waveform generator.

FIG. 10 illustrates experimental and theoretical aspects of MRI compatibility of the device according to embodiments of the invention. Panel A: Experimental setup for the MRI compatibility test. The bioresorbable module is placed on the chicken thigh subcutaneously. LED is connected on the exposed electrode of the module and serves as an indicator for the electrical stimulation (e.g., heart pacing). Panel B: MR image (y-z cross-section) from the chicken thigh with the bioresorbable module. i, ii indicate the cross-sectional direction for the additional MRI scan. Scale bar, 10 mm. Panel C: MR images from the x-z cross-section of Rx coil (i) and extension electrode (ii). Dotted line indicates the area of MR image distortion (i.e., shadow). Scale bar, 10 mm. Panel D: Calculated in-plane (left) and out-of-plane (right) gradients of the magnetic field induced by the bioresorbable pacemaker at 128 MHz.

FIG. 11 illustrates computational results for the maximum change in temperature of the bioresorbable module during an MRI scan according to embodiments of the invention. Panel A: Maximum temperature change of the Mo layer of the device (red, bioresorbable module) and heart tissue (black) versus time for an MRI scan. Panel B: Distribution of the temperature change on the heart tissue interface with the bioresorbable module at 0.17 s which corresponds to the time of the maximum temperature change on the heart tissue.

FIG. 12 illustrates mechanical reliability of the bioresorbable module according to embodiments of the invention. Finite element analysis (FEA) reveals the distributions of principal strain for compression-induced buckling perpendicular to the lengths of the interconnects. (Left) Photographs of a bioresorbable module with b-DCPU-encapsulated serpentine electrodes during uniaxial stretching, twisting and bending. Scale bar indicates 10 mm. (Right) Corresponding three-dimensional FEA results. FEA reveals that the maximum strains in the Mo electrodes and b-DCPU encapsulation are less than 0.6% for stretching (26%), twisting (612°), and bending (60%).

FIG. 13 illustrates changes in the performance of the device as a function of coil-to-coil distance according to embodiments of the invention. Electromagnetic characteristics of bioresorbable modules with Rx coils with three different diameters (black, 12 mm; red, 18 mm; orange, 25 mm). The modules are placed in the air without any connection to the load resistance. Panel A: Simulated power transfer efficiency as a function of changes in coil-to-coil distance. Panel B: Experimental results for the open circuit voltage (Voc) as a function of the distance between the Tx and Rx coils (input voltage, 4.0 Vpp; input frequency, resonant frequency of each coil). Panel C: Experimental results for the open circuit voltage (VOC) as a function of the center-to-center horizontal displacement between the Tx coil (blue; 12 mm diameter; 4 turns) and Rx coil (red; 12 mm diameter). Input voltage, 10 Vpp.

FIG. 14 illustrates the bioresorbable steroid eluting patch of the bioresorbable module according to embodiments of the invention. Changes in released mass of DMA as a function of time from composites with different polymer matrixes: Panel A: PLGA 75:25; Panel B: PLGA 65:35; and Panel C: PLGA 50:50. Different colors indicate different patch samples. As expected, release profiles of the steroid eluting devices in solution correlate with the rate of polymer degradation, where the lactide:glycolide ratio of 50:50 offers the fastest kinetics followed by the 65:35 and then 75:25 formulations. Due to spatial variations in the rate of evaporation on the wafer (i.e., coffee ring effect), the concentration of the drug in each patch varies depending on their position across the wafer. As a result, the samples, especially those based on PLGA 50:50, show saturation points that are higher or lower than the calculated value of 100% drug released estimated from the area of the patch (dimensions: 4.64×3.34 mm2). In panel C, the decline in the value of mass released after day 30 is attributed to the degradation of the 50:50 PLGA matrix and resulting effects on the UV-vis absorbance. The release rate and saturation point (i.e., maximum amount of drug release) can be tuned to the time period of clinical need by changing the type of PLGA (i.e., ratio between lactic and glycolic acid) and the amount of drug.

FIG. 15 illustrates changes in output voltages during biodegradation of the bioresorbable module according to embodiments of the invention. Measurements of output voltages of bioresorbable module with 10 μm thick Mo electrodes in PBS at 37° C. (red square) and 40° C. (orange square) and with W/Mg electrodes (700 nm/50 μm thickness) in PBS at 37° C. (black circle) and 40° C. (grey circle). Devices at two different temperatures across this range show similar functional lifetimes since the rates of degradation of the metal electrodes and the water permeability of b-DCPU do not change significantly over this relatively small temperature range (ΔT=3° C.).

FIG. 16 illustrates time sequence of images that show the process of degradation of the bioresorbable module according to embodiments of the invention. Photographs at various stages of the dissolution of a bioresorbable module in PBS (pH 7.4) at 37° C. (left) Reference module with 700 nm thick W coated 50 μm thick Mg electrode and (right) bioresorbable module with 10 μm thick Mo electrodes. Scale bar, 10 mm.

FIG. 17 is a schematic illustrations of fPCB layout for the cardiac module according to embodiments of the invention. Illustration of the fPCB that integrates a wireless charging coil (Rx), central body (a power management system, a Bluetooth low energy SoC, an ECG analog front end (AFE), an RF power amplifier, and a lithium-polymer battery), and a wireless coil to power the bioresorbable module (Tx). Red and blue arrows indicate folding orientation: folding up (blue), folding down (red). RA, LA, and RL denote right arm, left arm, and right leg electrodes, respectively.

FIG. 18 illustrates mechanical flexibility of the cardiac module according to embodiments of the invention. Panel A: FEA computations for the fPCB. Panel B: Images and FEA results during various mechanical deformations: undeformed, stretching, and bending (convex and concave). Mechanical flexibility of the skin-interfaced module. Images (Left), FEA of the encapsulated device (middle), and fPCB (right). On the basis of the layouts and the mechanical moduli, the maximum strains in the serpentine-shaped traces of the fPCB are less than 0.3% for a stretching (17%) and bending (radius=3.1 mm).

FIG. 19 illustrates normalized signal differences for various placements of the cardiac module according to embodiments of the invention. Panel A: Schematic illustration of the cardiac module: (left) front and (right) back sides. Panel B: Illustration of the cardiac module placement. Panel C: Representative ECG signal morphology from four different placements and device angles: (i, black) near shoulder with no rotation, (ii, red) on chest with no rotation, (iii, orange) on chest with 90° rotation, (iv, green) on chest with 180° rotation. In practical use, a chest band can be placed over the cardiac module to prevent accidental detachment. Of the three electrodes (RA, LA, RL) for ECG recordings, one that acts as a right leg drive amplifier to generate an inverted current of common-mode signal for the other two which serve as inputs to an instrumentation amplifier. This three-electrode configuration counteracts common-mode voltage variations, such as noise from body motion, which thus enhances the signal-to-noise ratio by increasing the common-mode rejection ratio (CMRR). As a result, clean ECG waveforms can be obtained despite vigorous body motions by passing through an active 2-pole high-pass filter (fcut-high=0.5 Hz) and a 3-pole low-pass filter (fcut-low=50 Hz).

FIG. 20 is a block diagram of signal processing for extracting real-time heart rate using in-sensor Pan-Tompkins algorithm of the cardiac module according to embodiments of the invention.

FIG. 21 is a block diagram of signal processing for wireless (left) ECG monitoring, (right) body motion and temperature monitoring on the cardiac and respiratory modules, respectively, according to embodiments of the invention.

FIG. 22 is a Bland-Altman plot for (panel A) heart rate and (panel B) respiratory rate collected from three healthy adults using the transient closed-loop system and a clinical-standard system according to embodiments of the invention. Bland-Altman plot for (panel A) heart rate (HR; mean difference=0.66 beats per minute (bpm); SD=1.30 bpm) and (panel B) respiratory rate (RR; mean difference=0.27 respiration per minute (rpm); SD=0.99 rpm) collected from three healthy adults using a transient closed-loop system and a clinical-standard system. (A) 2541 data points (panel B) 1885 data points.

FIG. 23 illustrates AES-128 encryption/decryption architecture of the device according to embodiments of the invention. BLE 4.2 LE secure connection allows sensor communication to be secured with the 128-bit Advanced Encryption Standard (AES-128) authenticate-and-encrypt block cipher mode (CCM), protecting against attacking and spoofing for patient privacy and device safety. The plain text (input) data is encrypted by substituting nonlinearly byte-by-byte (SubBytes), shifting the last three rows (ShiftRows), producing new columns with mixed data (MixColumns), and adding round key (AddRoundKey) to generate cipher text. The encrypted cipher text data is decrypted with inverse ShiftRows (InvShiftRows), inverse SubBytes (InvSubBytes), AddRoundKey, and inverse MixColumns (InvMixColumns) steps.

FIG. 24 is an illustrations of waveforms for RF signals transmitted via the powering coil according to embodiments of the invention. Panel A: Block diagram of RF power generator on the wireless skin-interfaced module. Panel B: The control signal for cardiac pacing passes from the microcontroller unit (MCU) in Bluetooth System-on-Chip (SoC) via GPIO to an on/off enable crystal oscillator (13.56 MHz). The GPIO signal defines an accurate pacing period (TPM) and pacing duty cycle (TON). Following the inverter, a controlled gate voltage of the enhancement-mode power MOSFET drives an output power. Panel C: Drive signal generated via the Tx coil, placed on the wireless skin-interfaced module. Panel D: Rectified signal on the bioresorbable module. The user-interface allows automatic adjustments to TPM and TON depending on wearer's biometrics.

FIG. 25 illustrates heart pacing with the closed-loop system in an in vivo canine model according to embodiments of the invention. Panel A: In vivo studies with a canine whole-heart model at a scale that recapitulates human physiology validate the envisioned clinical implementation since the canine cardiovascular system bears a high resemblance to that of humans. A 6-lead ECG system with adhesive-backed electrodes on the limbs monitors cardiac activity throughout the period of the experiments to serve as a reference. The contact electrode of the bioresorbable module interfaces with the myocardium, the extension electrode threads through the intercostal space and the power harvesting receiver lies within a subcutaneous pocket on the ventral surface of the dog. The Tx coil in the skin-interfaced module mounts on the skin at a positioned aligned with the Rx coil of the bioresorbable module. Panels B-C: Photographs of an open-chest procedure with the contact electrode of the bioresorbable module sutured to the ventricular epicardium (panel B) and with placement of the skin-interfaced cardiac module next to the sutured incision after chest closure (panel C). Scale bars, 20 mm. Panel D: Input pulse waveform for cardiac pacing generated by a skin-interfaced cardiac module. TPM, 333 ms; TON, 6.6 ms; sinusoidal waveform, 13.56 MHz; input voltage, 6 Vpp. (E) Corresponding monophasic output waveform (about 4.5 mW; pulse frequency, 3 Hz; pulse width, 6.6 ms) wirelessly generated by the bioresorbable module (25 mm Rx diameter). (F) 6-lead ECG recording of intrinsic sinus rhythm (white background; about 150 bpm) and ventricularly paced rhythm (yellow background; about 180 bpm) defined by the system. Operation of the system induces a transition of ECG signals from narrow intrinsic QRS complexes to widened, amplified QRS complexes with shortened R-R intervals. This pattern indicates a transition from normal sinus rhythm excitation via the cardiac conduction system to a ventricularly paced excitation not involving the conduction system.

FIG. 26 illustrates an inductive coupling between the bioresorbable module and cardiac module according to embodiments of the invention. Panel A: Schematic illustration of the conditions for the electromagnetic simulation. The Rx coil (12 mm diameter) of the bioresorbable module is placed on the Tx coil of the cardiac module. Panel B: The frequency-dependent inductance (L) of the (red) Rx coil of the bioresorbable module and (blue) Tx coil of the cardiac module. Panel C: Simulated S11 parameter of the wireless system with a load resistance of 50 kΩ in (blue) air and on the (red) heart. Panel D: Simulated S21 parameter of the wireless system with a load resistance of 50 kΩ in (blue) air and on the (red) heart. Panel E: Dimensions for electromagnetic simulation for a mesh model of a human body. Simulated electric field at an input power (Tx coil) of 1 W as a function of position across a mesh model of a human body. A distance of 2.5 cm corresponding to the distance between Tx and Rx coils through the skin and underlying tissue is assumed. (left) 3D and (right) 2D (y,z-axis) view.

FIG. 27 illustrates electromagnetic characteristics of the transient closed-loop system in an in vivo human heart model according to embodiments of the invention. Panel A: Dimensions for electromagnetic simulation. The Rx coil (25 mm diameter) of the bioresorbable module is placed in a subcutaneous pocket, and the Tx coil of the skin-interfaced module is positioned on the skin aligned to the Rx coil. A 2.5 cm distance between Tx and Rx coils is assumed. Panel B: Simulated specific absorption rate (SAR; a measure of the rate at which RF energy is absorbed by the body) at an input power (Tx coil) of 1 W as a function of position across a mesh model of a human body. (left) 3D and (right) 2D (x,y-axis) view. For both the specific absorbed radiation (SAR) and the maximum permissible exposure, values are lower than regulation limits by roughly a factor of 10.

FIG. 28 is a schematic illustration of a continuous pacing setup according to embodiments of the invention. The position of the cardiac module is fixed using a home-made vest.

FIG. 29 illustrates a process of preparation for the in vivo non-anesthetized continuous pacing test in a rat model according to embodiments of the invention. Panel A: Surgical procedure for implanting a bioresorbable module in a small animal (rat) model. Here, the bioresorbable module (12 mm coil diameter) inserts through an incision in the intercostal space to access the thoracic cavity and interface to the heart. The Rx part of the module remains in the subcutaneous space. The electrode pads laminate onto the anterior epicardium of the left ventricle and are secured by suture. Panel B: (i) The location of the subcutaneous Rx coil of the implanted bioresorbable module is identified as the location on which the skin-interfaced module should be placed. Gold-standard ECG are connected in a Lead I configuration (positive electrode on right, negative electrode on left, ground electrode on lower limbs) as a reference signal. (ii) The area on which the skin-interfaced module attaches is shaved to expose the skin. The skin-interfaced module is placed in direct contact with the skin. (iii) A custom vest ensures contact between the skin-interfaced module and the skin for recording high quality ECG signals. The vest also maintains the location of the device so that power can be effectively transferred to the bioresorbable module. (iv) A mobile application (external control module) is wirelessly connected by Bluetooth to the skin-interfaced module. The application shows ECG data collected in real-time for monitoring of the heart rhythm. The user selects the pacing settings from this interface.

FIG. 30 illustrates current consumption of the cardiac module while Bluetooth advertising, Bluetooth data streaming, and continuous recording according to embodiments of the invention. Panel A: Average and maximum levels of current for 40 s: (0-10 s) Real-time data streaming with continuous ECG (500 Hz) along with pacemaking signal (TPM=1000 ms; TON=5 ms), (10-20 s) ECG (500 Hz) along with pacemaking signal (TPM=500 ms; TON=5 ms), (20-30 s) ECG (500 Hz) without pacemaking signal, and (30-40 s) Bluetooth (BT) advertising. Although the pacing signal demands high current (33-39 mA), duration of a pacing pulse (TON) is only 1-7 ms (adjustable), which leads to negligible total power consumption compared to that associated with BT transmission. The device connects to the user interface (tablet). Panel B: Magnified views of the four different conditions.

FIG. 31 illustrates battery life-time measurement result according to embodiments of the invention. Panel A: Segmented ECG waveforms from data collected continuously over 74 hours with a regular-sized 80 mAh battery. Panel B: Magnified ECG waveform for three different days. Panel C: Recorded continuous temperature data for 72 hours. Measured thermocouple data on the cardiac module (black) and the surrounding environment (red). The temperature of the cardiac module fluctuates according to changes in ambient temperature due to the cardiac module's constant internal temperature.

FIG. 32 illustrates a switching scenario for recharging skin-interfaced module according to embodiments of the invention. Panel A: Photograph of a skin-interfaced module and tethered stimulator. Scale bar, 5 cm. Panel B: Simulated specific absorption rate (SAR; input power of skin-interfaced module=1 W; input power of tethered coil=2 W) as a function of position across a mesh model of a human body. Scale bar, 5 cm. Under these conditions, the SAR remains below IEEE and Federal Communications Commission (FCC) guidelines(32). Panel C: Schematic illustration of the module replacement scenario during continuous pacing treatment. (I) Continuous monitoring and cardiac pacing using skin-interfaced module-A. (II) Approaching the tethered stimulator (power on) above the skin-interfaced module, and turning off the module-A. (III) Removing module-A, and placing it on the wireless charging device. (IV) Attaching fully charged module-B. (V) Removing the tethered stimulator. Panel D: ECG recording of intrinsic rhythm (about 150 bpm) and ventricular capture (about 180 bpm) in a canine model during the switching scenario.

FIG. 33 illustrates a switching scenario in an in vivo rat model according to embodiments of the invention. Panel A: ECG signals recorded by the cardiac module during continuous pacing with the implanted bioresorbable pacemaker for 2 days. Panel B: ECG recording of continuous ventricular capture (about 600 bpm) in a rat model during exchange of the cardiac module (switching scenario).

FIG. 34 illustrates temperature measurement during electrical stimulation according to embodiments of the invention. Panel A: Experimental setup for temperature measurement during electrical stimulation for heart pacing. An ECG simulator generates (8) an ECG signal for the skin-interfaced module, then a control module (3) displays the measured ECG signal and sends pacing parameters (frequency=1000 ms; pulse width=5 ms) to the skin-interfaced module. The skin-interfaced module continuously powers the bioresorbable module, and an oscilloscope (5) that is connected to the contact electrodes of the device monitors the resulting output voltage (about 7 V). Two devices (infrared (IR) camera (1) and k-type thermocouple (6)) measure the real-time changes in temperature and display the results on the monitor (4) or indicator, respectively. The power profiler kit (7) measures the power consumption (about 1.14 mA) of the skin-interfaced module and displays the result in the monitor (2). Panels B-C show the IR images of the skin-interfaced module and bioresorbable module with temperature of the Rx part of the bioresorbable module read by thermocouple before and after (180 min.) continuous operation of the system, respectively.

FIG. 35 illustrates temperature measurement while charging a skin-interfaced cardiac module according to embodiments of the invention. Panel A: Experimental setup for temperature measurement with three k-type thermocouples from three different locations before (left) and after (right) charging of the skin-interfaced cardiac module: (T1) on the skin-interfaced cardiac module (i.e., device); (T2) on the charger; (T3) ambient. Panel B: Recorded continuous temperature changes from 0 min to 360 min (until battery full charging). Pictures with temperature readings from three thermocouples (top row) and an IR camera (bottom row). Panel C: Measured thermocouple data on the device (blue), on the charger (red), and the ambient temperature (green).

FIG. 36 illustrates setup and results of simulation for chronic pacing study in an in vivo rat model according to embodiments of the invention. Panel A: The RF system (i.e., power and stimulation controller) connects to a Tx loop coil (2 turns) outfitted on the outer surface of a custom-built plastic cage to deliver power to the implanted bioresorbable module. The animal with an implanted device can move freely, to enable continuous in vivo pacing of the heart. Panel B: (top) Magnetic field distribution (input power=8 W) inside the cage at a cross sectional plane that intersects a simple model of a rat. (left) 3D and (right) 2D (y,z-axis) view. (bottom) Simulated specific absorption rate (SAR) as a function of position across a mesh model of a rat body. (left) 3D and (right) 2D (x,y-axis) view of the rat model. Under these conditions, the SAR remains below IEEE and Federal Communications Commission (FCC) guidelines.

FIG. 37 is daily pacing demonstration in an in vivo rat model according to embodiments of the invention. ECG signals before (white background) and during pacing (yellow background; red arrows indicate delivered electrical stimuli) with the implanted bioresorbable module (left) 17 and (right) 32 days after implantation.

FIG. 38 illustrates changes in wireless pacing behavior during in vivo chronic tests in a rat model according to embodiments of the invention. ECG signals from rats with bioresorbable module (non-stretchable device with steroid system) at 3 and 4 days after device implantation. Yellow background and red triangles indicate the successful pacing period and timing of wireless electrical stimulation, respectively.

FIG. 39 illustrates mason's trichrome staining of tissues near the site of implantation according to embodiments of the invention. Panel A: Representative images of Masson's trichrome stains of the cross-sectional area of the anterior left ventricle near the site of implantation. Pink indicates myocytes, blue indicates fibrotic tissue, and white indicates interstitial space. (left) A control rat without an implanted device, (middle) a rat after 4 weeks of pacing treatment using an implant without a steroid eluting component, and (right) a rat after 4 weeks with a steroid eluting component. Scale bar, 1 mm. Panel B: Quantitative analysis of Masson's trichrome stained cross sections. Percent volume of myocytes (pink), interstitial space (white), and fibrosis (blue) in the transmural cardiac cross-section of naïve rats and rats 4 weeks following pacing treatment with and without the steroid eluting component. A significant increase in fibrotic tissue appears in rats with non-steroid eluting devices. The steroid-eluting component eliminates this increase, as compared to the control group. Kruskal-Wallis test: myocytes: H(2), 5.134, p=0.0768; interstitial space: H(2), 0.4518, p=0.7978; fibrosis: H(2), 7.229, p=0.0269. Dunn's multiple comparison test at a significance level of 0.05, *p=0.0219. n=10, 12, 7 biologically independent animals per group, respectively.

FIG. 40 illustrates inflammatory response of myocardium following implantation of the bioresorbable module according to embodiments of the invention. Panel A: Representative images of the stained cross-sectional area of the left ventricle of a rat with no pacemaker implanted (control), with a non-steroid (DM, dexamethasone) eluting pacemaker, or a steroid (DM) eluting pacemaker. Arrowheads indicate CD45+ cells. Scale bar, 100 μm. Panel B: Frequency of CD45+ cells in the ventricular myocardium near the site of implantation. No significant difference in CD45+ cells in the myocardium following pacemaker implantation. Kruskal-Wallis test, H(2)=0.1871, P=0.9213. Dunn's multiple comparison test at a significance level of 0.05, P>0.9999. n=6 biologically independent animals per group. Data are presented as mean values ±SD.

FIG. 41 illustrates changes in weights of animals after implantation of the bioresorbable modules according to embodiments of the invention. Weights of animals with (experimental group; red circle) and without implanted device (control group; black square) measured every 3-8 days following surgery show an increase appropriately with age, which is as expected in healthy animals over time. n=3 biologically independent animals per group.

FIG. 42 illustrates echocardiographic assessment of animals following implantation of the bioresorbable module according to embodiments of the invention. Panel A: Representative M-mode echocardiograms (top) before and (bottom) 7 weeks after surgery. Panels B-H: No significant changes in ejection fraction, stroke volume, diastolic volume, diastolic diameter, fractional shortening, systolic volume, systolic diameter, or cardiac output when compared before or at 1-, 3-, 5- or 7-weeks following operation. Friedman's test: ejection fraction (χ_{F}²(2)=2.267, P=0.6868), stroke volume (χ_{F}²(2)=6.667, P=0.1546), diastolic volume (χ_{F}²(2)=8.133, P=0.0868), diastolic diameter (χ_{F}²(2)=8.84, P=0.0652), fractional shortening (χ_{F}²(2)=2.267, P=0.6868), systolic volume (χ_{F}²(2)=6.000, P=0.1991), systolic diameter (χ_{F}²(2)=6.000, P=0.1991), cardiac output (χ_{F}²(2)=4.667, P=0.3232). Dunn's multiple comparison test at a significance level of 0.05. Paired data measurements for n=6 biologically independent animals. These measurements show that the bioresorbable module does not impair the mechanical function of the heart. Data are presented as mean values ±SD.

FIG. 43 illustrates BNP 45 assessment of the animals before and after the bioresorbable module according to embodiments of the invention. No significant differences in levels of brain natriuretic peptide 45 (BNP 45) before and 3 weeks after bioresorbable module implantation, which indicates that no substantial level of heart failure is induced. Mann Whitney test, P=0.3429. n=6 biologically independent animals per group.

FIG. 44 illustrates blood chemistry for rats with and without the implanted device according to embodiments of the invention. Analysis of complete blood chemistry for rats with (experimental group) and without implanted device (control group) at 1, 5, 11, 15, and 20 weeks after implantation reveals maintenance of overall healthy physiology in the animals. n=3 biologically independent animals per group. F(3.427, 109.7)=0.7106, P=0.5656. GLU, glucose (P=0.7558, 0.9780, 0.9933, >0.9999); TRIG, triglycerides (P=0.9809, 0.9600, 0.7007, 0.9930); ALT, alanine aminotransferase (P=0.9510, 0.7733, 0.9986, 0.4856); ALP, alkaline phosphatase (P=0.9501, 0.9408, 0.9393, 0.1111); CHOL, cholesterol (P=0.9876, >0.9999, >0.9999, 0.8366); Cl, chloride (P=0.6092, 0.9996, >0.9999, 0.8203); TCO2, bicarbonate (P=0.9977, 0.9903, 0.9997, 0.9914); Na, sodium (P=0.0845, 0.7764, 0.9586, 0.6987); UREA, urea (P=0.9369, 0.6633, 0.9982, >0.9999); PHOS, phosphorus (P=0.7452, 0.5050, >0.9999, 0.9811); Ca, calcium (P=>0.9999, 0.9436, 0.0971, 0.1936); ALB, albumin (P=0.9755, >0.9999, >0.9999, >0.9999); A/G, albumin/globulin; CREA, creatinine (P=0.8577, 0.6092, 0.9369, >0.9999); GLOB, globulin (P=0.6280, 0.6633, 0.3641, 0.9977); K, potassium (P=0.4032, 0.9548, 0.9856, 0.9533); TBIL, total bilirubin; TP, total protein (P=>0.9999, 0.4457, 0.4457, 0.9369).

FIG. 45 illustrates changes in heart rhythm in the ex vivo Langendorff-perfused human whole heart model. External artificial pacing modulates the rhythm of the human whole heart: (top) ECG signals and (bottom) calculated heart rate measured in real time by LabChart software, according to embodiments of the invention.

FIG. 46 is schematic illustrations of (panel A) full scale multi-component system for adult patients and (panel B) minimized components system for young patients. For the neonatal or pediatric patients, reducing the number of modules can be important. For example, the function of both the cardiac module and the respiratory module can be combined into a single unit and the haptic module can be interfaced to a guardian or health provider. The full multi-component system offers, however, the most accurate physiological data (e.g., a respiratory module on the suprasternal notch, a cardiac module on the chest, and hemodynamic module on the forehead) and the greatest degree of redundancy.

FIG. 47 illustrates four different vibration patterns of the multi-haptic module. Schematic illustrations (left), photographs (middle) and acceleration graphs (right) for four different patterns of vibration of the multi-haptic module: (1) entire device; (2) round pattern; (3) cross pattern; (4) arrow pattern. Coordinated control of the four actuators in the module supports various forms of feedback to the patient. The red LED indicates the position of haptic vibration. The duration of the vibration (i.e., vibration time for one event) and the interval for vibration (time between vibrations for one complete set of patterns) can also be adjusted depending on the patterns. For example, the duration and interval of pattern (1) is about 0.2 and 0.5 s, while duration of pattern (2) is around 1.8 s without interval, in a continuous mode.

FIG. 48 illustrates patient awareness function to aid in the process of aligning the skin-interfaced cardiac module to the bioresorbable module. Panel A: Flow chart of the steps implemented in the cardiac module with accelerometer to activate the haptic module. Different patterns of vibrations assist with the process of coil-to-coil alignment and to alert failure of the implanted bioresorbable module to operate. Panel B: Accelerometer data (black, x-axis; red, y-axis; orange, z-axis) corresponding to the vibration of the haptic module during the coil-to-coil alignment scenario. The cardiac module is directly connected to the ECG signal generator (Tech Patient Cardio v4) to mimic a practical situation. The haptic actuator is placed on the respiratory module to measure the vibration during the alignment.

FIG. 49 illustrates rate-adaptive pacing studies of the transient closed-loop systems Schematic illustration of the clinical tests setup for rate-adaptive pacing with healthy human subject. A pneumotachograph, tethered ECG leads, and a pulse oximeter provide reference respiratory rate and ECG as gold standards for comparison. The hemodynamic module monitors the oxygen saturation level. The cardiac and respiratory modules measure ECG and x-, y-, z-axis acceleration respectively. The external control module (tablet computer) calculates the physical activity and respiratory rate and sends rate-adaptive pacing signals determined by the closed-loop algorithm, to the cardiac module. The bioresorbable module placed on the cardiac module generates pacing signals (pulsed output), and an oscilloscope captures these waveforms for comparison to the heart rate of the human subject.

FIG. 50 illustrates a block diagram for rate adaptive pacing. Signal processing for extracting heart rate (HR), heart rate variability (HRV), respiratory rate, and physical activity to generate an adaptive pacing signal on the external control module (tablet computer). The signal power in the 1-10 Hz range serves as a surrogate marker of physical activity. A simple pacing rate model exploits a linear relationship between physical activity, respiratory rate, and the heart rate.

FIG. 51 illustrates results of rate-adaptive pacing studies. Total 8 healthy subjects (male) during cycling for 7 minutes.

FIG. 52 illustrates a block diagram of Proportional-Integral-Derivative (PID) control for self-adaptive output calculation. The difference between the process data and the set value is utilized by the PID controller, to drive the pacing signal by determining the desired heart rate.

FIG. 53 illustrates a block diagram of cross-check validation scheme for generating reliable pacing signals. Each skin-interfaced device (cardiac, respiratory, hemodynamic modules) integrates in-sensor algorithms for heart rate (HR) and respiratory rate (RR) calculation and transmits raw data (cardiac module: 500 Hz, respiratory module: 1600 Hz, hemodynamic module: 100 Hz) to the control module along with processed real-time HR and RR data. The control module calculates real-time HR and RR locally and performs cross-checking validation with the transmitted HR and RR from the networked collection of skin-interfaced devices.

### DETAILED DESCRIPTION OF THE INVENTION

The invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this invention will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the invention has been specifically disclosed by preferred embodiments and optional features, modification and variation of the concepts herein disclosed may be resorted to by those skilled in the art, and that such modifications and variations are considered to be within the scope of this invention as defined by the appended claims.

Whenever a range is given in the specification, for example, a temperature range, a time range, or a composition or concentration range, all intermediate ranges and subranges, as well as all individual values included in the ranges given are intended to be included in the invention. It will be understood that any subranges or individual values in a range or subrange that are included in the description herein can be excluded from the claims herein.

It will be understood that, as used in the description herein and throughout the claims that follow, the meaning of “a”, “an”, and “the” includes plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation depicted in the figures. For example, if the device in one of the figures is turned over, elements described as being on the “lower” side of other elements would then be oriented on “upper” sides of the other elements. The exemplary term “lower”, can therefore, encompasses both an orientation of “lower” and “upper,” depending of the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

It will be further understood that the terms “comprises” and/or “comprising”, or “includes” and/or “including”, or “has” and/or “having”, or “carry” and/or “carrying”, or “contain” and/or “containing”, or “involve” and/or “involving”, “characterized by”, and the like are to be open-ended, i.e., to mean including but not limited to. When used in this disclosure, they specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the invention, and will not be interpreted in an idealized or overly formal sense unless expressly so defined herein.

As used in the disclosure, “around”, “about”, “approximately” or “substantially” shall generally mean within 20 percent, preferably within 10 percent, and more preferably within 5 percent of a given value or range. Numerical quantities given herein are approximate, meaning that the term “around”, “about”, “approximately” or “substantially” can be inferred if not expressly stated.

As used in the disclosure, the phrase “at least one of A, B, and C” should be construed to mean a logical (A or B or C), using a non-exclusive logical OR. As used herein, the term “and/or” includes any and all combinations of one or more of the associated listed items.

The term “flexibility” or “bendability”, as used in the disclosure, refers to the ability of a material, structure, device or device component to be deformed into a curved or bent shape without undergoing a transformation that introduces significant strain, such as strain characterizing the failure point of a material, structure, device or device component. In an exemplary embodiment, a flexible material, structure, device or device component may be deformed into a curved shape without introducing strain larger than or equal to 5%, for some applications larger than or equal to 1%, and for yet other applications larger than or equal to 0.5% in strain-sensitive regions. A used herein, some, but not necessarily all, flexible structures are also stretchable. A variety of properties provide flexible structures (e.g., device components) of the invention, including materials properties such as a low modulus, bending stiffness and flexural rigidity; physical dimensions such as small average thickness (e.g., less than 100 microns, optionally less than 10 microns and optionally less than 1 micron) and device geometries such as thin film and open or mesh geometries.

The term “bending stiffness” refers to a mechanical property of a material, device or layer describing the resistance of the material, device or layer to an applied bending moment. Generally, bending stiffness is defined as the product of the modulus and area moment of inertia of the material, device or layer. A material having an inhomogeneous bending stiffness may optionally be described in terms of a “bulk” or “average” bending stiffness for the entire layer of material.

The term “elastomer”, as used in the disclosure, refers to a polymeric material which can be stretched or deformed and return to its original shape without substantial permanent deformation. Elastomers commonly undergo substantially elastic deformations. Useful elastomers include those comprising polymers, copolymers, composite materials or mixtures of polymers and copolymers. Elastomeric layer refers to a layer comprising at least one elastomer. Elastomeric layers may also include dopants and other non-elastomeric materials. Useful elastomers useful include, but are not limited to, thermoplastic elastomers, styrenic materials, olefenic materials, polyolefin, polyurethane thermoplastic elastomers, polyamides, synthetic rubbers, PDMS, polybutadiene, polyisobutylene, poly(styrene-butadiene-styrene), polyurethanes, polychloroprene and silicones. Exemplary elastomers include, but are not limited to, silicon containing polymers such as polysiloxanes including poly(dimethyl siloxane) (i.e., PDMS and h-PDMS), poly(methyl siloxane), partially alkylated poly(methyl siloxane), poly(alkyl methyl siloxane) and poly(phenyl methyl siloxane), silicon modified elastomers, thermoplastic elastomers, styrenic materials, olefenic materials, polyolefin, polyurethane thermoplastic elastomers, polyamides, synthetic rubbers, polyisobutylene, poly(styrene-butadiene-styrene), polyurethanes, polychloroprene and silicones. In one embodiment, a flexible polymer is a flexible elastomer.

The term “encapsulate” or “encapsulation”, as used in the disclosure, refers to the orientation of one structure such that it is at least partially, and in some cases completely, surrounded by one or more other structures. “Partially encapsulated” refers to the orientation of one structure such that it is partially surrounded by one or more other structures. “Completely encapsulated” refers to the orientation of one structure such that it is completely surrounded by one or more other structures. The invention includes devices having partially or completely encapsulated electronic devices, device components and/or inorganic semiconductor components.

Embodiments of the invention are illustrated in detail hereinafter with reference to accompanying drawings. The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in different order (or concurrently) without altering the principles of the invention.

The current invention introduce a transient closed-loop system that combines a time-synchronized, wireless network of soft, skin-interfaced devices with an advanced bioresorbable pacemaker to control cardiac rhythms, track cardiopulmonary status, provide multi-haptic feedback, and enable transient operation with minimal patient burden. This system provides a range of robust, rate-adaptive cardiac pacing capabilities, as demonstrated in rat, canine, and human heart studies of autonomous treatment of bradycardias. This work establishes a generalizable engineering framework for closed-loop temporary electrotherapy using wirelessly linked, body-integrated bioelectronic devices.

Temporary postoperative cardiac pacing requires devices with percutaneous leads and external wired power and control systems. This hardware introduces risks for infection, limitations on patient mobility, and requirements for surgical extraction procedures. Bioresorbable pacemakers mitigate some of these disadvantages, but they demand pairing with external, wired systems and secondary mechanisms for control. We present a transient closed-loop system that combines a time-synchronized, wireless network of skin-integrated devices with an advanced bioresorbable pacemaker to control cardiac rhythms, track cardiopulmonary status, provide multi-haptic feedback, and enable transient operation with minimal patient burden. The result provides a range of autonomous, rate-adaptive cardiac pacing capabilities, as demonstrated in rat, canine, and human heart studies. This work establishes an engineering framework for closed-loop temporary electrotherapy using wirelessly linked, body-integrated bioelectronic devices.

In one aspect of the invention, the transient closed-loop system for cardiac pacing and/or defibrillator therapy for a subject, comprises a bioresorbable module configured to at least partially attach to an epicardial interface of the subject's heart for the cardiac pacing; at least one skin-interfaced module configured to attach to an outer surface of the subject's skin, wherein the bioresorbable module is in wireless communication with the at least one skin-interfaced module; and a control module in wireless communication with the at least one skin-interfaced module.

In one embodiment, the bioresorbable module dissolves in the subject's body after a period of time.

In one embodiment, the period of time is at least 10 days, 20 days or 30 days.

In one embodiment, the period of time is customizable.

In one embodiment, the control module is configured to calculate at least one regular heart rate and provide autonomous cardiac pacing to the subject according to the at least one regular heart rate.

In one embodiment, the at least one regular heart rate comprises a high rate limit and a low rate limit.

In one embodiment, when a heart rate of the subject is lower than the low rate limit, the control module activates the bioresorbable module to provide electrical stimulation to the heart at a pre-specified rate, for cardiac pacing.

In one embodiment, when the heart rate of the subject is higher than the high rate limit, the bioresorbable module remains inactive.

In one embodiment, the at least one skin-interfaced module comprises a cardiac module.

In one embodiment, the cardiac module is configured to operably place over skin of the subject's chest area.

In one embodiment, the at least one skin-interfaced module further comprises a respiration module in wireless communication with the control module.

In one embodiment, the respiration module is configured to operably collect physiological information of the subject and wirelessly transmit the physiological information to the control module.

In one embodiment, the control module operably calculates the at least one regular heart rate according to the physiological information collected by the respiration module and provides autonomous cardiac pacing to the subject according to the at least one regular heart rate.

In one embodiment, the at least one skin-interfaced module further comprises a hemodynamic module in wireless communication with the control module.

In one embodiment, the hemodynamic module is configured to operably collect physiological information of the subject and wirelessly transmit the physiological information to the control module.

In one embodiment, the control module operably calculates the at least one regular heart rate according to the physiological information collected by the hemodynamic module and provides autonomous cardiac pacing to the subject according to the at least one regular heart rate.

In one embodiment, the at least one skin-interfaced module further comprises a haptic module in wireless communication with the control module.

In one embodiment, the haptic module in configured to operably receive tactile information from the control module.

In one embodiment, the haptic module operably provides at least one pattern of vibro-tactile according to the tactile information received from the control module.

In one embodiment, the bioresorbable module comprises a power harvester configured to operably receive power delivery from the cardiac module; at least one stimulation electrode configured to operably deliver stimuli to the epicardial interface of the subject's heart for the cardiac pacing; and a stretchable interconnect connecting the power harvester and the stimulation electrode.

In one embodiment, the bioresorbable module is encapsulated by bioresorbable dynamic covalent polyurethane (b-DCPU), polylactic acid (PLA), polyglycolic acid (PGA), polyglycolide (PGL), polycaprolactone (PCL), poly(glycerol sebacate) (PGS), poly(octamethylenemaleate (anhydride) citrate)) (POMaC), poly(1,8-octanediol-co-citric acid) (POC), poly(butylene succinate) (PBS), poly(butylene adipate) (PBA), ureidopyrimidinone (Upy), poly(sebacoyl diglyceride) (PSeD-U), and/or Polybuthanedithiol 1,3,5-triallyl-1,3,5-triazine-2,4,6(1H,3H,5H)-trione pentenoic anhydride (PBTPA).

In one embodiment, the power harvester comprises at least one receiver (Rx) coil.

In one embodiment, the cardiac module comprises at least one transmission (Tx) coil.

In one embodiment, the Rx coil of the power harvester is wirelessly coupled to the Tx coil of the cardiac module via magnetic induction for receiving power delivery from the cardiac module.

In one embodiment, the stimulation electrode of the bioresorbable module is operably attached to the epicardial interface of the subject's heart, and the Rx coil of the power harvester is operably placed subcutaneously and in vicinity to the cardiac module.

In one embodiment, the Rx coil at least partially overlaps with the Tx coil and is placed within 25 mm of the Tx coil.

In one embodiment, the Rx coil operably receives the power delivery from a tethered wireless charger when the cardiac module is removed from the subject.

In one embodiment, the cardiac module comprises:
a transmission (Tx) coil configured to deliver power to the bioresorbable module; and
a wireless charging coil configured to receive power delivery from an external power source.

In one embodiment, the cardiac module further comprises a Bluetooth low energy (BLE) system-on-chip (SoC), an ECG analog front end (AFE), and/or an RF power amplifier.

In one embodiment, the stimulation electrode comprises an electrode that is dissolvable.

In one embodiment, the electrode operates for more than 30 days before being dissolved.

In one embodiment, the electrode, the Rx coil and the interconnects are formed of a bioresorbable conductor including molybdenum (Mo), zinc (Zn), iron (Fe), tungsten (W), magnesium (Mg), and/or AZ31B (3 wt % Al and 1 wt % Zn) Mg alloy.

In one embodiment, the stimulation electrode further comprises a bioresorbable steroid eluting patch.

In one embodiment, the bioresorbable steroid eluting patch is configured to operably reduce fibrotic tissue growth at an interface between the bioresorbable module and heart tissue.

In one embodiment, the cardiac module operably receives pacing information from the control module regarding the cardiac pacing of the subject's heart.

In one embodiment, the cardiac module operably delivers the pacing information to the bioresorbable module so as to control the cardiac pacing.

In one embodiment, the bioresorbable module operably provides a charge-balanced biphasic waveform.

In one embodiment, the bioresorbable module is stretchable, twistable, and bendable.

In one embodiment, the skin-interfaced module is stretchable, pristinable, and bendable.

In one embodiment, the skin-interfaced module is peelable from the skin of the subject.

In one embodiment, the control module comprises a hand-held terminal.

In one embodiment, the control module has an interactive interface for receiving and displaying information.

In one embodiment, the system operably provides the cardiac pacing for treatment of bradycardia.

In one embodiment, the system operably detects a heart rate of the subject and determines a heart condition based on the heart rate, and initiates the cardiac pacing without the subject's intervention.

In one embodiment, the system is MRI safe.

In another aspect of the invention, the transient closed-loop system for cardiac pacing and/or defibrillator therapy for a subject, comprises a bioresorbable module for cardiac pacing and/or defibrillator therapy; and a network of skin-integrated modules coupled with the bioresorbable module to control cardiac rhythms, track cardiopulmonary status, provide multi-haptic feedback, and enable transient operation with minimal patient burden.

In one embodiment, the bioresorbable module is configured to wirelessly receive power by inductive coupling to pace the subject's heart through an epicardial interface of the heart for epicardial pacing.

In one embodiment, the bioresorbable module comprises a bioresorbable, stretchable epicardial pacemaker operably attached to the epicardial interface; a bioresorbable steroid-eluting patch coupled to the pacemaker and configured to minimize local inflammation and fibrosis; and a bioresorbable power harvester coupled to the pacemaker to power the pacemaker.

In one embodiment, the pacemaker comprises at least one stimulation electrode connected to the power harvester via stretchable interconnects and configured to operably deliver stimuli to the epicardial interface of the subject's heart for the cardiac pacing.

In one embodiment, the power harvester comprises an antenna for delivering power to the pacemaker, wherein the antenna comprises a loop antenna having at least one coil.

In one embodiment, the power harvester further comprises at least one PIN diode electrically coupled between the antenna and the pacemaker.

In one embodiment, the stimulation electrode, the interconnects and the antenna are formed of a bioresorbable conductor including molybdenum (Mo), zinc (Zn), iron (Fe), tungsten (W), magnesium (Mg), and/or AZ31B (3 wt % Al and 1 wt % Zn) Mg alloy.

In one embodiment, the bioresorbable module further comprises top and bottom encapsulating layers formed of a bioresorbable dynamic covalent polyurethane (b-DCPU) that define a mechanically stretchable structure sealed by thermally activated dynamic bond exchange reactions. In another embodiment, the top and bottom encapsulating layers are formed of polylactic acid (PLA), polyglycolic acid (PGA), polyglycolide (PGL), polycaprolactone (PCL), poly(glycerol sebacate) (PGS), poly(octamethylenemaleate (anhydride) citrate)) (POMaC), poly(1,8-octanediol-co-citric acid) (POC), poly(butylene succinate) (PBS), poly(butylene adipate) (PBA), ureidopyrimidinone (Upy), poly(sebacoyl diglyceride) (PSeD-U), and/or Polybuthanedithiol 1,3,5-triallyl-1,3,5-triazine-2,4,6(1H,3H,5H)-trione pentenoic anhydride (PBTPA).

In one embodiment, the bioresorbable module is a fully implantable, bioresorbable module.

In one embodiment, the bioresorbable module dissolves in the subject's body after a period of time.

In one embodiment, the network of skin-integrated modules comprises a set of flexible, skin-interfaced sensors placed on various locations of the body and configured to capture physiological monitoring of the subject, wherein the physiological information comprises electrocardiograms (ECGs), heart rate (HR), respiratory information, physical activity, and/or cerebral hemodynamics; a radiofrequency (RF) module configured to wirelessly transfer the power from an external power source to the power harvester; and a flexible, skin-interfaced haptic actuator configured to communicate via mechanical vibrations.

In one embodiment, the set of flexible, skin-interfaced sensors comprises at least one respiration module, and/or at least one hemodynamic module.

In one embodiment, the radiofrequency (RF) module comprises a cardiac module comprising a wireless charging unit configured to receive the power charged from an external power source; and a transmission (Tx) coil configured to wirelessly transmit the power to the power harvester.

In one embodiment, the system further comprises a control module in wireless communication with the network of skin-interfaced modules for receiving information from the skin-interfaced modules and controlling the skin-interfaced modules.

In one embodiment, the control module comprises a portable device with a software application for real-time visualization, storage, and analysis of data for automated adaptive control.

T In one embodiment, the skin-interfaced haptic actuator comprises a haptic module configured to wirelessly receive tactile information, and provide at least one pattern of vibro-tactile according to the tactile information.

In one embodiment, the control module operably calculates the at least one regular heart rate according to the physiological information collected by the respiration module and the hemodynamic module, and provides autonomous and wireless pacing therapy to the subject according to the at least one regular heart rate.

In one embodiment, the at least one regular heart rate comprises a high rate limit and a low rate limit.

In one embodiment, when a heart rate of the subject is lower than the low rate limit, the control module activates the bioresorbable module to provide electrical stimulation to the heart at a pre-specified rate, for autonomous and wireless pacing therapy.

In one embodiment, when the heart rate of the subject is higher than the high rate limit, the bioresorbable module remains inactive.

In one embodiment, the control module processes real-time HR and respiratory rate locally and performs cross-checking validation with the transmitted HR and respiratory rate values from networked collection of the skin-interfaced modules.

In one aspect, the invention relates to a method for installing a transient closed-loop system for cardiac pacing and/or defibrillator therapy for a subject. The method comprises coupling at least a part of a bioresorable module to an epicardial interface of the subject's heart for the cardiac pacing; and attaching at least one skin-interfaced module to an outer surface of the subject's skin, wherein the bioresorbable module is in wireless communication with the at least one skin-interfaced module; and a control module is in wireless communication with the at least one skin-interfaced module.

In one embodiment, the at least one skin-interfaced module comprising a cardiac module.

In one embodiment, the cardiac module is placed over skin of the subject's chest area.

In one embodiment, the at least one skin-interfaced module further comprises a hemodynamic module in wireless communication with the control module; and wherein the method further comprises attaching the hemodynamic module to the skin outer surface of the subject.

In one embodiment, the at least one skin-interfaced module further comprises a respiration module in wireless communication with the control module; and wherein the method further comprises attaching the respiration module to the skin outer surface of the subject.

In one embodiment, the at least one skin-interfaced module further comprises a haptic module in wireless communication with the control module; and wherein the method further comprises attaching the haptic module to the skin outer surface of the subject.

In one embodiment, the skin-interfaced module is peelable from the skin of the subject.

In one embodiment, the bioresorbable module dissolves after a period of time.

In one embodiment, the period of time is at least 10 days, 20 days, 30 days, or customizable.

In one embodiment, the bioresorbable module is encapsulated by bioresorbable dynamic covalent polyurethane (b-DCPU), polylactic acid (PLA), polyglycolic acid (PGA), polyglycolide (PGL), polycaprolactone (PCL), poly(glycerol sebacate) (PGS), poly(octamethylenemaleate (anhydride) citrate)) (POMaC), poly(1,8-octanediol-co-citric acid) (POC), poly(butylene succinate) (PBS), poly(butylene adipate) (PBA), ureidopyrimidinone (Upy), poly(sebacoyl diglyceride) (PSeD-U), and/or Polybuthanedithiol 1,3,5-triallyl-1,3,5-triazine-2,4,6(1H,3H,5H)-trione pentenoic anhydride (PBTPA).

In one embodiment, the bioresorbable module comprises a power harvester configured to operably receive power delivery from the cardiac module; a stimulation electrode configured to operably deliver stimulus to the epicardial interface of the subject's heart for the cardiac pacing; and a stretchable interconnect connecting the power harvester and the stimulation electrode.

In one embodiment, the power harvester comprises at least one receiver (Rx) coil.

In one embodiment, the cardiac module comprises:
a transmission (Tx) coil for delivering power to the bioresorbable module; and a wireless charging coil configured to receive power delivery from an external power source.

In one embodiment, the method further comprises wirelessly coupling the Rx coil of the power harvester to the Tx coil of the cardiac module such that the bioresorbable module receives power delivery from the Tx coil of the cardiac module via magnetic induction.

In one embodiment, the stretchable electrode of the bioresorbable module is attached to a surface area of the subject, and the Rx coil of the power harvester is placed subcutaneously and in vicinity to the cardiac module.

In one embodiment, the Rx coil of the power harvester receives power delivery from a tethered wireless charger when the cardiac module is removed from the subject.

In one embodiment, the stimulation electrode comprises an electrode that is dissolvable.

In one embodiment, the electrode is formed of a bioresorbable conductor including molybdenum (Mo), zinc (Zn), iron (Fe), tungsten (W), magnesium (Mg), and/or AZ31B (3 wt % Al and 1 wt % Zn) Mg alloy.

In one embodiment, the stimulation electrode further comprising a bioresorbable steroid eluting patch.

In one embodiment, the bioresorbable steroid eluting patch is configured to operably reduce the fibrotic tissue growth at the interface between the bioresorbable module and epicardial interface.

In one embodiment, the control module comprises a hand-held terminal.

In one embodiment, the control module has an interactive interface for receiving and displaying information.

In another aspect, the invention relates to a method of cardiac pacing and/or defibrillator therapy for a subject with a transient closed-loop system having a bioresorable module, at least one skin-interfaced module and a control module. The method comprises wirelessly transmitting at least one parameter of cardiac pacing from the control module to the at least one skin-interfaced module; wirelessly transmitting the at least one parameter from the at least one skin-interfaced module to the bioresobable module; and pacing the subject's heart by the bioresorbable module according to the at least one parameter.

In one embodiment, the at least one skin-interfaced module comprising a cardiac module placed over skin of the subject's chest area.

In one embodiment, the at least one skin-interfaced module further comprising a respiration module in wireless communication with the control module.

In one embodiment, the at least one skin-interfaced module further comprising a hemodynamic module in wireless communication with the control module.

In one embodiment, the at least one skin-interfaced module further comprising a haptic module in wireless communication with the control module.

In one embodiment, the method further comprises transmitting haptic information to the haptic module by the control module.

In one embodiment, the haptic information comprises at least one pattern of vibro-tactile.

In one embodiment, the haptic module vibrates according to the pattern of vibro-tactile received.

In one embodiment, the wireless communication between the control module and the cardiac module is via a Bluetooth low energy (BLE) protocol.

In one embodiment, the wireless communication between the cardiac module and the bioresorbable module is via at least one of a Bluetooth low energy (BLE) protocol and a near field communication (NFC) protocol.

In one embodiment, the method further comprises collecting hemodynamic physiological information of the subject by the hemodynamic module; and wirelessly transmitting the hemodynamic physiological information to the control module.

In one embodiment, the method further comprises collecting respiration physiological information of the subject by the respiration module; and wirelessly transmitting the respiration physiological information to the control module.

In one embodiment, the method further comprises calculating at least one regular heart rate by the control module based on the physiological information received; adjusting the at least one parameter for cardiac pacing by the control unit based on the physiological information; and providing the at least one parameter to the cardiac module by the control unit.

In one embodiment, the method further comprises wirelessly transmitting the at least one parameter from the cardiac module to the bioresobable module; and pacing the subject's heart by the bioresorbable module according to the at least one parameter.

In one embodiment, the at least one regular heart rate comprising a high rate limit and a low rate limit.

In one embodiment, when a heart rate of the subject detected by the cardiac module is lower than the low rate limits, the system activates the bioresorbable module, to provide electrical stimulation to the heart at a pre-specified rate.

In one embodiment, when the heart rate of the subject detected by the system is higher than the high rate limits, the bioresorbable module remains inactive.

In one embodiment, the control module calculates at least one regular heart rate according to the respiration and hemodynamic information collected by the respiration module and the hemodynamic module and provides autonomous cardiac pacing to the subject according to the at least one regular heart rate.

In one embodiment, the method further comprises initiating the cardiac pacing without the subject's intervention.

In one embodiment, the control module processes real-time HR and respiratory rate locally and performs cross-checking validation with the transmitted HR and respiratory rate values from networked collection of skin-interfaced modules including the cardiac, respiratory, and hemodynamic modules.

External and internal electrical pacing of the heart are fundamental interventions in patients with cardiovascular disease. This invention, among other things, discloses a bioresorbable closed-loop sensor-actuator system capable of controlling heart function in patients with a postsurgical risk of bradycardia (slow heart rate). This technology is wireless, circumventing common shortcomings of implanted devices, such as drive-line infections or the need for surgical procedures to remove or replace, for example, pacemaker leads or batteries. The demonstrated cardiac application of this technology in rats, dogs, and ex vivo human heart preparations could improve outpatient surveillance, allowing for earlier release from the hospital and remote monitoring of patients living in medically underserved areas.

This invention makes use of a previously introduced design strategy, comprising water soluble metals (molybdenum and silicon) and degradable polymers (polyurethane and poly(lactic-co-glycolic acid)), to fabricate resorbable devices. In addition to providing preclinical proof of concept or the use in monitoring and pacing the heart, we demonstrated important additional features of the device such as magnetic resonance imaging (MRI) compatibility, mechanical robustness, rechargeability, rate-adapted pacing capabilities and secure data processing.

Triggering electrical pulsing in response to sensing defined biosignals adds a new layer of complexity to human-machine interfacing. The technology described in the disclosure could have broad applications in sensing and controlling the function not only of the heart but also of other excitable organs or tissue. In some cases, transient support will be desirable and sufficient, such as in patients with transient paralysis. There may be other scenarios where chronic or even permanent use may be of interest, such as in patients with a dysfunctional pacing function of the sinus node (sick sinus syndrome) or permanent paralysis after, for example, traumatic denervation.

These and other aspects of the invention are further described below. Without intent to limit the scope of the invention, exemplary instruments, apparatus, methods and their related results according to the embodiments of the invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### Example 1

### A Transient, Closed-Loop Network of Wireless, Body-Integrated Devices for Autonomous Electrotherapy

Temporary postoperative cardiac pacing requires devices with percutaneous leads and external wired power and control systems. This hardware introduces risks for infection, limitations on patient mobility, and requirements for surgical extraction procedures. Bioresorbable pacemakers mitigate some of these disadvantages, but they demand pairing with external, wired systems and secondary mechanisms for control.

In this exemplary study, we present a transient closed-loop system that combines a time-synchronized, wireless network of skin-integrated devices with an advanced bioresorbable pacemaker to control cardiac rhythms, track cardiopulmonary status, provide multi-haptic feedback, and enable transient operation with minimal patient burden. The result provides a range of autonomous, rate-adaptive cardiac pacing capabilities, as demonstrated in rat, canine, and human heart studies. This work establishes an engineering framework for closed-loop temporary electrotherapy using wirelessly linked, body-integrated bioelectronic devices.

### Materials and Methods

Preparation of Bioresorbable Module: Bioresorbable metal foils of Mo (10 μm thick; Goodfellow) for the bioresorbable module and W/Mg foils for the reference module were attached on the b-DCPU substrate (called bottom substrate, about 100 μm thick) using the intrinsic tackiness of the surface of this material. Sputter coating formed a layer of W (700 nm thick) on a Mg foil (50 μm thick; Solution Materials) to achieve high contrast CT images for non-invasive monitoring of bioresorption processes. Improved adhesion follows from pre-treating both metal foils and b-DCPU by exposure to oxygen plasma (200 mTorr, 200 W, 120 s; Reactive Ion Etch Plasma System, Nordson March, CA, USA). Laser-cutting the Mo (10 μm thick; Goodfellow) or W/Mg foils attached on the b-DCPU (about 100 μm thick) defined a receiving inductive (Rx) coil and extension electrode structures. The PIN diode was transferred onto a bottom b-DCPU substrate. Covering the components with b-DCPU (top substrate) and hot pressing the entire system yielded a compact, double-coil structure with openings for interconnects. All electrical components were interconnected with a biodegradable conductive W paste. The steroid eluting patch was inserted through the hole on the bottom PU substrate. Remaining holes were encapsulated with bioresorbable hydrophobic polyanhydride. Following is the geometry of the bioresorbable module with 12 mm Rx coil: width, 14.5 mm; length, less than 40 mm; thickness, about 250 μm; weight, 0.3 g.

Synthesis of Steroid Eluting Patch: Poly(D,L-lactide-co-glycolide) (PLGA) 50:50 (lactide:glycolide 50:50, mol wt 30,000-60,000), PLGA 65:35 (lactide:glycolide 65:35, mol wt 40,000-75,000), PLGA 75:25 (lactide:glycolide 75:25, mol wt 66,000-107,000), dexamethasone acetate (DMA), and anhydrous ethyl acetate were obtained from Sigma Aldrich. The synthesis involved dissolving 0.2 g/mL PLGA of each lactide:glycolide ratio (50:50, 65:35, 75:25) and 9.6 mg/mL DMA in ethyl acetate. The solution was stirred for 24 h at room temperature to disperse the dexamethasone acetate in solution. After mixing, the solution was drop cast onto a 4-inch silicon wafer vacuum treated with trichloro(1H,1H,2H,2H-perfluoroctyl) silane (Sigma-Aldrich, USA) to yield films with thicknesses defined by the volume and area. The films were allowed to dry in a desiccator with a small outlet for 12 h to yield semi-transparent steroid eluting films. The films were laser cut to their final dimensions (4.64×3.34 mm²) for the device. The mass of the drug in the steroid eluting patch is calculated as follows:

$$drug mass in one patch = total drug mass \times \frac{mass of patch}{total film mass}$$

Control samples were synthesized using the same concentration of PLGA according to the above procedure.

Design and Components for the Skin-Interfaced Cardiac Module: Schematic diagrams for the fPCB and the board layout were designed using AUTODESK EAGLE (version 9.6.0). Serpentine-shaped features between three separate islands (main body, charging coil, pacing coil) formed reliable electrical interconnections with a high level of deformability and with mechanical isolation of the islands. The components include 0201 and 0402 inch footprint passive elements (resistors, capacitors, and inductors), four turn coils for wireless charging and powering (resonant frequency: 13.56 MHz), a full-bridge rectifier, a power management IC (STBC15, STMicroelectronics), a 3.3V output power management IC (XC6206P332MR-G, Torex Semiconductor Ltd), a 3.6V output power management IC (XC6206P362MR-G, Torex Semiconductor Ltd), a 3.7V lithium polymer battery (80 mAh), a voltage and current protection IC for the battery (BQ2970, Texas Instruments), a single-lead ECG front end (AD8232, Analog devices), a Bluetooth SoC (nRF52832, Nordic Semiconductor), a single inverter (NC7SZ14, On Semiconductor) and an enhancement mode MOSFET (ZXMN6A07Z, Zetex).

Fabrication and Encapsulation of the Skin-Interfaced Cardiac Module: Panels of fPCB manufactured by an ISO 9001-compliant vendor, were folded into the final geometry and the battery was soldered into place. Customized firmware was downloaded by Segger Embedded Studio. The aluminum molds for the top and bottom encapsulating layers of silicone elastomer (Silbione-4420, each 300 μm thick) were prepared with a freeform prototyping machine (Roland MDX 540). After pouring the 1:1 mixture of silicone elastomer and squeezing the top/bottom molds together with clamps, baking at 95° C. in an oven for 20 min cured the mixture. After 20 min, the mold was removed from the oven and placed on the room temperature area for 20 min to cool. The fPCB was encapsulated between these top and bottom layers after pouring with another silicone elastomer (Eco-Flex 0030, 1:1 ratio) into a cavity where the device was positioned on the mold. After squeezing the molds with clamps, curing in an oven at 65° C. for 30 min completed the process. Following is the geometery of the skin-interfaced cardiac module: width, 50 mm; length, 35 mm; thickness, <8 mm; weight, 7.3 g.

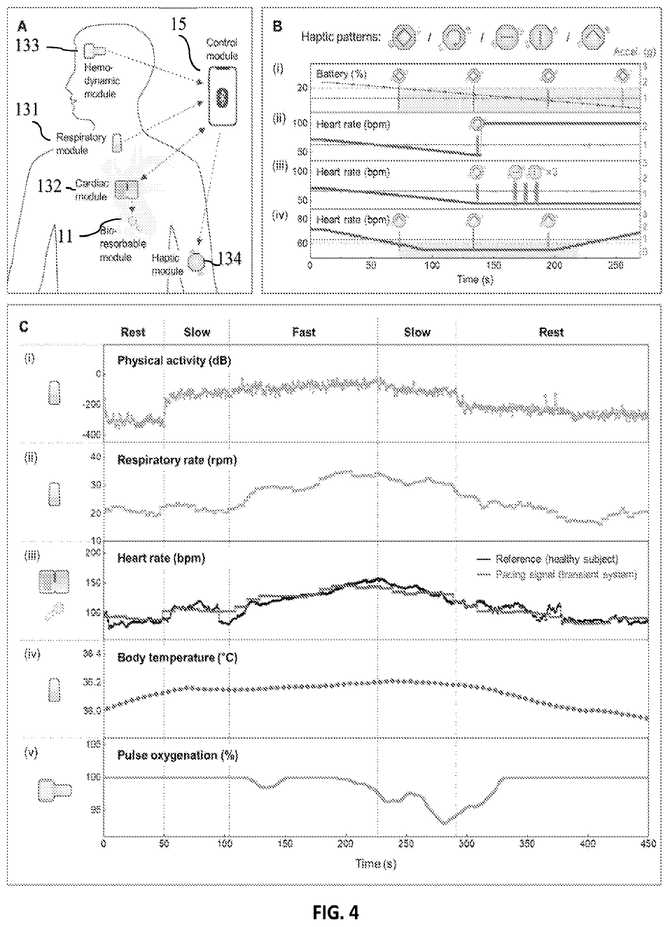

Design and Fabrication of the Skin-Interfaced Multi-Haptic Module: The multi-haptic module includes Bluetooth communication components (CC2640, Texas Instruments, 2450AT18D0100, Johanson Tech.), a microcontroller (ATMega328P, Microchip Technology), a collection of eccentric rotating mass (ERM; CLP0820B004L, Jinlong Machinery & Electronics Co. Ltd) actuators, and NFC charging components mounted on a fPCB designed with serpentine structures that optimize for overall mechanical flexibility of the system. A pulse-width modulated (PWM) signal from the microcontroller defines the operation of the ERM. The NFC charging system exploits a coil with three turns formed on the fPCB (resonant frequency: 13.56 MHz), a full-bridge rectifier and a DC-DC converter to charge the 3.7V lithium polymer battery (45 mAh). The four ERM actuators were individually controlled to five different power levels and at a period of 30 ms, to deliver diverse haptic feedback patterns to the skin. The device was encapsulated with a top and bottom silicone elastomer layer (Silbione-4420). Following is the geometry of the skin-interfaced multi-haptic module: size, 45×45 mm²; thickness, 6.4 mm; weight, 6.5 g. Following are the setting parameters for the demonstration of the patient awareness function using the multi-haptic module shown in FIG. 4. (i) Alarm for low battery condition of the cardiac module, with a threshold value of 20% of battery capacity. Interval (time between vibration), 0.5 s. Repetition, vibrating every 1 min until device replacement. The red region indicates low-battery status. (ii) Indicator for initiating pacing to treat bradycardia. Setting value (bradycardia), 45 bpm. No interval (continuous). No repetition (one time alarm). (iii) Alarm for pacemaker malfunction. Interval, 0.5 s. Repetition, every 5 second, 3 times. (iv) Indicator for bradycardia, with a threshold value of 60 bpm. Interval, 0.5 s. Repetition, continuous ever 1 min. until detecting normal heart rate.

Sensor Assessment and Human Subject Studies: The studies were approved by Northwestern University Institutional Review Board (NU-526 IRB), Chicago, IL, USA (STU00202449 and STU00212522) and was registered on 527 ClinicalTrials.gov (NCT02865070, NCT04393558). The skin-interfaced cardiac and respiratory modules were secured on the chest and suprasternal notch of each subject using a double-sided hydrogel adhesive (KM 40A, KATECHO), respectively. The bioresorbable module was attached on the cardiac module and the pacing signal was monitored by the oscilloscope. Real-time ECG was captured using a reference device (NicoletOne, Natus) with three ECG electrodes (Red dot 2238.3M) attached on the left chest, right chest, and left abdomen. In addition, the respiratory rate was monitored by an FDA-approved portable real-time capnography device (EMMA, Masimo). ECG, respiratory rate, and the pacing output data were collected while the subject was at rest for 1 min, pedaling on a stationary bicycle in a low gear (level 1) for 1 min, pedaling in a high gear (level 6) for 2 min, pedaling in a low gear for 1 min, and at rest for 2 min. Data collection from the skin-interfaced modules and the reference devices started simultaneously with synchronized timestamps. From the capnography device, respiratory rate is displayed as breaths per minute, recorded after two breaths and updated every breath. In terms of electrical input protection, all terminals of the skin-interfaced device are protected against electrostatic discharge (ESD). External series resistors (10 MΩ) in series with each of the input limit current for voltages beyond the circuit supply. In this scenario, the skin-interfaced device can safely handle a continuous 5 mA current at room temperature and the current flow never exceeds 10 μA. In terms of maximum ratings for electrical characteristics, the analog front end (AFE) system on a chip (SoC) presents an 8 kV human body model ESD rating.

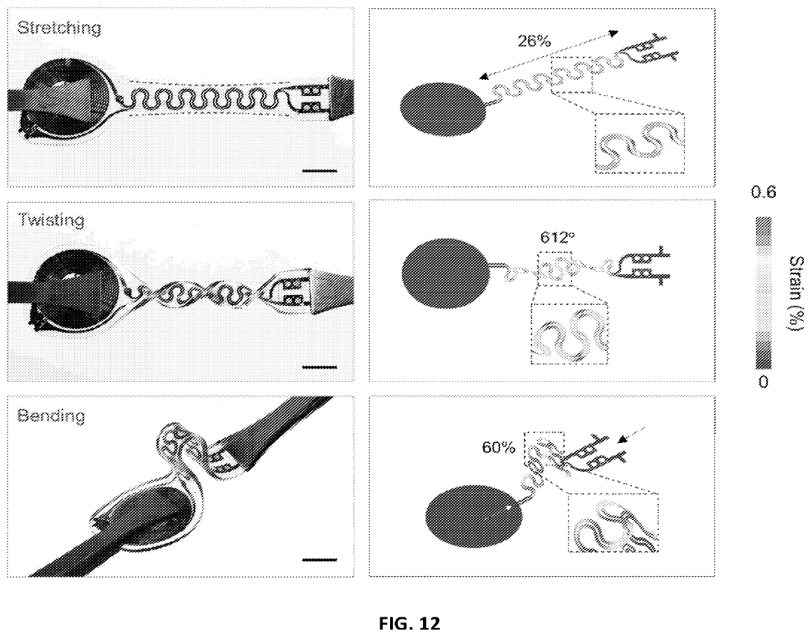

Simulation of the Mechanical Characteristics: The mechanical performance of the pacemaker serpentine interconnects under stretching, twisting, and compression was modeled using the commercial finite element analysis (FEA) software (ABAQUS, Analysis User's Manual 2016) to showcase the flexible and stretchable performance of the Mg electrode when subjected to the motions, and deformations, while in the surface of the heart. The strain the metallic layer remains below the yield strain (0.6%) for about 26% uniaxial stretching, a twisting angle of 612°, and about 60% compression as shown in FIG. 12. The b-DCPU encapsulation and thin Mo layer (10 μm) in the coil and electrodes were modeled by hexahedron elements (C3D8R) and shell elements (S4R), respectively. A mesh convergence study was performed by locally refining the mesh density around the serpentine metallic layer to ensure accuracy in the results; the total number of elements in the model is 663,080. Similarly, the strains in the encapsulation and electronic circuit of the skin-interfaced controller were quantified for uniaxial stretching and bending type physiological deformations present in the chest area to ensure that the device provides conformal operation without yielding the metal in electronic circuit. The encapsulation and electronics circuit were modeled by hexahedron elements (C3D8R) and shell elements (S4R), respectively. Local mesh density refinement was used to ensure mesh convergence and accuracy. The total number of elements in the model is 1,132,060. The device can undergo 17% uniaxial stretching and supports a convex bending radius of 2.2 mm and concave bending radius of 3.1 mm before the yield strain (0.3%) for copper is reached. The elastic modulus (E) and Poisson's ratio (v) used in the FEA simulation were E_{Mo}=330 GPa, v_{Mo}=0.29, E_{PU}=0.8 MPa, and v_{PU}=0.5, E_{PI}=2.5 GPa, v_{PI}=0.34, E_{Cu}=119 GPa, v_{Cu}=0.34, E_{Ecoflex}=60 kPa, and v_{Ecoflex}=0.5.

MRI Compatibility Test: Tests of the MRI compatibility of the implanted device used a bone-in skin-on chicken thigh. Measurements using a network analyzer (E5071C ENA series, Agilent Technologies, Santa Clara, CA) with a coil resonator tuned to 400 MHz confirmed that the bioresorbable module and skin-interfaced module do not resonate at 400 MHz. The bioresorbable module was then placed between the skin and muscle tissue to mimic subcutaneous implantation in a patient. The skin-interfaced module (i.e., transmitter) was placed on top of the skin to test device operation, and subsequently removed for MR imaging. The thigh was wrapped in plastic and positioned in the MR scanning bed.

MRI was performed on a Bruker Biospec 9430 (Bruker Biospin Inc, Billerica, MA, USA) using a 72 mm linear transmit-receive volume coil mounted in the center of the bore. After centering the sample in the magnet, the coil was tuned and matched, and localizer scans were acquired. Axial and sagittal images of the sample were acquired using a multi-echo spin echo pulse sequence (RARE) with TR/TE=4000/31.2 ms, RARE factor 8, 15 or 21 slices as needed for sample coverage, 2 mm slice thickness, 256×256 matrix, and 58 mm field of view. This process was followed by an accelerated spin echo imaging sequence (TurboRARE) to assess device heating in the presence of a scan with high energy deposition. Axial and coronal images were acquired using this sequence with TR/TE=2000/19.6, RARE factor 8, 9 or 21 slices as needed for sample coverage, 2 mm slice thickness, 256×256 matrix, and 60 mm field of view.

At the conclusion of imaging, the sample was removed from the magnet and assessed for heating in the vicinity of the bioresorbable module. The module was removed from the chicken and a temperature probe (PhysiTemp 7001H, Physitemp Instruments LLC, Clifton, NJ, USA) was placed between the skin and meat where the device coil had been located, and the temperature recorded. For comparison, the temperature was recorded in a second location approximately 4 cm away from the device.

Electromagnetic Simulation: The electromagnetic properties of the coils and wireless power transfer system were modeled using the commercial software package ANSYS HFSS to determine the individual coil inductances, scattering parameters S₁₁, and S₂₁ related to the power transmission, and the specific absorption rate (SAR) to ensure that the energy absorbed by the tissue is below the safety threshold. Lumped ports were used to obtain the scattering parameters Sₙₘ and port impedances Zₙₘ of the 12-, 18-, and 25-mm outer diameter Rx coils and the skin-interfaced Tx inductive coil. The inductance (L) and quality factor (Q), were obtained for all coils as Lₙ=Im{Zₙₙ}/(2πf) and Qₙ=|Im{Zₙₙ}/Re{Zₙₙ}|, where Re{Zₙₙ}, Im{Zₙₙ}, and f represent the real and imaginary parts of Zₙₘ, and the working frequency, respectively. The efficiency was obtained from η=|S₂₁|²×100% as a function of the separation distance between the coils for both the open-circuit case and by adding external resistors 1k-1M Ohm.

The wireless power transfer was modeled in air and on the heart to account for dielectric effects. For the tissue model, the 25 mm coil was placed at the interface of skin/heart tissues of dimensions 9 cm, 13 cm, and 8.5 cm (W,L,H) to approximate the size of a human adult heart and the average SAR was calculated as

$$SAR = \frac{σ {\vert E \vert}^{2}}{2 ρ} ,$$

where σ is the conductivity of the tissue, E is the root mean square of induced electric field and ρ is tissue density. For continuous pacing, an external coil with dimensions 15 cm×20 cm with an input power of 2 W was placed directly above the skin layer. For long-term pacing in small animals, a double loop Tx coil (AWG 12) generated a uniform magnetic field inside a 26 cm, 47 cm, 21 cm (W, L, H) where a simplified mouse mesh ellipsoid body with major (half) axes 8, 14, and 52 mm included the implantable the bioresorbable module and the SAR was calculated on the mouse body. For system configurations, the SAR is well below the safety guidelines of RF exposure. For all cases, an adaptive mesh (tetrahedron elements) and a spherical radiation boundary (radius 1000 mm) were adopted to ensure computational accuracy. The HFSS material library properties were used for the Mo and Cu conductors in the bioresorbable module, skin-interfaced module, cage coil, and external tethered coil. For biological tissues, the dielectric constant (ε), electrical conductivity (σ) and density (ϕ are: ε_{Skin}=295, σ_{Skin}=0.23 S/m, and ρ_{Skin}=1020 kg/m³ for the skin layer; ε_{Heart}=246, σ_{Heart}=0.53 S/m, and σ_{Heart}=1055 kg/m³ for the heart tissue; and ε_{Mouse}=40, σ_{Mouse}=0.5 S/m, and ρ_{Mouse}=1000 kg/m³ for the equivalent mouse body.

Simulations of Electromagnetics Associated with MRI: The finite element method was used in the commercial software ANSYS electronics desktop (HFSS 2021 R2) to determine the magnetic field and gradients of the magnetic field for a MRI procedure at a working frequency of 128 MHz. A Helmholtz coil was used to create a strong and uniform magnetic field B and simulate the MRI procedure by placing the device in the uniform magnetic field. An adaptive meshing technique (tetrahedron elements) was used along with a spherical radiation boundary of 1000 mm radius to ensure computational accuracy. The in-plane gradient of the magnetic field density at the interface between the device and heart tissue was calculated from

$$\vert {∇}_{p} B \vert = {[ {( \frac{\partial B}{\partial x} )}^{2} + {( \frac{\partial B}{\partial y} )}^{2} ]}^{\frac{1}{2}}$$

and the out-of-plane gradient was calculated from

$$\vert {∇}_{z} B \vert = \vert \frac{\partial B}{\partial z} \vert$$

where x, y, and z are the orthogonal coordinates of the device plane.

Simulations for Thermal Load during MRI Scan: The commercial software ABAQUS (ABAQUS Analysis 2016) was used to study the heat transfer process and temperature distribution on the skin after a one-time MRI scan with an oscillating magnetic field density of B=20 μT and a working time of 0.5 ms. The convective heat transfer coefficient of air was set to 6 W·m⁻²·K⁻¹ to account for free convection with the surrounding environment. Local mesh density refinement around the area of the device was used to ensure mesh convergence. The total number of heat transfer elements (DC3D10) used in the model is about 1,607,000. The thermal conductivity, heat capacity, and density used in the simulation are 138 W·m⁻¹·K⁻¹, 250 J·kg⁻¹·K⁻¹, and 10,200 kg·m⁻³ for Mo; 0.56 W·m⁻¹·K⁻¹, 3686 J·kg⁻¹·K⁻¹, and 1081 kg·m⁻³ for the heat tissue, and 0.12 W·m⁻¹·K⁻¹, 1400 J·kg⁻¹·K⁻¹, and 970 kg·m⁻³ for b-DCPU.

Study of Drug Releasing Behaviors of the Steroid Eluting Patches: Dulbecco's Phosphate Buffered Saline (DPBS) was obtained from Sigma Aldrich. Steroid eluting patches were weighed, immersed in DPBS in a quartz UV-vis cuvette and then sealed and stored at 37° C. between measurements. The releasing behavior of dexamethasone acetate was studied using UV-vis spectrophotometry (Perkin Elmer LAMBDA 1050). The absorbance at 242 nm, a characteristic peak for dexamethasone acetate, was normalized to the estimated mass of drug in the steroid-eluting patch, calculated by taking the ratio of the pad mass to the total mass of the sample multiplied by the total amount of drug added. In summary the calculation is as follows.

$$\% mass released = \frac{mass calculated from UV absorbance}{\frac{mass of patch}{total film mass} \times total drug mass}$$

Measurements were performed every 3-4 days for a total of 60 days.

In Vivo Studies Using Rat Models: All procedures were performed according to protocols (A364) approved by The George Washington University Institutional Animal Care and Use Committee (IACUC) and conforming with guidance from the Guide for Care and Use of Laboratory Animals published by the National Institutes of Health (NIH). Device implantation tests comply with the industry standard of biological testing of implantable medical devices (ISO 10993). Adult male and female Sprague-Dawley rats (Hilltop Animals, Scottsdale, PA) were used. General anesthesia was administered throughout the procedure using inhaled isoflurane vapors (2-3%) at 2 mL/min oxygen flow with an EZ anesthesia machine (EZ Systems Inc., EZ-SA 800). Ventilation was provided by the VentElite small animal ventilator (Harvard Apparatus, Holliston, MA). The heart was exposed via left thoracotomy. The electrodes of the bioresorbable module were implanted on the epicardium of the left ventricle using non-absorbable monofilament 6-0 polypropylene suture (Ethicon, 8705H). The Rx coil was placed in a subcutaneous pocket on the ventral or dorsolateral surface of the rat. The thoracic cavity, muscle, and skin were subsequently closed. At the end of the procedure, the bioresorbable modules were tested using ECG to verify their function and to confirm adequate attachment. Animals were extubated when a gentle toe pinch elicited a response and were monitored until sternal recumbency was regained. Appropriate post-operative monitoring and care were provided following surgery. For analgesia, a subcutaneous injection of buprenorphine (0.5-1.0 mg/kg) was administered before incision and once every 12 hours for 48 hours post-operation.

Induction of Bradycardia in an In Vivo Rat Model Using Propranolol: All procedures were performed according to protocols (A364) approved by The George Washington University Institutional Animal Care and Use Committee (IACUC). Following implantation of a bioresorbable module, the rat was anesthetized using inhaled isoflurane vapors (2.5%) at 2 mL/min oxygen flow with an EZ anesthesia machine (EZ Systems Inc., EZ-SA 800). Throughout the experiment, a three-lead ECG was monitored using a PowerLab data acquisition system with the LabChart software (ADInstruments, Sydney, Australia). Subdermal needle electrodes were placed in the Lead I configuration (positive electrode on the right forelimb, negative electrode on the left forelimb, ground electrode on the hindlimb). In addition, the skin-interfaced controller was placed on top of the skin at the location of the Rx coil. The controller was secured in place with a custom vest. After loss of consciousness was confirmed, an intraperitoneal injection of propranolol (1 mg/kg) was administered to induce bradycardia. Once the heart rate fell below the lower rate limit of the controller algorithm, gentle heat was applied to the animal by a heat lamp. Once the heart rate returned to the original baseline level, the heat lamp and anesthesia were removed. The animal was monitored until consciousness was regained.

ECG Recordings and Optical Mapping of Human Whole Heart Cardiac Pacing: All tissue procurement, preparation, and experiments were performed according to protocols approved by the Institutional Review Board (IRB) of The George Washington University and international guidelines for human welfare. De-identified donor human hearts rejected for organ transplant were acquired from the Washington Regional Transplant Community (WRTC, Falls Church, VA). The proximal aorta and coronary arteries were cannulated and perfused with cold University of Wisconsin cardioplegic solution. The heart was then transferred to a Langendorff-perfusion system with a custom tissue chamber. The heart was perfused with a modified Tyrode's solution (128.2 mM NaCl, 4.7 mM KCl, 1.05 mM MgCl₂, 1.3 mM CaCl₂, 1.19 mM NaH₂PO₄, 20 mM NaHCO₃, 11.1 mM glucose) and bubbled with 95% O₂/5% CO₂. The pressure of the heart was maintained between 60-80 mmHg. The perfusion system was maintained at 37° C. throughout the experiment. The electrode of the bioresorbable module was sutured to the ventricles. Positive and negative needle electrodes were placed in the ventricles with the ground electrode placed in the tissue chamber away from the heart for both the skin-interfaced controller and the PowerLab ECG acquisition system (ADInstruments, Sydney, Australia). Spatiotemporal dynamics of the activation of the transmembrane potential were recorded by optical mapping. In brief, the optical mapping methods are as follows: Mechanical motion of the heart was arrested using the electromechanical uncoupler blebbistatin (5-10 μM). The tissue was stained with di-4-ANDBQBS (125 nM), a voltage-sensitive fluorescent dye, to optically map voltage changes in the membrane potential up to 4 mm deep. Signals were recorded at 1 kHz using a 100×100 pixel high-speed CMOS camera with a MICAM05 acquisition system (SciMedia, Costa Mesa, CA).

Processing of Data From Optical Mapping: Optical signals were processed using an open-source custom MATLAB software. Each pixel was spatially filtered with a 7×7 uniform average bin. A Finite Impulse Response filter was used to filter each temporal sequence with a cutoff frequency of 75 Hz. Baseline drift was removed using a polynomial subtraction, and the magnitude of the signals was normalized. Activation times across the membrane were determined by the time of maximum change in voltage over time (dV/dtₘₐₓ) of the optical action potentials.

Chronic In Vivo Pacing: The bioresorbable modules in all animals were tested daily post-procedure. Each day, animals were anesthetized with inhaled isoflurane vapors (2-3%) per protocols approved by the Institutional Animal Care and Use Committee (IACUC) at The George Washington University. The Tx coil was placed parallel to the Rx coil of the implanted device to power the bioresorbable module and pace the heart. The frequencies of the pacing stimulation were adjusted such that the stimulation frequency was greater than that of the intrinsic rhythm of the heart. ECG was monitored by subdermal needle electrodes in the Lead I configuration (positive electrode on the right forelimb, negative electrode on the left forelimb, ground electrode on the hindlimb) using a PowerLab data acquisition system with the LabChart software (ADInstruments, Sydney, Australia). The heart rate was calculated from cyclic measurements of the R-R interval on the ECG. Daily testing in the same manner continued until the device failed to capture the ventricular myocardium, as verified by ECG.

Wireless Operation of the Bioresorbable Module: A commercial RF system (Neurolux, Inc., Evanston, IL) was used to wirelessly deliver power to the bioresorbable module for cardiac pacing. The system included the following: (i) a laptop with custom software (Neurolux, Inc., Evanston, IL) to control and command the data center, (ii) a Power Distribution Control box to supply wireless power and communicate with the devices through interactive TTL inputs, (iii) an antenna tuner box to maximize power transfer and match the impedance of the source and the antenna, and (iv) an enclosed cage with customizable loop coil designed for in vivo operation of the devices.

Masson's Trichrome Staining And Immunohistochemistry: Devices were implanted into both male and female Sprague Dawley adult rats. Three groups were analyzed: no implants (control), implants without steroids, and steroid-eluting implants. For all animals implanted with devices, samples were collected at 4 weeks. n=7-12 biologically independent animals per group. To harvest hearts for staining, animals were first deeply anesthetized using 5% isoflurane vapors at 2 mL/min oxygen flow with an EZ anesthesia machine (EZ Systems Inc., EZ-SA 800) until loss of consciousness. Once the cessation of pain was confirmed by toe pinch, the heart was excised for euthanasia by exsanguination. The excised hearts were immediately cannulated and retrograde-perfused first with cardioplegic solution and then with neutral-buffered formalin. After 24 hours, the hearts were transferred to a 70% ethanol solution and embedded in paraffin. Cross-sections underwent either Masson's trichrome staining for assessment of fibrosis or immunohistochemical staining to visualize localization of CD45 in the myocardium.

For Masson's trichrome-stained samples, cross-sections of the anterior left ventricle were imaged at 4× magnification using an EVOS XL light microscope (Thermo Fisher Scientific). A custom MATLAB code was used to quantify the percent volume of myocytes, collagen, and interstitial space in these images. The region of interest was selected along the left ventricular free wall near the site of device implantation. Color deconvolution was performed to identify pink, blue, and white pixels which represent myocytes, collagen, and interstitial space, respectively. The percent volume encompassed by each of these structures was quantified by calculating the relative number of pixels per color in the selected region of interest.

For immunohistochemistry, sections were stained using the peroxidase/avidin-biotin-complex method. Briefly, the procedure was performed as follows: Sections were deparaffinized using xylene and dehydrated using a gradient of concentration of ethanol. Antigen retrieval was performed using citrate buffer (pH=6.0) in a pressure cooker for 20 minutes. Slides were washed 3 times with ultra-pure de-ionized H₂O (3 min/wash). Endogenous peroxidase activity was blocked with BLOXALL solution (Vector Laboratories Cat #SP-6000, RRID:AB_2336257) for 10 minutes. Slides were again washed 3 times with ultra-pure de-ionized H₂O (3 min/wash). Samples were blocked for another 30 minutes with normal goat blocking buffer (TBS, 0.15% Triton X-100, 1% BSA, 3% goat serum [005000121, Jackson ImmunoResearch]). After tapping the blocking buffer off the slides, avidin solution (SP-2001, Vector Laboratories) was applied to the slides for 15 minutes. Slides were then washed for 3 minutes in TBS. The biotin solution (Vector Laboratories Cat #SP-2001, RRID:AB_2336231) was applied to the slides for 15 minutes, which was followed by another 3 minutes wash in TBS. Slides were incubated with the CD45 primary antibody overnight at 4° C. (1:100; Abcam Cat #ab10558, RRID:AB_442810). Next, the samples were washed in TBS with 0.5% Triton X-100 and 2 times in PBS (3 min/wash). Slides were incubated at room temperature with a biotinylated goat anti-rabbit IgG secondary antibody (BP-9100, Vector Laboratories) for 60 minutes and with an avidin biotin complex (ABC) reagent (Vector Laboratories Cat #PK-6100, RRID:AB_2336819) for 30 minutes. Samples were rinsed 3 times in TBS (3 min/wash) before and after application of the ABC reagent. Chromogenic development was achieved using the DAB Peroxidase Substrate Kit (Vector Laboratories Cat #SK-4100, RRID:AB_2336382). Samples were counterstained with Shandon Gill hematoxylin (6765007, Thermo Scientific) for 10 minutes, rinsed with tap water until colorless, and dehydrated in an increasing gradient of ethanol followed by xylene. Slides were mounted with Poly-Mount™ (08381, Polysciences, Inc.), and the images near the site of device implantation were taken using an inverted brightfield microscope (Axioscaope 7, Zeiss) in a tiled manner at 20× magnification using the ZEN Blue software (ZEISS Microscopy). CD45⁺ cells were manually quantified in a blinded manner using ImageJ (v2.1.0, National Institutes of Health). Each counted cell was marked so that no cell was counted twice. A custom MATLAB code quantified the myocardial volume in each image using color deconvolution and calculated the frequency of CD45⁺ cells per mm². To determine statistical differences, a non-parametric Kruskal-Wallis one-way analysis of variance with Dunn's test for pairwise comparison was performed across each condition between the hearts without bioresorbable modules (control), with non-steroid eluting bioresorbable modules, and with steroid eluting bioresorbable modules at a significance level of 0.05.

Echocardiography: Echocardiography was performed on rats before surgery and bi-weekly post-operatively on n=6 biologically independent animals. Rats were anesthetized by inhalation of 2.5% isoflurane vapors at 2 mL/min oxygen flow with an EZ anesthesia machine (EZ Systems Inc., EZ-SA 800). After confirming loss of consciousness, the rat was transferred to the imaging stage. Isoflurane vapors were continuously delivered throughout the imaging session (2.5% at 2 mL/min oxygen flow) so that the heart rate was maintained between 250-300 bpm. Paws were affixed to the stage ECG electrodes with electrode gel and tape to monitor the heart rate throughout the echocardiography session. The chest hair to the left of the sternum was removed so that the ultrasound gel (Aquasonics, clear) could be applied to the skin. M-mode echocardiography of the left ventricle was performed using the Vevo 3100 system (VisualSonics/Fujifilm). The data were analyzed with VevoLAB2.1.0 to measure the following: ejection fraction, stroke volume, cardiac output, fractional shortening, left ventricular end systolic and diastolic diameter, and left ventricular end systolic and diastolic volume. A Friedman test with Dunn's multiple comparison test was performed at a significance level of 0.05.

Enzyme-Linked Immunosorbent Assay (ELISA): Blood samples were drawn from the lateral tail vein of rats implanted with pacemakers 3 weeks after implantation to assess levels of brain natriuretic peptide (BNP 45). n=4 biologically independent animals per group. Blood samples were collected in serum separator tubes (BD, New Jersey, USA), allowed to coagulate for one hour, spun at 2400G for 10 minutes at 4° C. Sandwich enzyme-linked immunosorbent assays (ELISA) were performed with commercially available kits per the manufacturer's protocols in duplicate or triplicate. The following kit was used for BNP 45 (Abcam, ab108816). Briefly, the ELISA is performed as follows: Serum samples and standards were captured by a primary antibody that had been pre-coated onto the bottom of 96-well plates. This primary antibody was then detected by a specific biotinylated detection antibody that was linked with a streptavidin-peroxidase conjugate. The chromogen substrate was catalyzed by streptavidin-peroxidase to visualize the colors in blue. Reactions were stopped, and the absorbance was read at a wavelength of 450 nm. Final concentrations of the biomarkers were determined based on the standard curve. A Mann-Whitney U test with Dunn's multiple comparison was performed at a significance level of 0.05.

Evaluation of Hematology and Blood Chemistry of Rats: All procedures followed protocols approved by Northwestern University Institution Animal Care and Use Committee (IACUC). Blood was collected from adult rats with and without bioresorbable modules implanted using the aforementioned surgical procedures. n=3 animals per group. At 1, 3, 5, and 7-week endpoints, blood was collected from animals via the tail vein into uncoated tubes for blood chemistry tests. A two-way ANOVA with a post hoc Tukey multiple comparison test was performed at a significance level of 0.05.

In Vivo Canine Model Study: Retired breeder female hound dogs (age 1.2-3.5 years, weight 27-36 kg) used in this study were maintained in accordance with the Guide for the Care and Use of Laboratory Animals published by the U.S. National Institutes of Health (NIH Publication No. 85-23, revised 1996) as approved by the IACUC of Northwestern University. Prior to surgery, all animals were premedicated with acepromazine (0.01-0.02 mg/kg) and were induced with propofol (3-7 mg/kg). General anesthesia (inhaled) was achieved with isoflurane (1-3%) after intubation. Adequacy of anesthesia was assessed by toe pinch and palpebral reflex. Surface electrodes were applied to the limbs and continuous 6-lead ECG was recorded at a sampling rate of 977 Hz (Prucka CardioLab). A lateral thoracotomy was performed, and the heart was exposed by pericardiectomy. The electrodes of implanted bioresorbable modules were sutured to the myocardial surface of the right ventricle with 4-0 monofilament non-resorbable sutures. The Rx coil was then placed in a subcutaneous pocket immediately adjacent to the thoracotomy incision. For in vivo long-range tests of wireless operation, the thoracotomy was closed in 4 layers (ribcage, deep fascia and muscles, subcutaneous tissue, and skin). A chest tube was placed prior to closing. The chest was evacuated of air and fluid, and lungs re-expanded. The chest tube was clamped. The selected pacing cycle length was 30-60 ms shorter than the intrinsic ventricular cycle length. Effective ventricular capture was confirmed by surface ECG. Upon finishing the in vivo portion of the study and after confirming a very deep plane of anesthesia, the heart was removed, and euthanasia was achieved by exsanguination.

In Vivo Bioresorption Study: Rats were anesthetized during CT imaging, which was performed, with a preclinical microCT imaging system (nanoScan PET/CT, Mediso-USA, Boston, MA). Data was acquired with a 2.2× magnification, <60 μm focal spot, 1×4 binning, with 720 projection views over a full circle, using 70 kVp/240 μA, with a 300 ms exposure time. The projection data was reconstructed with a voxel size of 68 μm (in all directions) and using filtered (Butterworth filter) back projection software from Mediso. Amira 2020.1 (FEI Co, Hilsboro, OR) was used to segment the device and skeleton, followed by 3D rendering.

Statistical Analysis: Results are reported as mean+SD, unless otherwise noted. Statistical analyses were performed using GraphPad Prism 9. All experiments were performed with at least 3 biological replicates per condition. For quantitative histology and immunohistochemistry analysis, significance in column comparisons was calculated with a non-parametric Kruskal-Wallis test with Dunn's multiple comparison test at a significance level of p<0.05. For echocardiography data, significance in column comparisons was calculated using a Friedman test in conjunction with Dunn's multiple comparison test at a significance level of p<0.05. For ELISA, the significance in column comparisons was calculated using a Mann Whitney U test with Dunn's multiple comparison at a significance level of p<0.05. For hematology and blood chemistry, significance between groups for each factor was calculated using a two-way ANOVA with a post hoc Tukey multiple comparisons test at a significance level of p<0.05.

### Results and Discussion

The System: In the exemplary embodiment, the transient, closed-loop system incorporates a time-synchronized, wireless network with seven key components: (i) a temporary, bioresorbable, stretchable epicardial pacemaker; (ii) a bioresorbable steroid-eluting interface that minimizes local inflammation and fibrosis; (iii) a subcutaneous, bioresorbable power harvesting unit to power the pacemaker; (iv) a set of soft, skin-interfaced sensors that capture electrocardiograms (ECGs), heart rate (HR), respiratory information, physical activity, and cerebral hemodynamics for physiological monitoring of the patient; (v) a wireless radiofrequency (RF) module that transfers power to the harvesting unit; (vi) a soft, skin-interfaced haptic actuator that communicates via mechanical vibrations; and (vii) a handheld device with a software application for real-time visualization, storage, and analysis of data for automated adaptive control. These components integrate into a fully implantable, bioresorbable module 11 having components (i)-(iii), a set of skin-interfaced modules 13 comprising components (iv)-(vi), and an external control module 15 comprising component (vii).

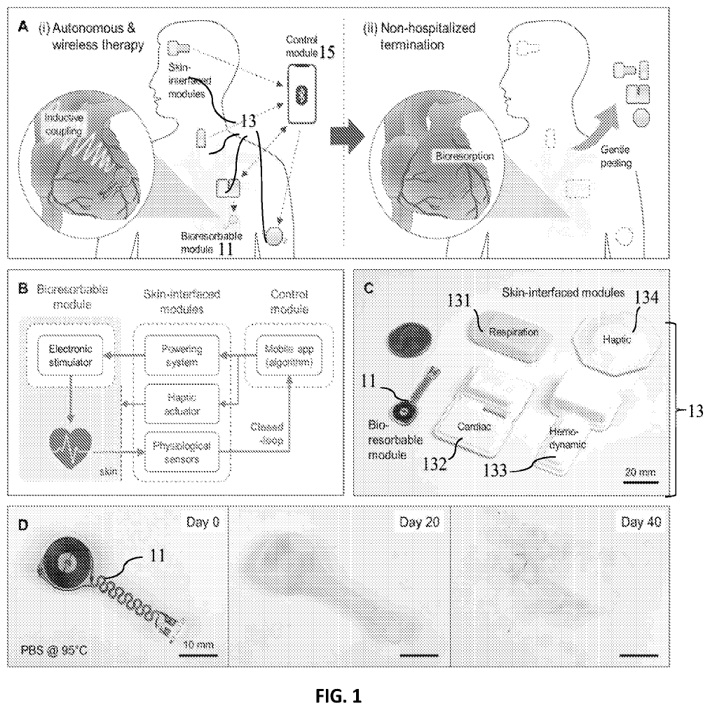

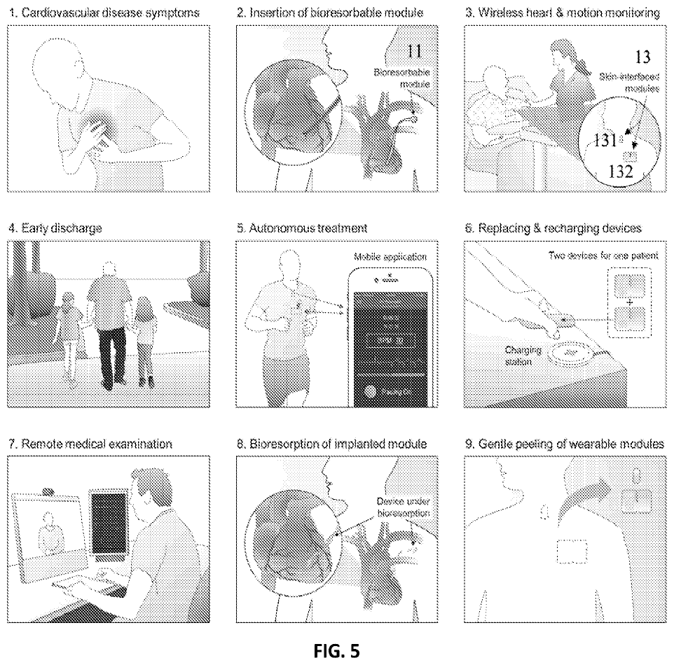

As shown in panel A of FIG. 1, the use of this system is illustrated for temporary cardiac pacing, particularly, (i) autonomous and wireless pacing therapy when modules are positioned for working; and (ii) non-hospitalized termination of the transient closed-loop system. The bioresorbable module 11 wirelessly receives power by inductive coupling to pace the heart through an epicardial interface for epicardial pacing. A network of skin-interfaced modules 13 placed on various locations of the body transmits diverse physiological data to the control module 15 via Bluetooth© low energy (BLE) protocols for real-time data visualization and algorithmic control. A haptic module 134 provides tactile feedback to the patient. After a period of therapy, the bioresorbable module 11 dissolves in the body, and the skin-interfaced modules 13 are removed by peeling them off the skin. These “transient” characteristics of the system eliminate the need for surgical removal and allow ambulatory end of treatment. The clinical use case scenarios are shown in FIG. 5.

Referring to panel B of FIG. 1, a block diagram of the closed-loop scheme that wirelessly interconnects these modules into a wireless network is shown according to one embodiment of the invention. In particular, a diagram of continuous monitoring, autonomous treatment, and haptic feedback based on a wireless, closed-loop system is provided. In one embodiment, the bioresorbable module 11 has an electronic stimulator for cardiac pacing of a patient's heart. The skin-interfaced modules 13 are configured to provide information to and control the bioresorbable module 11; provide power charging to the bioresorbable module 11; collect physiological information from the patient and transmit to the control module; and provide tactile feedback to the patient, and include a powering system, a haptic actuator and physiological sensors. The control module 15 is configured to receive information from the skin-interfaced modules 13, and control the skin-interfaced modules 13. Specifications of the wireless transmission is summarized in Table 1 below.

TABLE 1
Wireless Power/Data Transmission Types For A Transient Closed-Loop System.

| Purpose | Path | Standard | Frequency | Type |
|---|---|---|---|---|
| Heart pacing | Skin-interfaced cardiac | Adjustable | about 13.5 MHz | Inductive |
| | module to implantable | | after body | coupling |
| | module | | loading effect | (Near-field) |
| Wireless | Wireless charger to | NFC | ISM band | Inductive |
| charging | skin-interfaced modules | | (13.56 MHz) | coupling |
| | | | | (Near-field) |
| Data | Skin-interfaced modules | BLE | 2.45 GHz | Radiation |
| transmission | to control module | | | (Far-field) |
| Data | Control module to server | Wi-Fi | 2.4 GHz, 5 | Radiation |
| transmission | | (+Cellular) | GHz | (Far-field) |

Panel C of FIG. 1 shows the soft, flexible designs of the transient system which enables placement of the modules onto various target locations of the body. In particular, the transient system comprises the bioresorbable module 11 and the skin-interfaced modules 13, which, in one embodiment, includes at least one of a respiration module 131, a cardiac module 132, a hemodynamic module 133, and a haptic module 134. In one embodiment, the skin-interfaced modules 13 are attached to the patient without disrupting the integrity of the patient's skin. In one embodiment, the skin-interfaced modules 13 are peeled off from the patient's skin when the system is no longer needed.

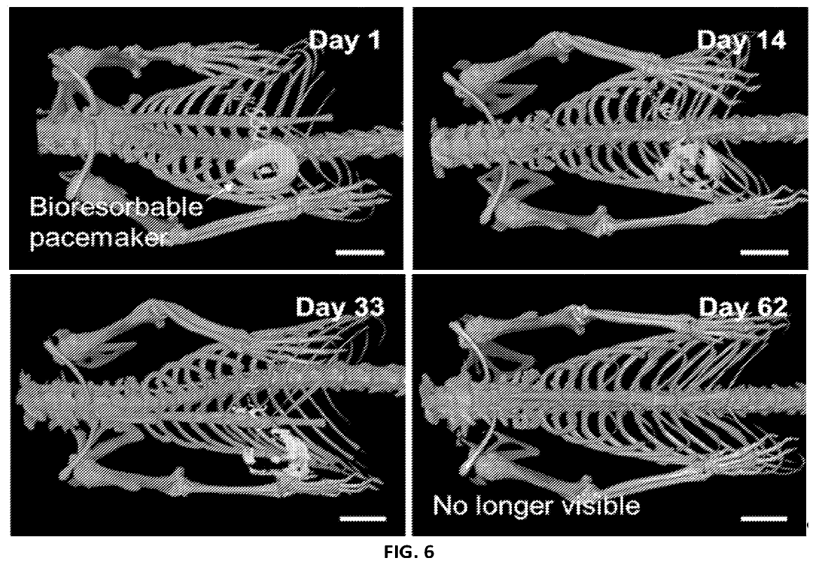

Panel D of FIG. 1 shows that the constituent materials of the bioresorbable module 11 completely disappear in simulated biofluid including of phosphate-buffered saline (PBS). It can be perceived that the constituent materials of the bioresorbable module 11 completely disappear in simulated biofluid including phosphate-buffered saline (PBS). Results of in vivo studies are provided in FIG. 6. In one embodiment, the bioresorbable module 11 dissolves after a period of at least 10 days. In one embodiment, the bioresorbable module 11 dissolves after a period of at least 20 days. In one embodiment, the bioresorbable module 11 dissolves after a period of at least 10 days, 20 days or 30 days. In one embodiment, the period of time before the bioresorbable module 11 dissolves is customizable so as to accommodate the patient's need.

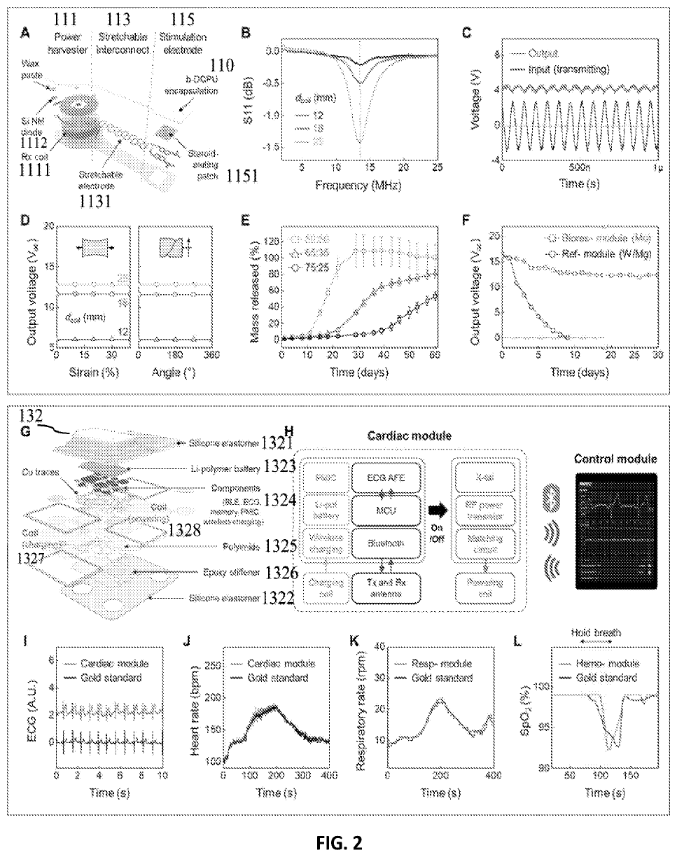

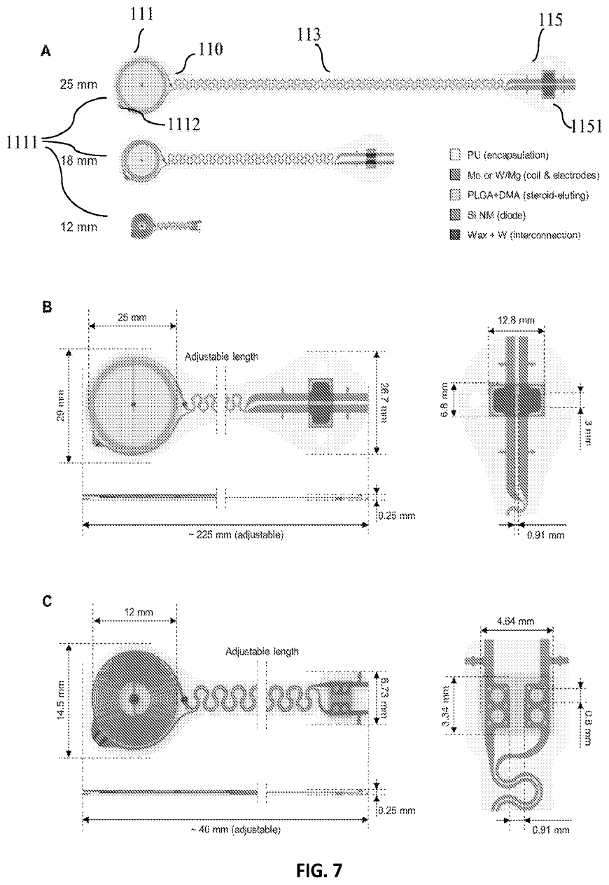

As shown in panel A of FIG. 2, the bioresorbable module 11 includes an RF power harvester 111, which includes an inductive receiver (Rx) coil 1111 (e.g., formed of molybdenum, Mo) and a RF PIN diode 1112 (e.g., formed of silicon nanomembrane, Si NM), a pair of Mo-based stretchable interconnects (Mo) 113, and stimulation electrodes 115 that integrate a steroid eluting patch 1151 at the myocardial interface (pane A of FIG. 2). In one embodiment, the top and bottom encapsulating layers 110 of a bioresorbable dynamic covalent polyurethane (b-DCPU) define a mechanically stretchable structure sealed by thermally activated dynamic bond exchange reactions. The thin, lightweight, and stretchable design of the bioresorbable module 11 minimizes the possibility for irritation or damage at the tissue interface, with geometries that can be tailored to the anatomy of the patient, as shown in FIG. 7.

Pane B of FIG. 2 shows scattering parameters (S₁₁) of the power harvesting unit 111 with three different sizes of the Rx coils 1111. S₁₁ values of the Rx coils with different diameters (black, 12 mm; red, 18 mm; orange, 25 mm) are compared in panel B of FIG. 2. Coils are placed between chicken breast and connected to the load resistance of 1kΩ.

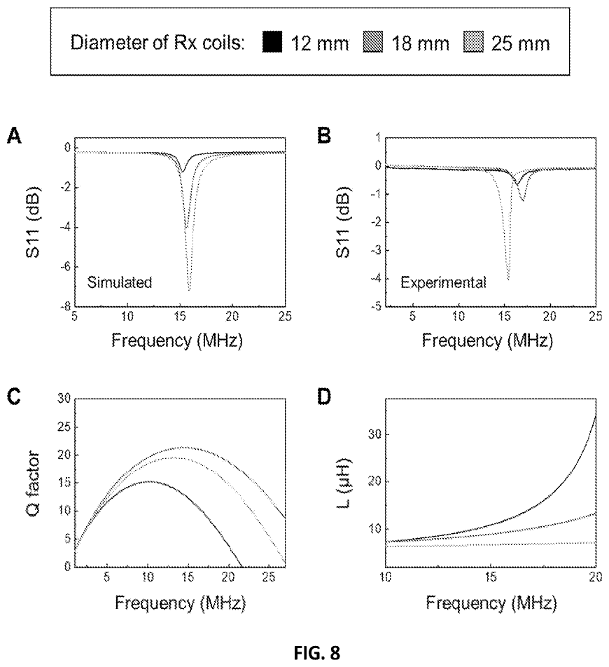

In particular, inductive wireless power transfer in this RF frequency range includes the ISM band (13.56 MHz) which is utilized for battery-free medical implants due to minimal electromagnetic absorption by biofluids or biological tissues. After implantation of the bioresorbable module 11, the resonant frequency shifts due to the electrical permittivity of the surrounding tissue and the tissue-electrode interface. Simulated and experimental results of three coils with different Rx diameters show resonant frequencies of about 15 MHz (FIG. 8) in ambient air in an open circuit configuration. In simulated physiological conditions (device placed between to pieces of chicken breast and connected to 1000Ω load resistance), all three coils exhibit a similar resonant frequency of about 13.5 MHz (panel B of FIG. 2).

The largest coil (25 mm) generates the widest and lowest reflection coefficient

$${S}_{11} = \frac{{Z}_{L} - {Z}_{0}}{{Z}_{L} + {Z}_{0}}$$

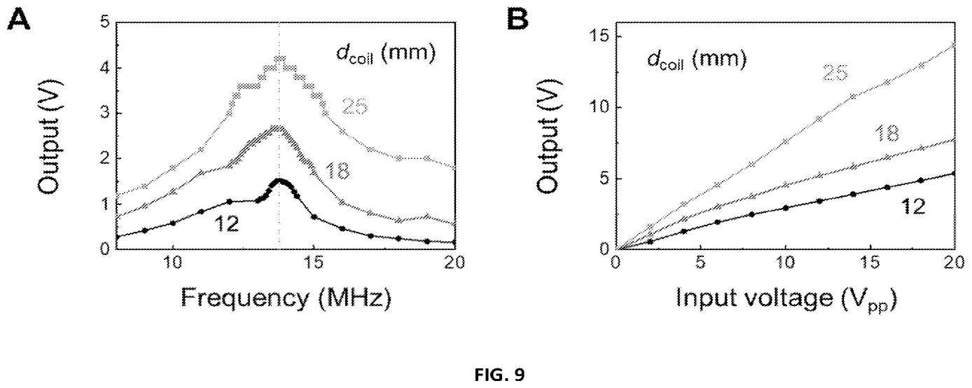

at the resonant frequency, where Z_{L} and Z₀ and the input and source impedance, respectively. Electrical current supplied to a transmission (Tx) circular coil 1328 placed on top of the subcutaneous harvester (Rx coil) 1111 delivers stimulating electrical pulses via resonant magnetic induction, with magnitudes that increase with the coil area and the input driving voltage (FIG. 9). For wireless power transfer, a coupling coefficient k defines the inductive link between the Tx coils 1328 and Rx coils 1111, according to

$$k = \frac{M}{\sqrt{{L}_{Tx} {L}_{Rx}}} ,$$

the ratio between the mutual inductance M and the square root of the product of the individual coil inductances L_{Rx} and L_{Tx}, determined by the relative size and shape of the coils, and distance and angular orientation between them.

As shown in panel C of FIG. 2, continuous alternating current applied to a transmission (Tx) coil 1328 of the cardiac module 132 wirelessly delivers power to the Rx coil 1111 via magnetic induction and induces an approximately direct current monophasic output defined by the diode rectifier. Example output waveform (red; module with 12 mm Rx coil) is wirelessly generated by an alternating current (black; about 3 peak-to-peak voltage (Vpp); transmitting frequency=13.56 MHz) applied to the Tx coil 1328 (12 mm diameter; 3 turns).

With respect to the magnetic resonance imaging (MRI) compatibility of this wireless system, MRI is essential for cardiology patients because of its ability to deliver precise assessment of white matter, gray matter, and posterior fossa abnormalities with functional capabilities that exceed those of ultrasound. The bioresorbable module exploits designs that minimize disturbances in the time-dependent magnetic fields associated with MRI scanning, thereby reducing distortions and shadowing artifacts in the final image and eliminating any parasitic heating from magnetically induced eddy currents. Simulations guide the selection of designs that ensure that the resonant frequencies of the bioresorbable module (about 13.56 MHz) have no overlap with the working frequencies of typical MRI scanners (64 MHz, 128 MHz, 298 MHz, and 400 MHz for 1.5T, 3T, 7T, and 9.4T MRI scanners, respectively), thereby avoiding large gradients of the magnetic field density.

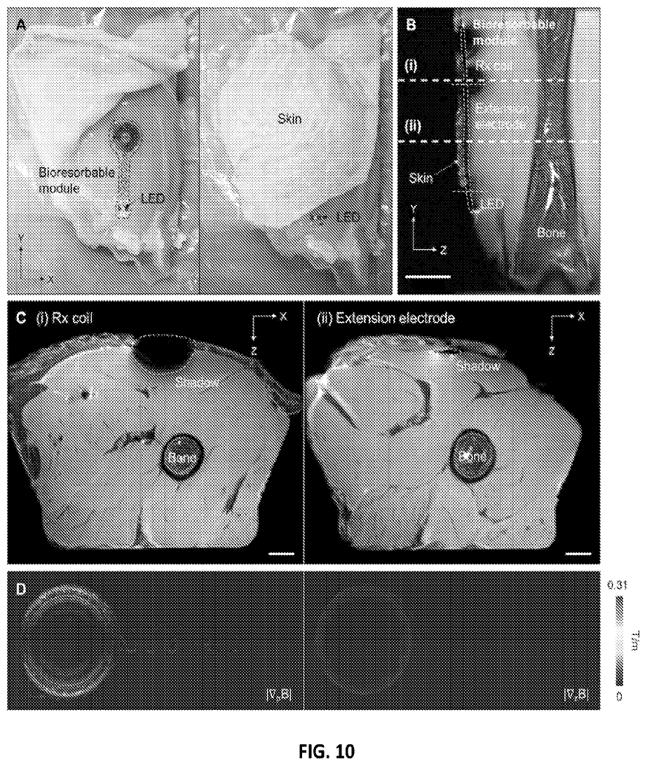

Operation of the transient closed-loop system (e.g., electrical stimulation for heart pacing) in the presence of the MRI scanner (9.4T Bruker Biospec MRI system, Bruker BioSpin Corporation) confirms the ability to operate in an environment with strong magnetic field exposure. As shown in panel A of FIG. 10, the experimental procedure involved placing the bioresorbable module 11 on a chicken thigh subcutaneously and connecting a non-resorbable LED to the exposed electrode. The skin-interfaced cardiac module 13 was placed on the skin of the chicken with good alignment between Tx coil 1328 and Rx coil 1111 to wirelessly power the bioresorbable module 11. Blinking of the LED with a set frequency indicated the proper function of the closed-loop system (e.g., cardiac pacing). Although the Bluetooth-based data transmission was not possible in the MRI room due to magnetic shielding designed to eliminate fringing fields and RF shielding to prevent electromagnetic noise from entering or leaving the room, we observed no changes in the blinking intensity or frequency of the LED even next to the MRI. This uninterrupted operation follows from designs of the skin-interfaced cardiac module that allow it to continuously transfer power to the bioresorbable module even without a Bluetooth connection. The operation was only changed when placed at the entry to and inside the scanner bore, which is surrounded by magnetic gradient coils and RF transmit coils, due to interference with the inductive coupling between bioresorbable module and skin-interfaced cardiac module.

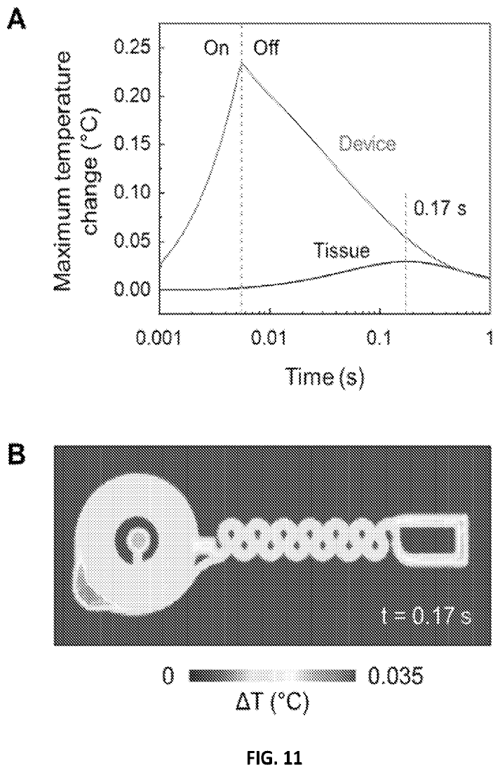

Removing the skin-interfaced module 132 allows us to investigate the MRI compatibility of the bioresorbable module 11 to mimic the situation of a patient who could temporarily function without pacing for the duration of an MRI scan. MR imaging results indicate that the extension electrode causes less susceptibility artifacts (shadowing) compared to the Rx coil 1111 (panels B-C of FIG. 10). In practical scenarios, however, the Rx coil 1111 will be placed subcutaneously, connected to the heart using the extension electrodes 115, thereby minimizing the influence of this part of the device on MR image distortion. Calculations of the gradients of the magnetic field density near bioresorbable modules 11 on biological tissues in a 1.5T MRI scanner reveal the underlying effects. Panel D of FIG. 10 shows that the module induces a weaker disturbance to the magnetic field than commercial electrodes with similar overall sizes and geometries for both the in-plane |∇ₚB| and out-of-plane |∇_{z}B| gradient of the magnetic field density. In this test, the resultant temperature change at the interface with the chicken thigh after MRI scanning is far below the threshold for sensation even in the area near Rx coil 1111 due partly to the insulating effects of the b-DCPU. Full three-dimensional multi-physics modeling also shows that, at the end of a single scan for 1 s, the Mo-based electrodes 115 of the bioresorbable module 11 undergo heating by only 0.23° C. (FIG. 11). The resultant maximum temperature change at the interfaced heart tissue is 0.035° C., far below the threshold for tissue damage or perception of heat (The maximum change in temperature of heart tissue occurs about 0.17 s after initiating the scan). These results meet FDA requirements for an “MRI-safe” label for medical devices and therefore allow the devices to remain in the body during MR imaging.

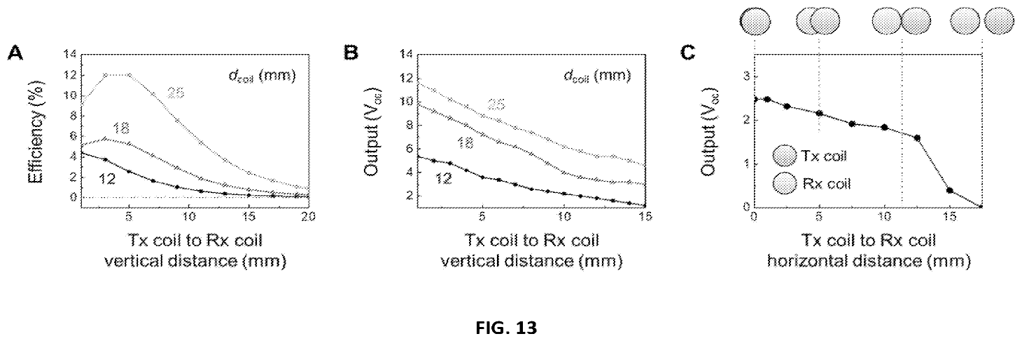

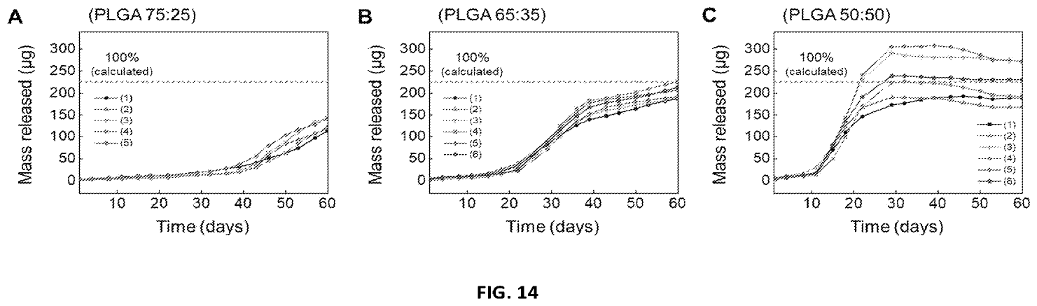

In the invention, optimized mechanical layouts ensure effective and reliable pacing against the mechanically dynamic surface of the heart. Panel D of FIG. 2 shows negligible differences in the output voltage before and after mechanical deformation, consistent with modeling results (FIG. 12). In one embodiment, the output voltage of devices as a function of tensile strain (left) and twist angle (right) at a fixed transmitting voltage (4 Vpp) and frequency (13.56 MHz). Since the wireless energy transfer depends inversely proportional to the coil-to-coil distance (FIG. 13), the Rx unit 1111 resides subcutaneously to maximize the efficiency. Poly(lactic-co-glycolic acid) (PLGA)-based steroid eluting patches 1151 release of dexamethasone acetate (DMA) over the course of several months to minimize local inflammation and fibrosis during cardiac pacing (panel E of FIG. 2 and FIG. 14). Panel E of FIG. 2 shows that the drug release behaviors of steroid eluting patches 1151 with three different ratio (glycolic acid:lactic acid) of base polymer.

The slow rate of dissolution of the bioresorbable conductor (Mo) enables more than one month of functional lifetime under simulated physiological conditions. In particular, panel F of FIG. 2 shows the measurements of output voltages of a bioresorbable module 11 (red square, 10 μm thick Mo) and a reference module (black circle, W/Mg with 700 nm/50 μm thickness) immersed in PBS at physiological temperature (37° C.).

With respect to the life of the bioresorable module 11, the invention discloses at least two different devices with different constituent materials: one uses 10 μm thick Mo electrodes (i.e., bioresorbable module); the other uses tungsten-coated magnesium (W, about 700 nm thick; Mg, about 50 μm thick) electrodes as a control group. The functional lifetime of the W/Mg-based device is only 4 days in the in vivo rat model. To extend this short functional lifetime, the invention (i) introduces a bioresorbable conductor (Mo) with a slow rate of degradation (20 nm/day), (ii) engineers a stretchable electrode structure 1131 by adopting a stretchable polymer encapsulation 110 and exploiting serpentine-structured traces, and (iii) introduces a bioresorbable steroid eluting patch 1151 to reduce the fibrotic tissue growth at the electrode-tissue interface.

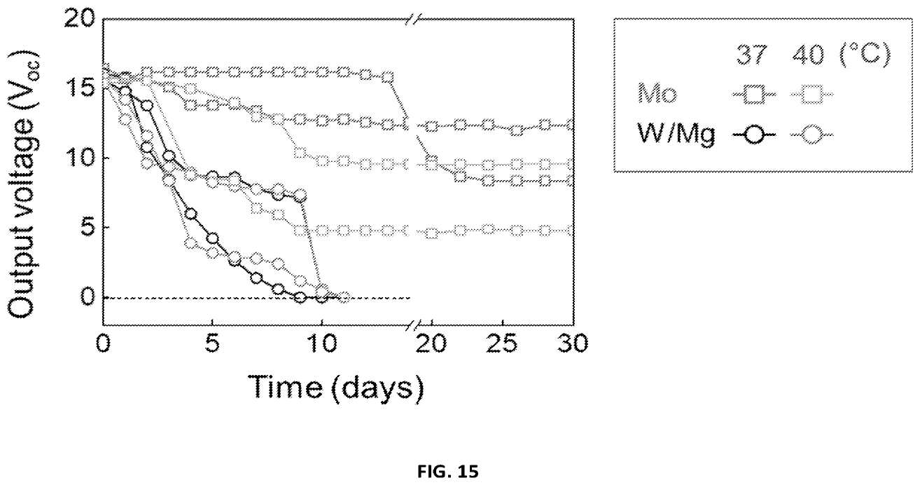

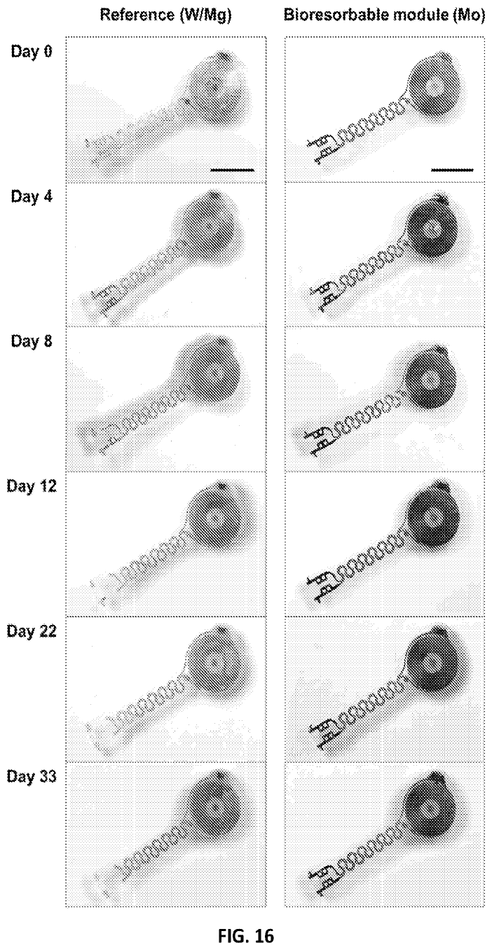

As shown in panel F of FIG. 2, the W/Mg-based reference module (black circle) maintains its function up to 8 days after immersion in PBS (pH 7.4; 37° C.), corresponding to the degradation rate of Mg (about 7 μm/day). The Mo-based bioresorbable module 11 (red square) operates for more than 30 days under the same simulated physiological conditions due to the much slower rate of degradation of Mo (about 20 nm/day). In the physiologically narrow temperature range (37-40° C.), there is no significant differences of the biodegradation behavior (FIG. 15). FIG. 16 shows optical images at various stages of dissolution of bioresorbable modules 11 in PBS (pH 7.4; 37° C.).

The resulting degradation timeline of the single bioresorbable pacemaker (functional lifetime=32 days; full degradation time=500 days) is sufficient for safe administration electrotherapy where temporary pacing is suggested post-operatively, where the required therapeutic window (7 days) of a conventional temporary pacemaker. Within this therapeutic timeframe, it is unlikely that patients will require an additional pacemaker.

The degradation (i.e., resorption) time of the bioresorbable module may vary across patients due to intrinsic physiological differences, such as the amount of biofluid and enzymes, the rate of the recovery, level of the activity, and speed of metabolism. The functional lifetime of the bioresorbable module can be tailored by selecting the appropriate thicknesses and types of constituent materials to meet the needs of the patient and their required period for the therapeutic treatment.

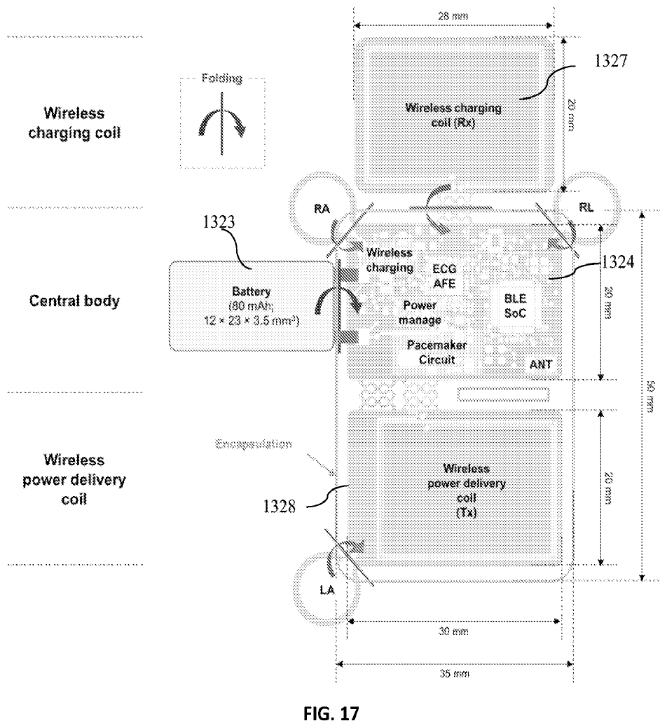

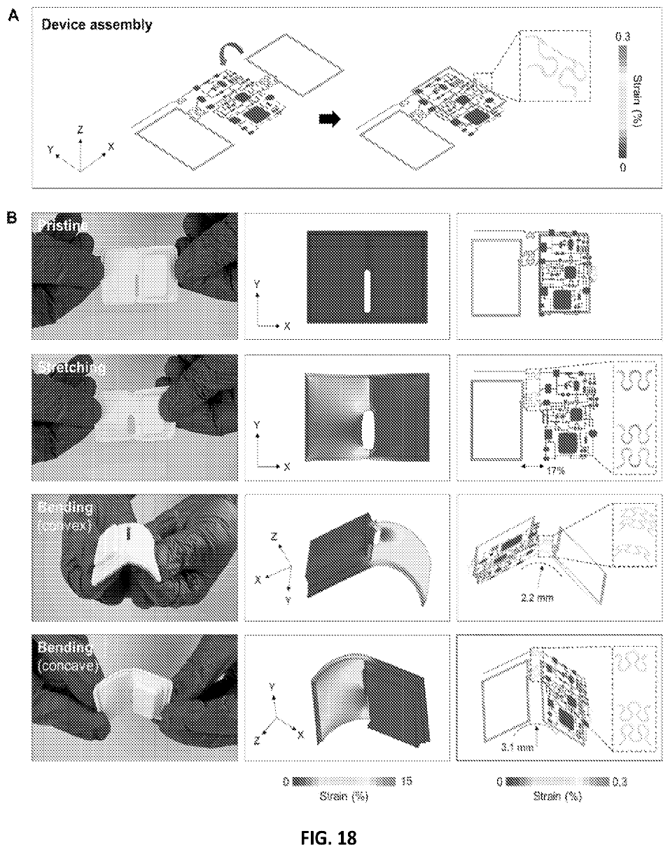

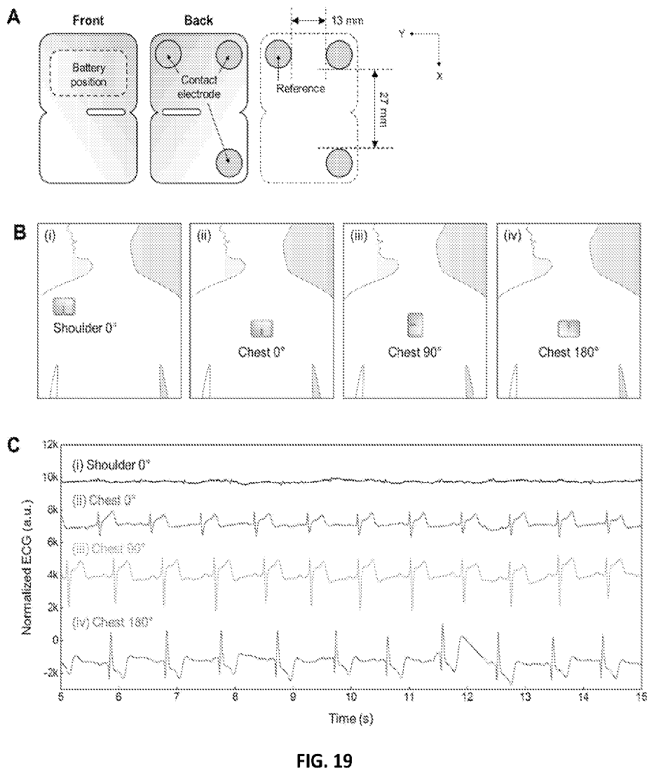

A network of the skin-interfaced modules 13 placed on various locations of the body acquires diverse data relevant to patient status. These collective data streams form the basis for closed-loop control. The cardiac module 132 is the most essential component, and is placed on the chest to collect physiological information and to provide RF power to the bioresorbable module 11. As shown in panel G of FIG. 2 and FIG. 17, the materials and architectures follow design principles of soft electronics to ensure robust, irritation-free coupling to the skin (FIG. 18) at relevant locations using hydrogel adhesives (FIG. 19). In one embodiment, panel G of FIG. 2 shows the exploded-view schematic illustration of the skin-interfaced cardiac module 132. In one embodiment, the skin-interfaced cardiac module 132 comprises an upper silicone elastomer 1321 and a bottom silicone elastomer 1322, a Li-polymer battery 1323, components 1325 including BLE, ECG memory, PMIC, wireless charging elements, polyimide 1325, epoxy stiffener 1326, wireless charging coil 1327 and wireless powering delivery coil 1328.

The multi-haptic module 134 on the mid-medial forearm and dorsal interosseous provides information on patient status and device operation through up to 625 patterns of vibro-tactile input. The respiratory module 131 mounts at the suprasternal notch to capture physical activity, body temperature and respiratory behavior, in a dual-sensing design for accurate operation. The hemodynamic module 133 on the forehead in one embodiment uses a pair of light emitting diodes (LEDs) and four photodetectors (PDs) to measure peripheral blood oxygen saturation (SpO₂) at depths from the scalp to the brain.

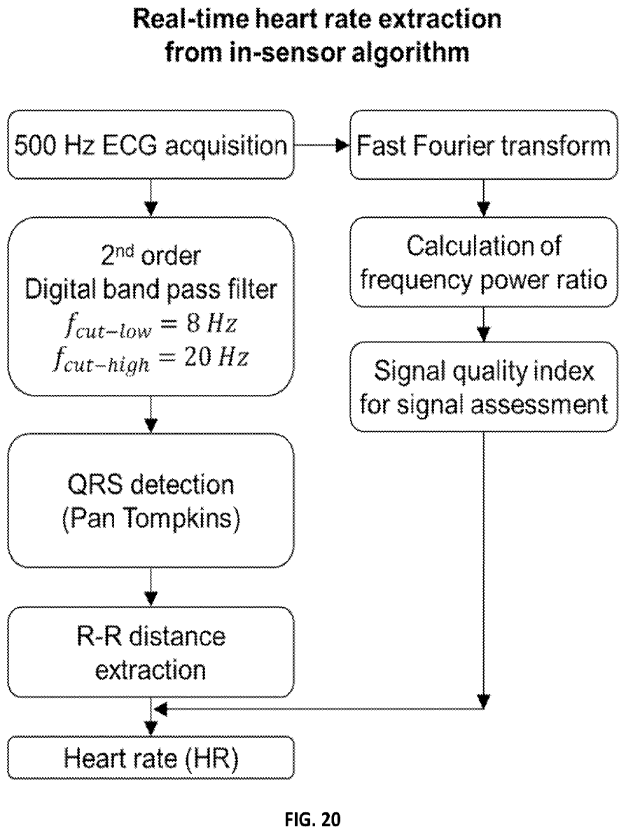

Panel H of FIG. 2 is a block diagram of the skin-interfaced cardiac module. In particular, it is a system block diagram of the operation of the cardiac module. PMIC indicates power management integrated circuit. An ECG analog front end and (AFE) and a microcontroller unit (MCU) in the cardiac module process measured data in real-time to calculate the heart rate, for example, using the Pan-Tompkins algorithm (FIG. 20).

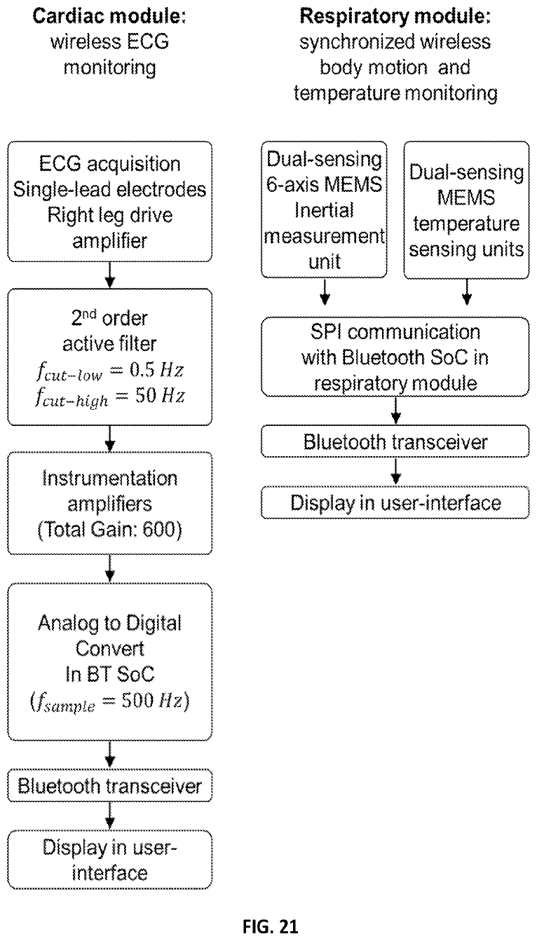

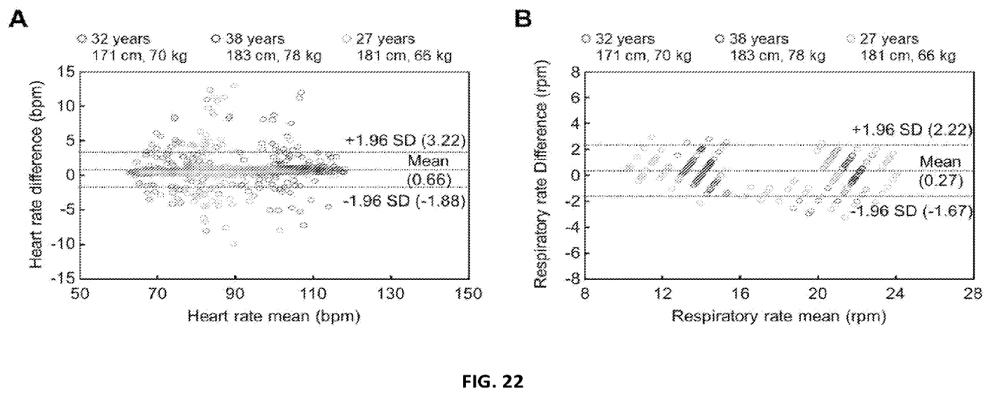

A BLE-enabled user-interface serves as a control unit that stores and displays ECG tracings and 3-axis acceleration data associated with cardiac and respiratory activity (FIG. 21 shows the signal processing block diagram). Panels I-K of FIG. 2 shows that the skin-interfaced modules 13 and data analytics approaches accurately determine heart rate and the respiratory rate (FIG. 22). In particular, panel I of FIG. 2 provides representative ECG results collected from the cardiac module 132 (red) and a reference device (black), while panels J-L of FIG. 2 reflect comparisons of heart rate, respiratory rate, and SpO₂ level determined by the skin-interfaced modules 13 (red; J, cardiac 132; K, respiratory 131, L, hemodynamic 133) and a reference device (black). In panel L of FIG. 2, healthy subjects hold their breath for 60 s (yellow background).

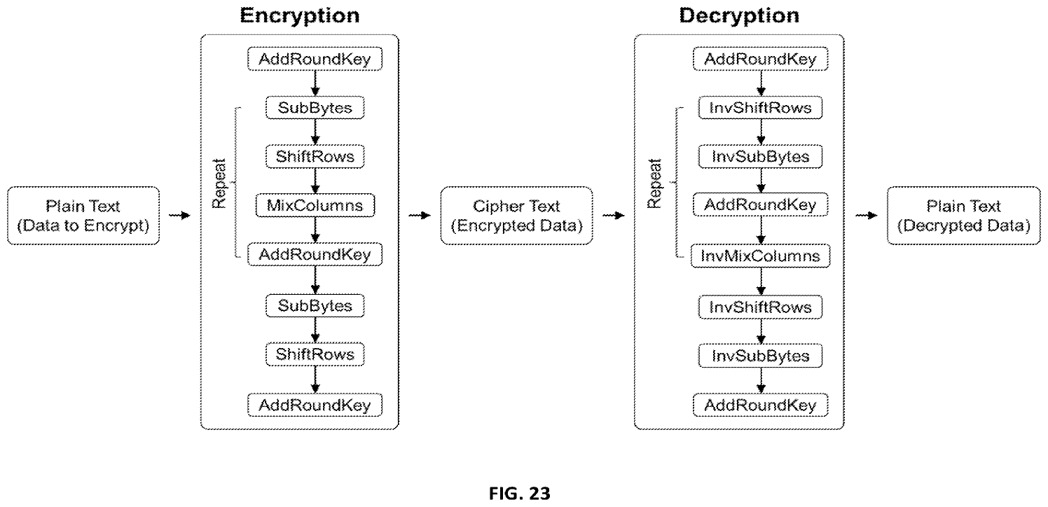

The hemodynamic module 133 yields SpO₂ data comparable to data recorded by a medical-grade finger probe (panel L of FIG. 2). These systems exploit current best practices to protect health data, from the sensor, the Bluetooth link, the phone, the cloud, and beyond. To ensure secure medical data storage and processing, the interface application is compatible with hypertext transfer protocol secure (HTTPS) transport layer security (TLS 1.2) and with algorithms for encryption/decryption (FIG. 23). In-sensor encryption (advanced encryption standard (AES)-128) and Health Insurance Portability and Accountability Act (HIPAA)-compliant cloud data storage further protect patient data.

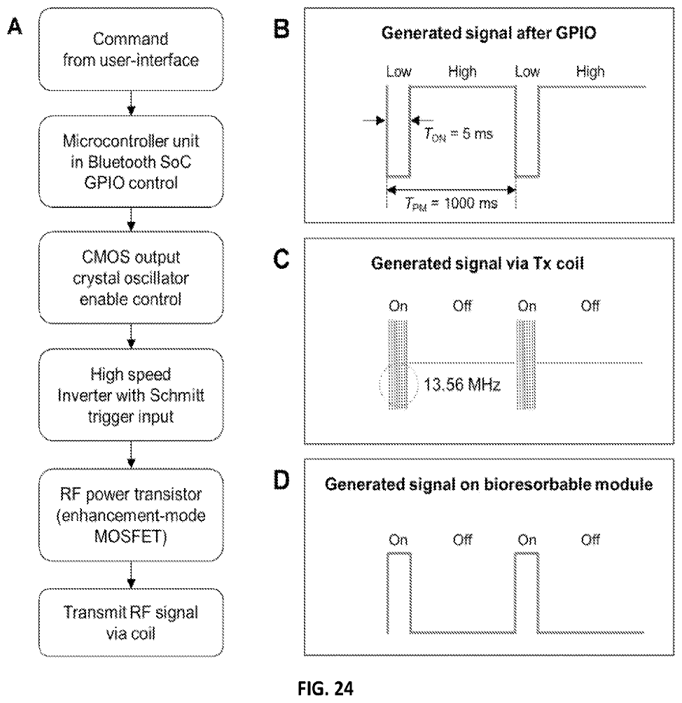

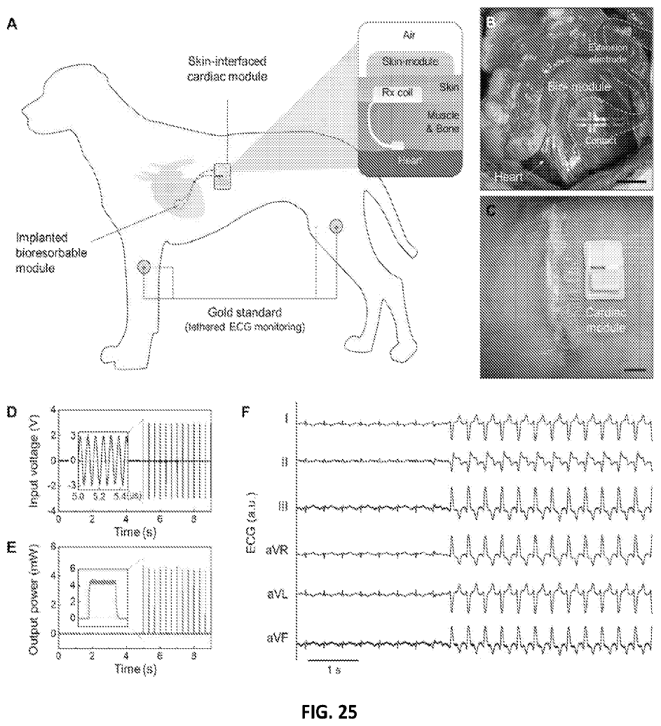

According to the invention, one of the key features of the transient closed-loop system is that the skin-interfaced cardiac module 132 eliminates requirements for wall-plugged, external hardware for power transfer and control of the implanted pacemaker (FIG. 24). In vivo studies with a canine whole-heart model demonstrate the capabilities (FIG. 25). When the wireless cardiac module 132 generates pulsed alternating currents (6 peak-to-peak voltage Vpp), the power harvester coil 111 in the bioresorbable module 11 rectifies the received waveform to a pulsed direct current output (about 4 mW) and delivers it to the interface with the myocardium as a cathodic monophasic pulse through the electrode pads. Investigations using rodent models demonstrate continuous, long-term pacing and bio-compatibility.

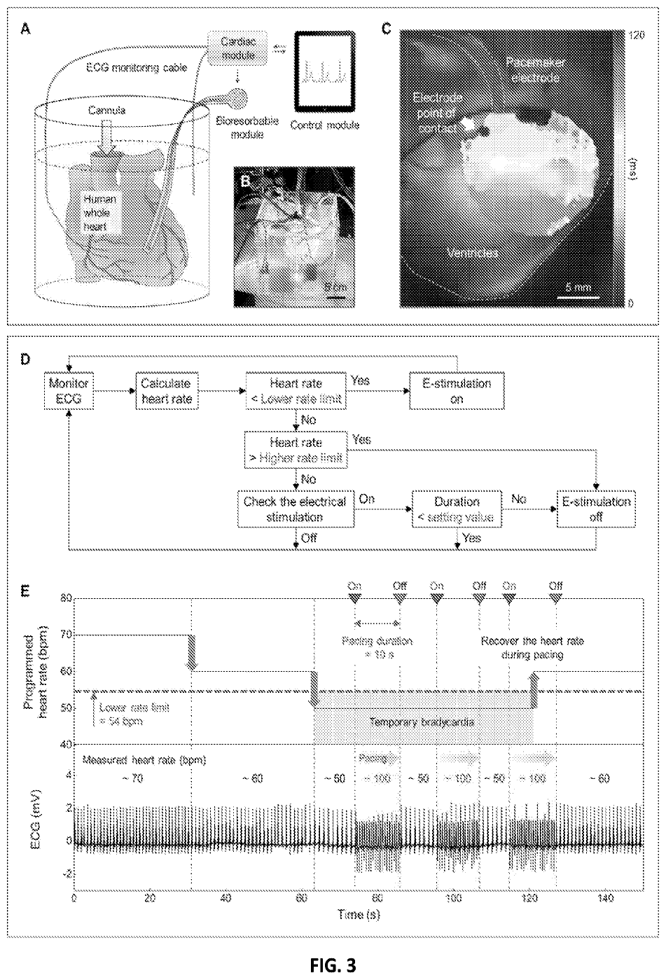

Autonomous Treatment Function: An additional capability of the transient closed-loop system is in autonomous treatment based on algorithmic identification of ECG signatures of abnormal cardiac activity. For example, hysteresis pacing delivers programmed electrical stimuli if the intrinsic rate falls below a certain threshold, to avoid overriding slow but appropriate intrinsic rhythms. As shown in FIG. 3, ex vivo human whole heart studies demonstrate this type of treatment for the case of temporary bradycardia.

Panel A of FIG. 3 is a schematic illustration of a Langendorff-perfused human whole heart model of bradycardia with a transient closed-loop system. A cardiac module is connected with ECG monitoring cable. Panel B of FIG. 3 is a photograph of the setup (Rx coil diameter is 25 mm).

Anisotropic activation of the membrane potential confirms that the bioresorbable module 11 is the driving source of cardiac activation, according to panel C of FIG. 3. Activation map of the human epicardium near the pacing electrode, showing that activation originates from the location of the electrode pad of the device (arrow). Panel D of FIG. 3 shows a flow chart of the feedback control system implemented in the mobile application describing the hysteresis pacing scheme by which the system recognizes bradycardia and activates pacing during the programmed period of treatment. In particular, the flow chart of closed-loop hysteresis pacing to activate the pacemaker upon automatic detection of bradycardia. The sensors in the cardiac module 132 monitor cardiac electrocardiograms. Real-time signal processing methods classify the bradycardia based on a heart rate value that falls below a threshold value (=lower rate limit). In this condition, the system automatically activates the bioresorbable module 11 wirelessly, to provide electrical stimulation to the myocardium at a pre-specified rate. If the heart rate is above a certain threshold (=higher rate limit), the system remains inactive. This feedback control algorithm maintains the heart rate within a programmed range without arrhythmia. The pacing can also be automatically terminated after a programmed period (=pacing duration), allowing clinical operators to monitor and reassess the underlying heart rhythm of the patient between pacing treatments. These functions (lower and higher rate limit, and pacing duration), along with the various parameters associated with the pacing waveforms (pacing frequency, pulse width) can be defined manually on the user interface 15 to address specific patient needs. In clinical practice, the pacing inhibition function is limited to brief, pre-specified times when the patient is supine to minimize risks associated with interruption of pacing.

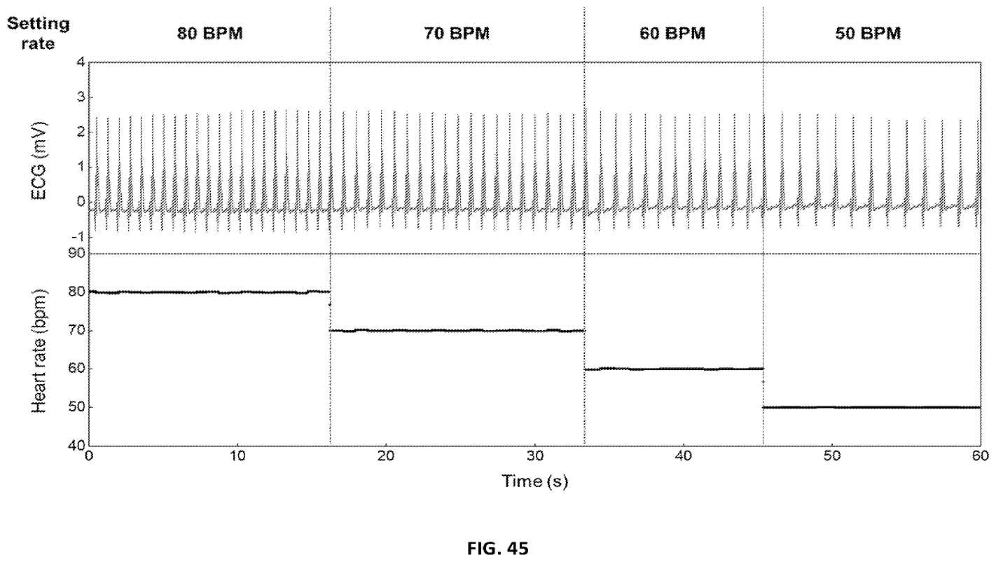

A separate pacing electrode enables manual control of the heart rate to mimic bradycardia (FIG. 45). Panel E of FIG. 3 shows that the transient closed-loop system detects bradycardia (less than 54 bpm, in this case, the bradycardic threshold is set to 54 beats per minute (bpm)) and automatically initiates pacing (about 100 bpm). After a predetermined pacing duration (10 s), the system automatically stops pacing and evaluates the underlying intrinsic ECG signals to determine the need for additional pacing treatment. When the heart recovers from temporary bradycardia, the system detects the normal physiological rhythm (about 60 bpm) and ceases to deliver on-demand pacing. The top section in panel E of FIG. 3 is programmed heart rate, and the bottom section in panel F of FIG. 3 is ECG of a human whole heart. Set parameters are lower rate limit, 54 bpm; pacing duration, 10 s; pacing rate, 100 bpm. For demonstration purposes, setting the pacing rate to a value much higher than the lower rate limit clearly distinguishes intrinsic and paced rhythms. In clinical practice, this pacing rate would be closer to the lower rate limit.

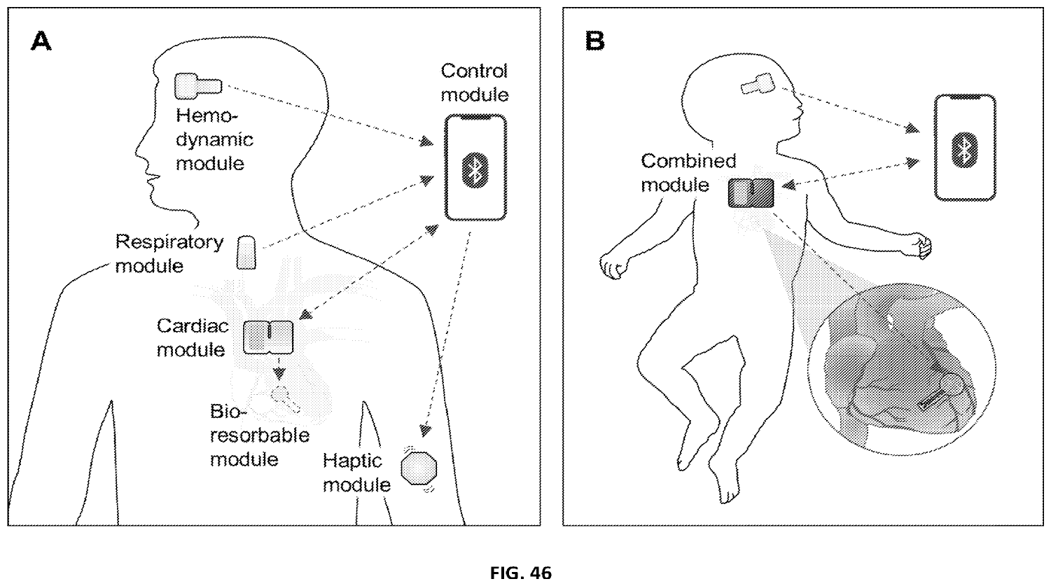

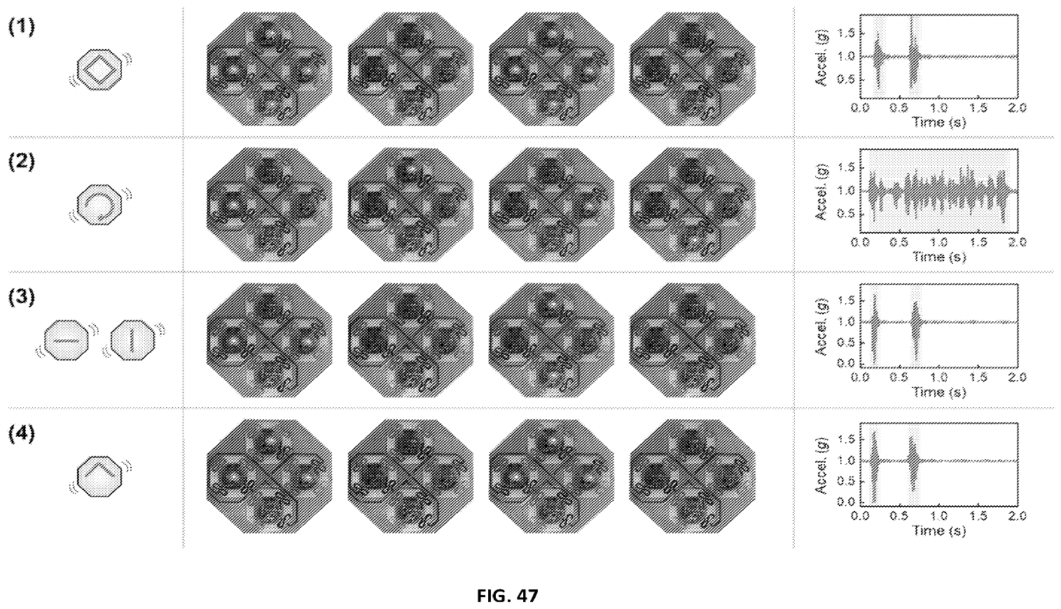

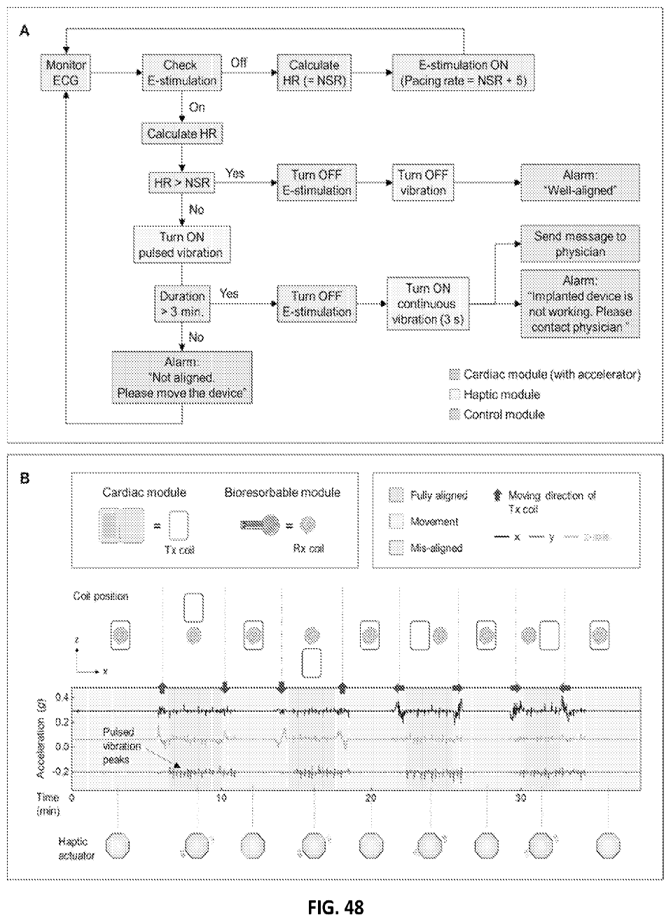

For advanced forms of operation, the control module 15 wirelessly communicates with the full collection of skin-interfaced modules 13 via BLE protocols, in a manner that is expandable and customizable to accommodate wide-ranging types of devices with various actuation, feedback and/or monitoring capabilities. The schematic illustration in panel A of FIG. 4 and FIG. 46 summarizes the most sophisticated system configuration of the invention. In particular, panel A of FIG. 4 shows a schematic illustration of a transient closed-loop system of wireless, body-integrated devices for physiological monitoring, automated control, and patient feedback. This network of modules also includes the option to deliver tactile inputs through different patterns of vibration (FIG. 47) to inform the patient of (i) the remaining battery life of the cardiac module 132 (panel B of FIG. 4, i); (ii) its proper operation of the cardiac module (panel B of FIG. 4, ii); (iii) instances of malfunction of any of the modules (panel B of FIG. 4, iii), and (iv) symptoms of bradycardia (panel B of FIG. 4, iv). Panel B of FIG. 4 demonstrates the patient awareness function using the multi-haptic module. Accelerometer data (z-axis) corresponds to the vibration of the haptic actuators. The haptic module 134 can also be activated to facilitate positioning and alignment of the cardiac module 132 to bioresorbable module 11 during mounting, of particular importance in the course of device replacement for recharging (FIG. 48).

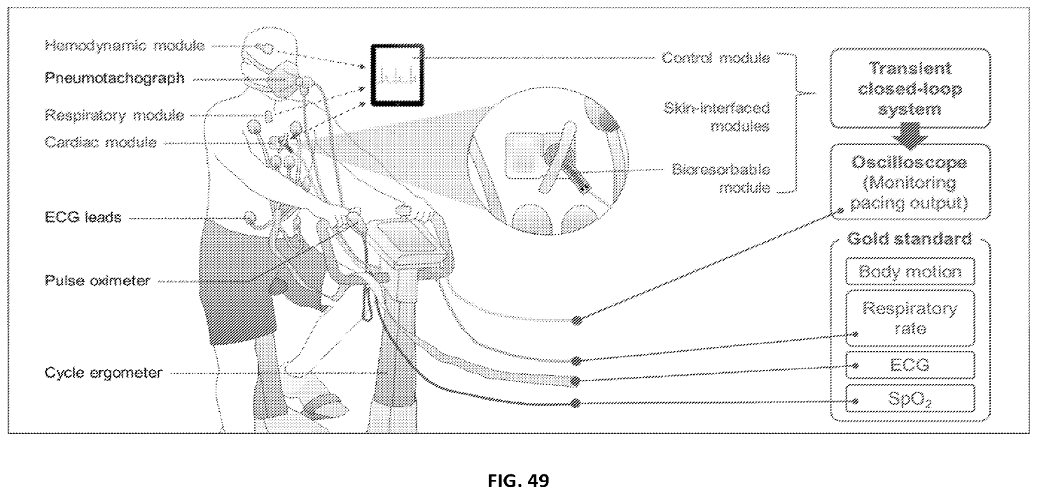

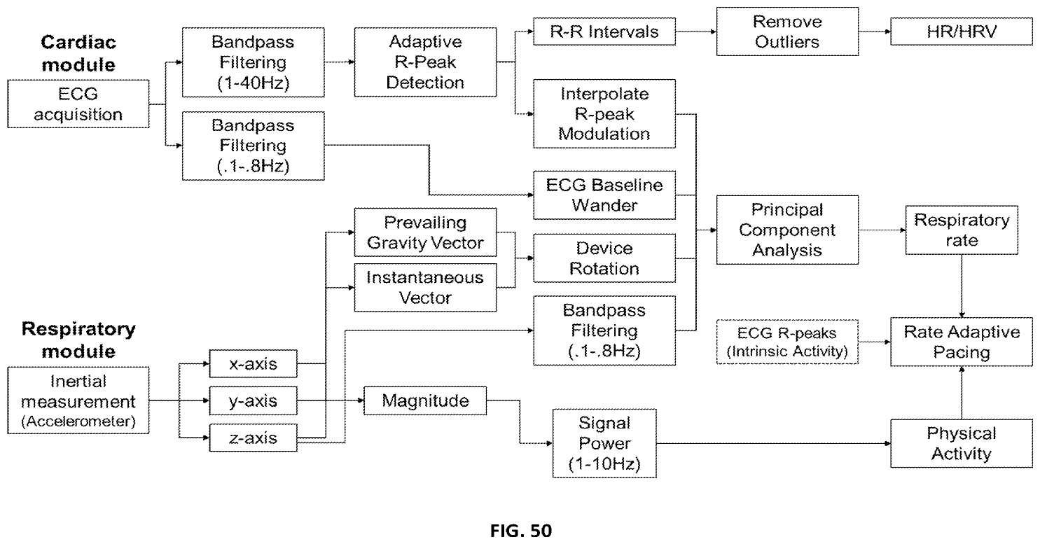

Real-time monitoring of cardiopulmonary status via comprehensive, multi-modal measurements enables elaborate schemes in rate-adaptive pacing. Certain conventional pacemakers use accelerometers to determine metabolic needs via measurements of motion of the thorax, but this method requires custom programming and threshold adjustment by a trained clinician. More importantly, the utility is limited when increased metabolic demand does not correlate with motion of the core body, such as with stationary exercise. The wireless network reported here captures both body motions and respiratory activity, along with other essential parameters in a scalable fashion, to enable closed-loop algorithms with high levels of robustness and adaptability. Schematic illustrations in FIG. 49 describe the experimental setup for exercise tests with healthy human subjects on stationary bicycles. Physical activity can be estimated from the magnitude of the triaxial accelerometer data using a developed algorithm (FIG. 50).

Panel C of FIG. 4 shows the results of clinical tests with a healthy human subject. Data were recorded simultaneously over 8 min as the subject rests and engages in slow and fast cycling: (i) calculated physical activity using data from the respiratory module 131. (ii) Calculated respiratory rate using data from the respiratory module 131. (iii) Comparison of the heart rate (black) of a healthy human subject monitored by the cardiac module 132 and rate-adaptive pacing signals (red) processed from the transient closed-loop system. (iv) Calibrated and measured changes in core body temperatures using data from the respiratory module 131. (v) Representative oxygen saturation level measurements from the hemodynamic module 133.

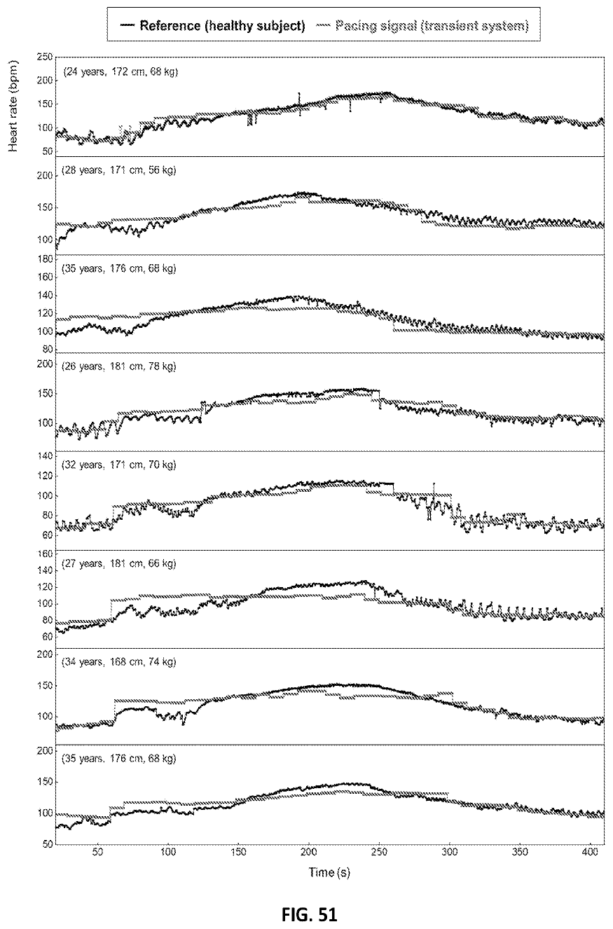

In one embodiment, panel C of FIG. 4, section (i) shows a strong qualitative correspondence between measured physical activity and exercise intensity (e.g., rest, slow, fast) and measured physical activity. The respiratory rate shows a time-delayed correlation to physical activity and has gradual changes at the transition of exercise intensities, as shown in section (ii) in panel C of FIG. 4. Panel C of FIG. 4, section (iii) indicates that the pacing rate dynamically adjusts to the respiratory rate and physical activity level, determined during a pre-defined window (in this case, every 10 s). For this healthy subject, the heart rate properly reflects that the metabolic demand is consistent with the level of physical activity and the respiratory rate determined by the respiratory module 131. The pacing signal (iii), calculated by (i) and (ii), shows good agreement with the HR of the healthy subject because the metabolic demand is consistent with the level of exercise intensity and respiration. Results from different human subjects (n=8) confirm the reliability of this algorithm. Results from different human subjects (n=8) confirm the reliability of this algorithm (FIG. 51).

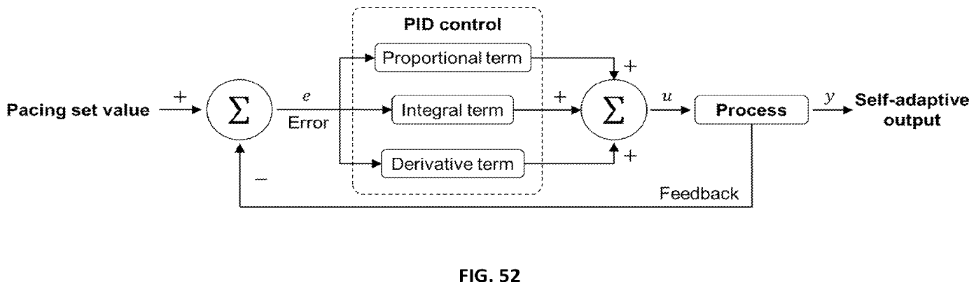

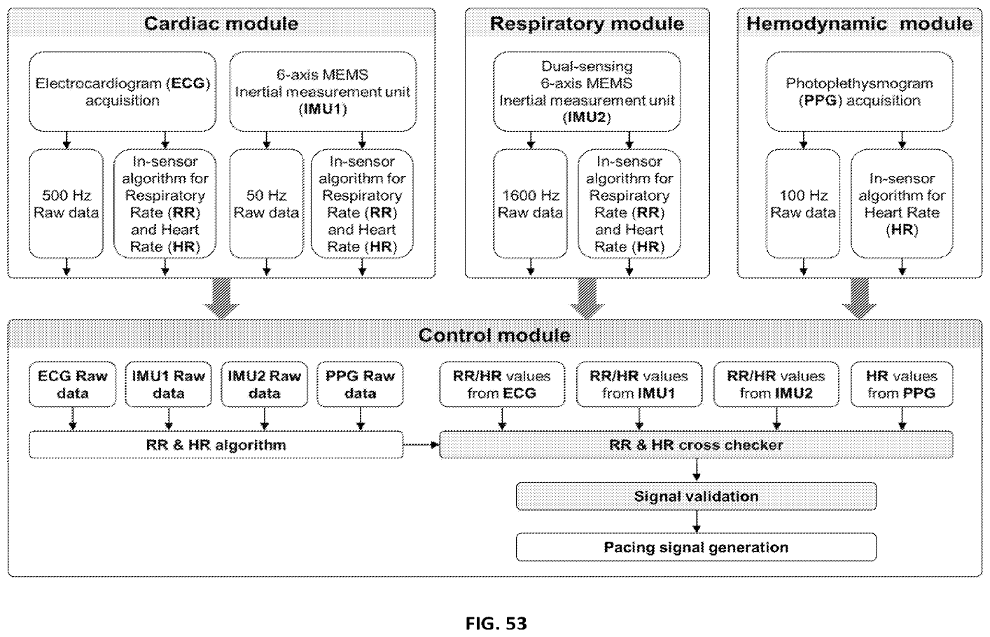

Using respiratory rate in addition to accelerometer measurements also limits inappropriate increases in pacing rate that results from external motion. For example, the sensor may detect an apparent increase in physical activity from an external source (e.g., tapping on the sensor or a bumpy car ride) with no increase in respiration rate. In these aberrant cases, the system maintains the pacing rate below a certain level (e.g., 60 or 70 bpm) since the respiratory rate does not rise significantly above resting levels (e.g., 20 rpm). In addition, Proportional-Integral-Derivative (PID) control yields a more stable and reliable pacing rate by using the error between the pacing set value and the measured value continuously against disturbances including sudden electric shocks due to large RF pulses or defibrillation processes (FIG. 52). The monitoring physician can also substitute algorithms currently used in state-of-the-art permanent pacemakers into this hardware system and make setting adjustments remotely. A cross-check validation method ensures signal quality. FIG. 53 shows the block diagram of the cross-check validation scheme for generating reliable pacing signals. The control module processes real-time HR and respiratory rate locally and performs cross-checking validation with the transmitted HR and respiratory rate values from the networked collection of skin-interfaced devices including cardiac, respiratory, and hemodynamic modules. This method prevents misguided pacing signal generation protecting against device malfunction and external hacking or spoofing.

Other physiological parameters, such as body temperature (section (iv) in panel C of FIG. 4) and blood oxygen saturation level (section (v) in panel C of FIG. 4), provide additional information that is postoperatively useful for patients with limited cardiopulmonary reserve, slowly resolving pneumonia, or persistent supplemental oxygen requirements.

Briefly, the transient, closed-loop system disclosed in the invention represents a distributed, wireless bioelectronics technology that provides autonomous electro-therapy over a time frame that matches postoperative needs. The operation involves coordinated operation of a network of skin-interfaced modules and a bioresorbable device in time-synchronized communication with a control platform. Data captured from various locations of the body yield detailed information on cardiopulmonary health and physical activity. The results define autonomous, rate-adaptive pacing parameters to match metabolic demand through wireless powering of the bioresorbable module; they also support feedback on device and physiological status through a multihaptic interface. The bioresorbable module for cardiac pacing undergoes complete dissolution by natural biological processes after a defined operating time frame. The skin-interfaced devices can be easily removed after patient recovery. This system provides a framework for closed-loop technologies to treat various diseases and temporary patient conditions in a way that can complement traditional biomedical devices and pharmacological approaches.

### Example 2

### Clinical Use Case Scenarios

Arrhythmias are common complications after cardiac surgery and represent a major source of morbidity and mortality. Bradyarrhythmias are particularly common after valve surgery and are a consequence of direct surgical injury and local edema. After valve surgery and/or coronary artery bypass graft (CABG) surgery, bradycardia usually is caused by sinus node dysfunction or atrioventricular conduction disturbances. In these cases, temporary pacemakers are implanted to treat the transient arrhythmias. Temporary pacemakers may also act as a bridge to therapy, because a permanent pacemaker is implanted if symptomatic bradyarrhythmias, such as atrioventricular block and sick sinus syndrome, persist longer than 5-7 days postoperatively. If the underlying intrinsic rhythm is absent or temporary pacing leads fail, permanent pacing may be performed earlier.

The transient closed-loop system and its intelligent pacing algorithm are designed to address all bradycardias to increase the heart rate when it is too low, and it does not provide termination of peroxisomal or sustained mono- or polymorphic tachyarrhythmias. Future work includes development anti-tachycardic pacing and multi-pulse therapy protocols in order to terminate ventricular tachycardias with this system without the need for high voltage shocks.

FIG. 5 illustrates the envisioned use case scenarios of the transient closed-loop system, which include, but are not limited to, the following exemplary cases. Case scenario 1 shows a patient presents symptoms indicating need for electronic pacemaker implantation. Case scenario 2 shows a bioresorbable module 11 is implanted with electrodes attached to the heart muscle. The receiver (Rx) coil 1111 of the device is placed in a subcutaneous pocket. Case scenario 3 shows that, throughout the treatment period, the skin-interfaced cardiac 132 and respiratory modules 131 are placed on the chest and suprasternal notch, respectively. The other skin-interfaced devices 13, such as hemodynamic 133 and haptic modules 134, can also be included. The patient's mobility and daily activities are only minimally impeded due to the wireless nature of the system. Case scenario 4 shows that the above mentioned and other features of the system enable the patient to complete inpatient treatment quickly for early hospital discharge. Case scenario 5 shows the transient closed-loop system provides continuous monitoring and autonomous treatment through coordinated operation of (i) an implanted bioresorbable module 11, (ii) a collection of skin-interfaced modules 13, and (iii) an external control module 15. For example, if the external control module detects bradycardia from the real-time ECG data, the skin-interfaced cardiac module delivers power and control signals to the implanted bioresorbable module via inductive wireless energy transfer to pace the heart. Case scenario 6 shows two sets of each skin-interfaced module 13 and one tethered stimulator can be provided to allow continuous electrical stimulation. The battery in the skin-interfaced modules 13 can be recharged wirelessly. Case scenario 7 shows wireless and automatic transfer of diagnostic health information to a clinician enables remote medical care. This platform may allow clinicians to detect changes early and to make proactive decisions, to improve patient outcomes. Case scenario 8 shows following resolution of pacing needs or insertion of a permanent device, the implanted device dissolves into the body, thereby eliminating the need for surgical extraction. Case scenario 9 shows the skin-interfaced modules 13 can be physically removed by gentle peeling from the skin, terminating the medical treatment without the need to return to the hospital.

### Example 3

### Electromagnetic Characteristics of Bioresorbable Module

Inductive wireless power transfer in the RF frequency range including the ISM band (13.56 MHz) is utilized for battery-free medical implants due to minimal electromagnetic absorption by biofluids or biological tissues.

After implantation of the bioresorbable module, the resonant frequency shifts due to the electrical permittivity of the surrounding tissue and the tissue-electrode interface. Simulated and experimental results of three coils with different Rx diameters show resonant frequencies of about 15 MHz (FIG. 8) in ambient air in an open circuit configuration. In simulated physiological conditions (device placed between to pieces of chicken breast and connected to 1000Ω load resistance), all three coils exhibit a similar resonant frequency of about 13.5 MHz (panel B of FIG. 2).

The largest coil (25 mm) generates the widest and lowest reflection coefficient

$${S}_{11} = \frac{{Z}_{L} - {Z}_{0}}{{Z}_{L} + {Z}_{0}}$$

at the resonant frequency, where Z_{L} and Z₀ and the input and source impedance, respectively. Electrical current supplied to a transmission (Tx) circular coil placed on top of the subcutaneous harvester (Rx coil) delivers stimulating electrical pulses via resonant magnetic induction, with magnitudes that increase with the coil area and the input driving voltage (FIG. 9). For wireless power transfer, a coupling coefficient k defines the inductive link between the Tx and Rx coils, according to

$$k = \frac{M}{\sqrt{{L}_{Tx} {L}_{Rx}}} ,$$

the ratio between the mutual inductance M and the square root of the product of the individual coil inductances L_{Rx} and L_{Tx}, determined by the relative size and shape of the coils, and distance and angular orientation between them.

### Example 4

### MRI Compatibility of the Bioresorbable Module

Magnetic resonance imaging (MRI) compatibility of electronic medical implants for cardiac imaging is important because MRI is frequently indicated for imaging cardiac structure and function in patients with implantable devices such as pacemakers and defibrillators. This is especially the case with patients that have congestive heart failure and atrial fibrillation. Although high quality of cardiac MRI images is required in these patients, traditional devices that are not MRI compatible perturb magnetic field and degrade image quality. In this aspect, the bioresorbable module exploits designs that minimize disturbances in the time-dependent magnetic fields associated with MRI scanning, thereby reducing distortions and shadowing artifacts in the final image and eliminating any parasitic heating from magnetically induced eddy currents. Simulations guide the selection of designs that ensure that the resonant frequencies of the bioresorbable module (about 13.56 MHz) have no overlap with the working frequencies of typical MRI scanners (64 MHz, 128 MHz, 298 MHz, and 400 MHz for 1.5T, 3T, 7T, and 9.4T MRI scanners, respectively), thereby avoiding large gradients of the magnetic field density.

Operation of the transient closed-loop system (e.g., electrical stimulation for heart pacing) in the presence of the MRI scanner (9.4T Bruker Biospec MRI system, Bruker BioSpin Corporation) confirms the ability to operate in an environment with strong magnetic field exposure. As shown in panel A of FIG. 10, the experimental procedure involved placing the bioresorbable module on a chicken thigh subcutaneously and connecting a non-resorbable LED to the exposed electrode. The skin-interfaced cardiac module was placed on the skin of the chicken with good alignment between Tx and Rx coils to wirelessly power the bioresorbable module. Blinking of the light emitting diode (LED) with a set frequency indicated the proper function of the closed-loop system (e.g., cardiac pacing). Although the Bluetooth-based data transmission was not possible in the MRI room due to magnetic shielding designed to eliminate fringing fields and RF shielding to prevent electromagnetic noise from entering or leaving the room, we observed no changes in the blinking intensity or frequency of the LED even next to the MRI. This uninterrupted operation follows from designs of the skin-interfaced cardiac module that allow it to continuously transfer power to the bioresorbable module even without a Bluetooth connection. The operation was only changed when placed at the entry to and inside the scanner bore, which is surrounded by magnetic gradient coils and RF transmit coils, due to interference with the inductive coupling between bioresorbable module and skin-interfaced cardiac module.

Removing the skin-interfaced module allows us to investigate the MRI compatibility of the bioresorbable module to mimic the situation of a patient who could temporarily function without pacing for the duration of an MRI scan. MRI imaging results indicate that the extension electrode causes less susceptibility artifacts (shadowing) compared to the Rx coil (panels B-C of FIG. 10). In practical scenarios, however, the Rx coil will be placed subcutaneously, connected to the heart using the extension electrodes, thereby minimizing the influence of this part of the device on MRI image distortion. Calculations of the gradients of the magnetic field density near bioresorbable modules on biological tissues in a 1.5T MRI scanner reveal the underlying effects. Panel D of FIG. 10 shows that the module induces a weaker disturbance to the magnetic field than commercial electrodes with similar overall sizes and geometries for both the in-plane |∇ₚB| and out-of-plane |∇_{z}B| gradient of the magnetic field density(11). In this test, the resultant temperature change at the interface with the chicken thigh after MRI scanning is far below the threshold for sensation even in the area near Rx coil due partly to the insulating effects of the b-DCPU. Full three-dimensional multi-physics modeling also shows that, at the end of a single scan for 1 s, the Mo-based electrodes of the bioresorbable module undergo heating by only 0.23° C. (FIG. 11). The resultant maximum temperature change at the interfaced heart tissue is 0.035° C., far below the threshold for tissue damage or perception of heat (The maximum change in temperature of heart tissue occurs about 0.17 s after initiating the scan). These results meet FDA requirements for an “MRI-safe” label for medical devices and therefore allow the devices to remain in the body during MRI imaging.

### Example 5

### Functional Lifetime of Bioresorbable Modules

In the exemplary example, we prepared two different devices with different constituent materials: one uses 10 μm thick Mo electrodes (i.e., bioresorbable module); the other uses tungsten-coated magnesium (W, about 700 nm thick; Mg, about 50 μm thick) electrodes as a control group, described in our previous work. The functional lifetime of the W/Mg-based device is only 4 days in the in vivo rat model. To extend this short functional lifetime, we (i) introduce a bioresorbable conductor (Mo) with a slow rate of degradation (20 nm/day), (ii) engineer a stretchable electrode structure by adopting a stretchable polymer encapsulation and exploiting serpentine-structured traces, and (iii) introduce a bioresorbable steroid eluting patch to reduce the fibrotic tissue growth at the electrode-tissue interface. As shown in panel F of FIG. 2, the W/Mg-based reference module (black circle) maintains its function up to 8 days after immersion in PBS (pH 7.4; 37° C.), corresponding to the degradation rate of Mg (about 7 μm/day). The Mo-based bioresorbable module (red square) operates for more than 30 days under the same simulated physiological conditions due to the much slower rate of degradation of Mo (about 20 nm/day). In the physiologically narrow temperature range (37-40° C.), there is no significant differences of the biodegradation behavior (FIG. 15). Fig. S12 shows optical images at various stages of dissolution of bioresorbable modules in PBS (pH 7.4; 37° C.).

The resulting degradation timeline of the single bioresorbable pacemaker (functional lifetime=32 days; full degradation time=500 days) is sufficient for safe administration electrotherapy where temporary pacing is suggested post-operatively, where the required therapeutic window (7 days) of a conventional temporary pacemaker. Within this therapeutic timeframe, it is unlikely that patients will require an additional pacemaker.

The degradation (i.e., resorption) time of the bioresorbable module may vary across patients due to intrinsic physiological differences, such as the amount of biofluid and enzymes, the rate of the recovery, level of the activity, and speed of metabolism. The functional lifetime of the bioresorbable module can be tailored by selecting the appropriate thicknesses and types of constituent materials, and external stimuli to meet the needs of the patient and their required period for the therapeutic treatment.

### Example 6

### Considerations for Heart Pacing

Electrical waveform for heart pacing: The waveform employed in the invention is a cathodic monophasic square wave pulse where the amplitude and the pulse width/duty cycle are adjusted based on excitation threshold. Compared to cathodal stimulation, anodal stimulation induces faster conduction velocities. However, anodal monophasic stimulation has higher pacing thresholds and potentially causes corrosion of the metal electrode interface. The biphasic mode of pacing represents an attractive alternative because the additional anodal component of the waveform has several key advantages: (i) increased conduction velocity compared to pure cathodal monophasic pacing, and (ii) fast and complete reactivation of the sodium current. It is desired to incorporate a circuit structure in the bioresorbable module for charge-balanced biphasic waveform to minimize damage at the tissue-electrode interface.

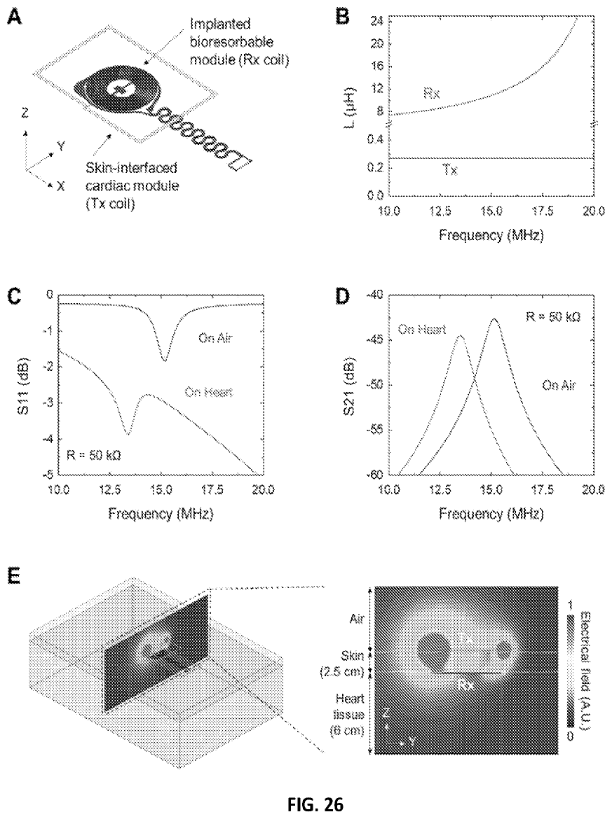

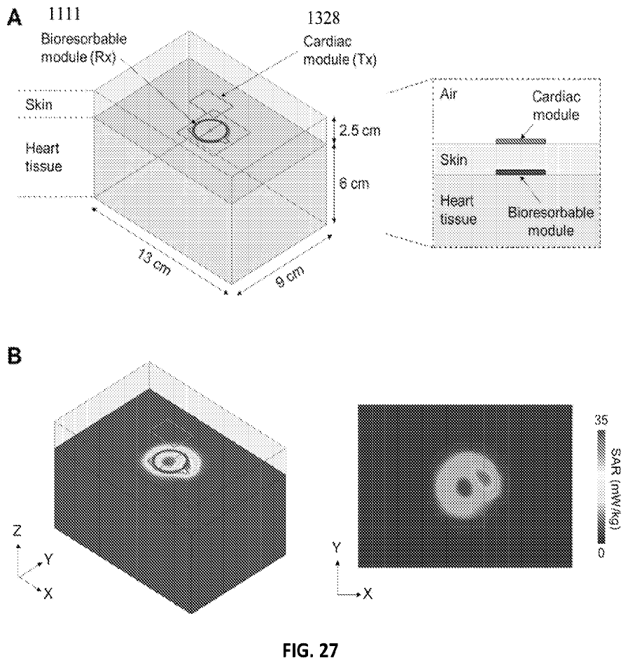

Safety issues related to the inductive coupling-based wireless power transmission for heart pacing: Electromagnetic characteristics for inductive coupling between Rx coil 1111 in the bioresorbable module 11 and Tx coil 1328 in the skin-interfaced cardiac modules 132 are demonstrated in FIG. 26. In addition, electromagnetic characteristics of the transient closed-loop system in an in vivo human heart model is conducted in FIG. 27. The results of these computational works verify that operation of the transient closed-loop system and their wireless power transmission falls within guidelines outlined by the FCC (47 CFR Part 1.1310 and 15) and the FDA.

### Example 7

### Continuous and Long-Term Pacing Capabilities

Investigations using rodent models demonstrate continuous, long-term pacing and biocompatibility.

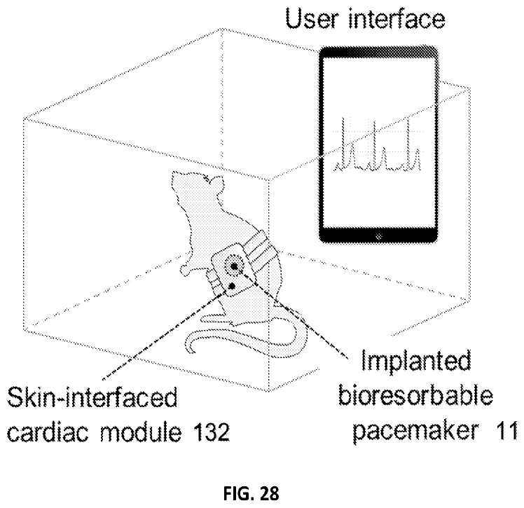

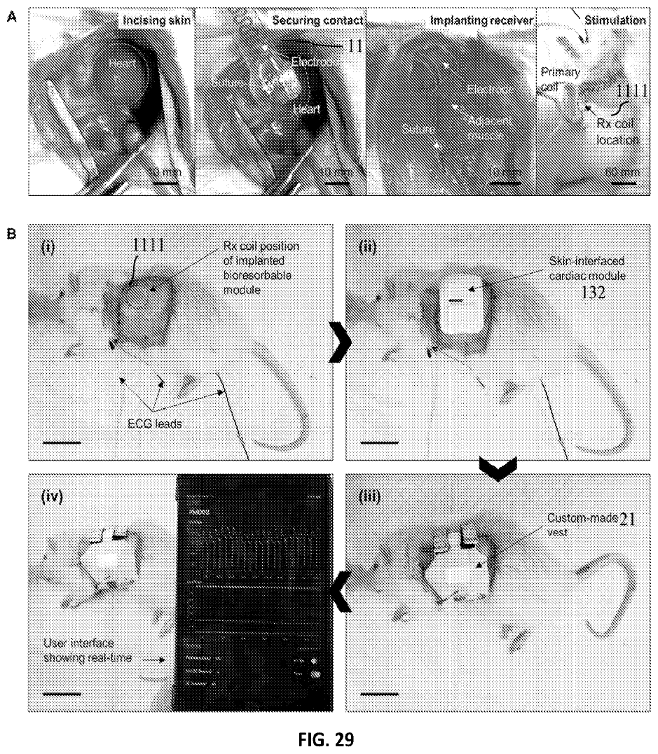

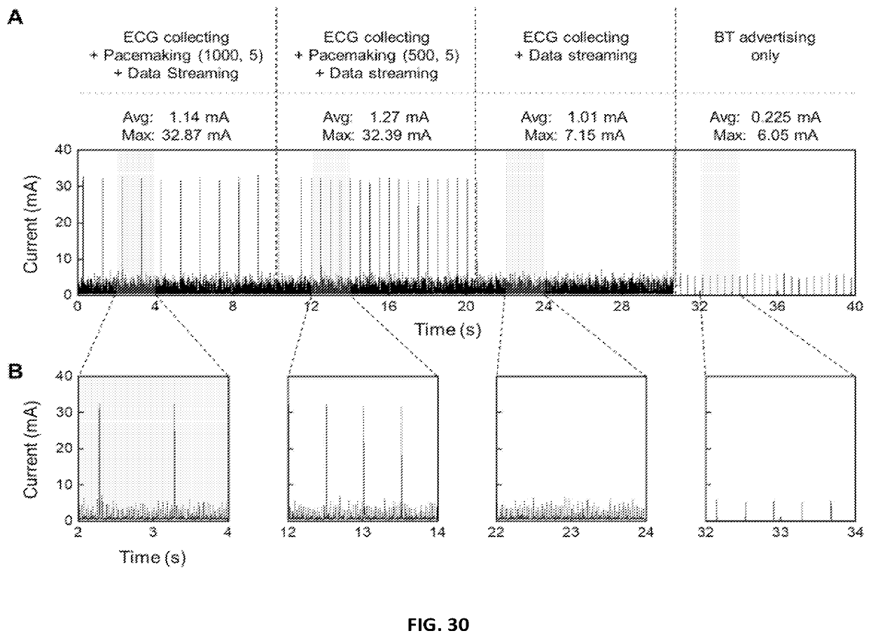

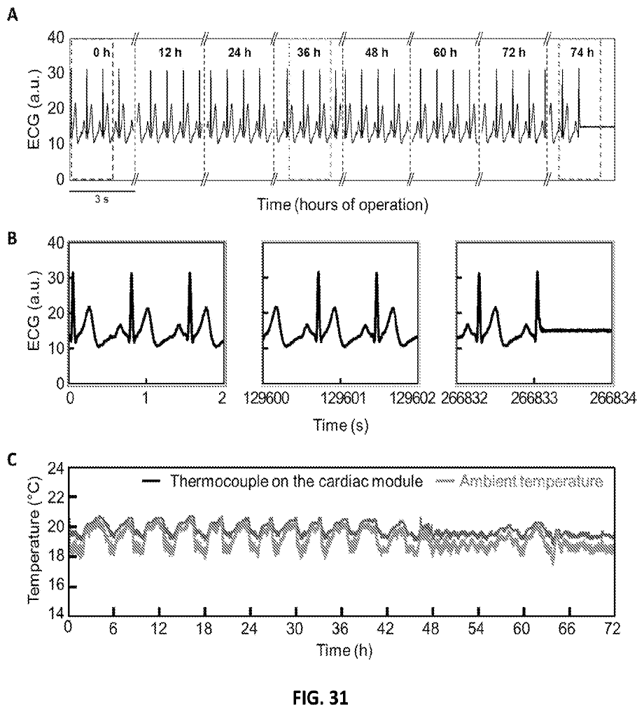

Continuous pacing capability: Cardiac bradyarrhythmias often arise following a major cardiac surgery such as coronary artery bypass grafting (CABG), septal defect repair, or valve surgery. In such cases, temporary pacemakers may be required to pace the heart continuously for up to several weeks. Demonstration of continuous pacing capabilities of the system of the invention use an in vivo rat model illustrated in FIG. 28. The animal, implanted with a bioresorbable module 11, roams tether-free in its cage while wearing a skin-interfaced cardiac module 132 that is secured under a custom vest 21 (FIG. 29). In the clinical setting, utilization of the additional vest 21 (or chest straps) is suggested to patients to prevent the falling off the skin-interfaced devices 13, especially for the cardiac module 132 since it wirelessly powers the implantable stimulator to pace the heart. A mobile application 15 continuously records ECG waveforms and determines the parameters for pacing. The power consumed by the cardiac module 132 during Bluetooth data streaming, ECG collection, RF power transmission, and bluetooth advertising appear in FIG. 30. The battery used here (80 mAh; 12 mm×25 mm×4.0 mm; 3.7V lithium polymer) can power the cardiac module 132 to continuously pace the heart (60 Hz pacing with 0.5% duty cycle signal) for more than 3 days (FIG. 31).

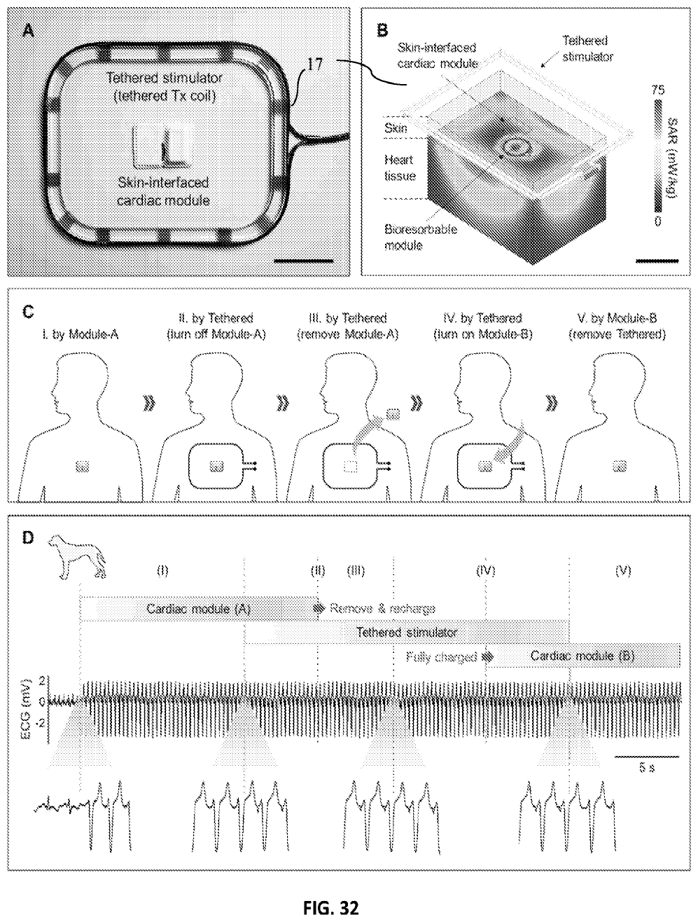

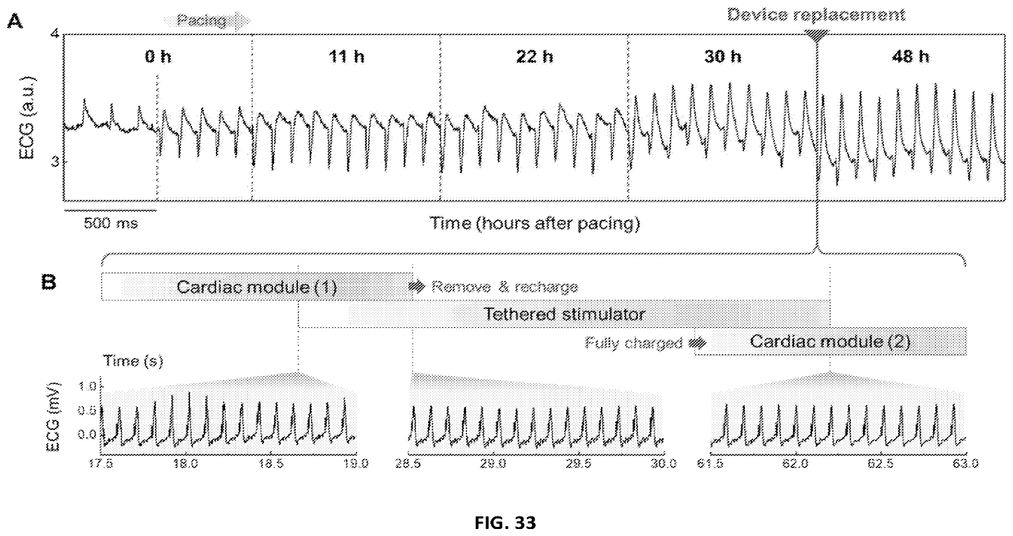

As demonstrated in an in vivo canine model (FIG. 32), upon nearing depletion of the battery, the cardiac module 132 can be rapidly exchanged with a charged unit without interrupting pacing (FIG. 32). Here, we demonstrate a scenario where the patient replaces the cardiac module 132 at the 30-hour time point. Throughout the first 30h of pacing using an in vivo rat model, the ECG indicates continuous pacing and ventricular capture. In the replacement process (panel A of FIG. 33), the rat resides in an arena surrounded by a wall-tethered Tx coil that wirelessly powers the implantable bioresorbable module 11. The ECG data in panel B of FIG. 33 show that ventricular capture is continuous throughout the entire battery exchange procedure. After attaching a cardiac module 132 with a fully charged battery, continuous pacing and ECG monitoring seamlessly resumes until 48 h. These results confirm that the transient closed-loop system can pace the heart continuously and that the skin-interfaced cardiac modules 132 can be easily recharged and replaced without interruption of life-saving pacing.

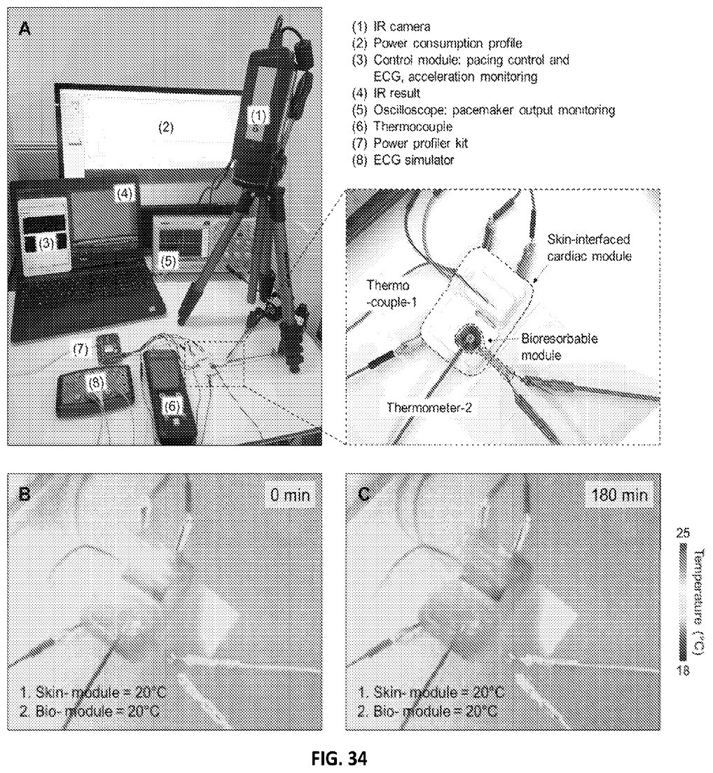

Heat development of the modules during the operation and recharging: In the invention, temperature of the devices during their standard functions (electrical stimulation for cardiac pacing) and re-charging are measured. Panel A of FIG. 34 shows the experimental setup for temperature measurements during electrical stimulation. To measure the change in the temperature, the skin-interfaced cardiac module 132 and bioresorbable module 11 are placed on a wooden table and operated continuously for 180 min. As shown in panel B of FIG. 34, there are no significant changes in the temperature for the skin-interfaced modules 13 or the bioresorbable module 11 even after 180 min continuous operation of the entire closed-loop system, due to the low power nature of the inductive coupling-based powering and optimized Bluetooth communication.

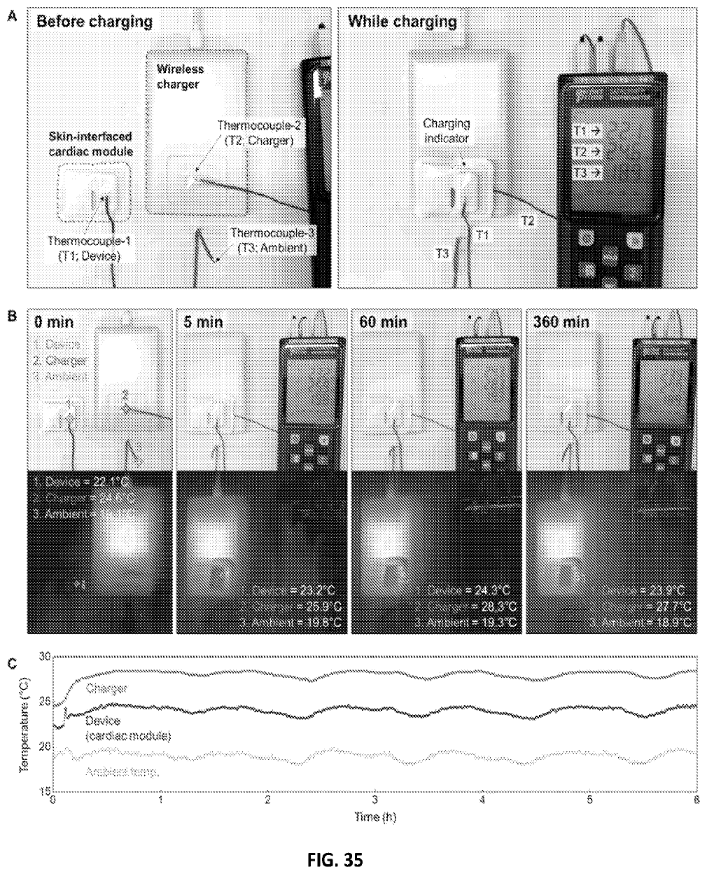

The skin-interfaced device 13 can be recharged by using the wireless charger during continuous pacing during which time the tethered-Tx coil 17 can wirelessly power the implantable bioresorbable module 11 (FIG. 33). Panels A-B of FIG. 35 show the experimental setup and the results of temperature measurements of the skin-interfaced cardiac module 132 during 6 hours of wireless charging, respectively. An IR camera monitored the temperature distribution of the system during wireless charging, and k-type thermocouples measured the local temperature of the surface of the skin-interfaced cardiac module 132 (T1; on the battery), the surface of the wireless charger (T2; on the wireless coil), and an empty region near the device as a reference (T3). The results (panel B of FIG. 35) indicate that most of the heat is generated by the wireless charger (26-28° C.) during the recharging process and conducted to the skin-interfaced device 13. Panel C of FIG. 35 shows the changes in the local temperature during 6 hours of wireless charging. Once the battery charging is completed, the skin-interfaced module 13 can be located at a different place, so that the conducted heat can be dissipated quickly (<1 min).

These results indicate that there is no adverse effect related to the accumulation of heat during device function or the battery charging process.

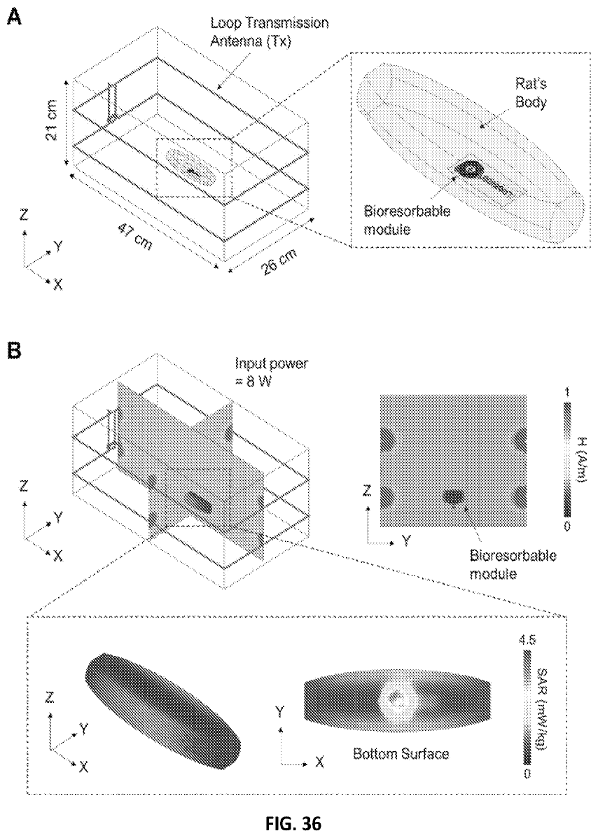

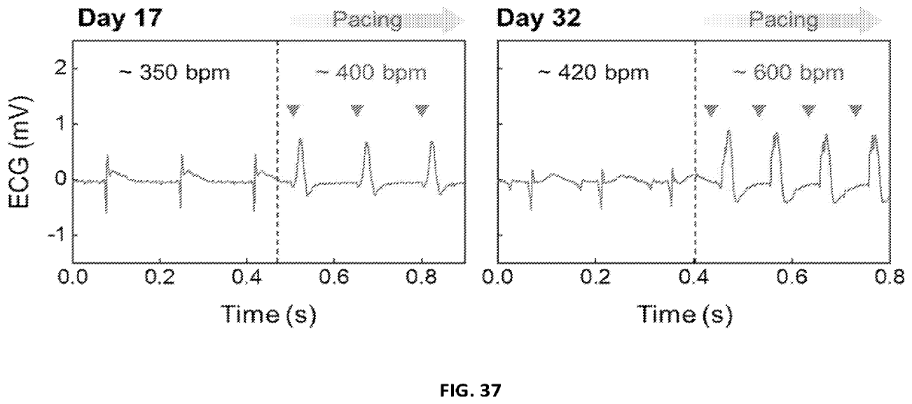

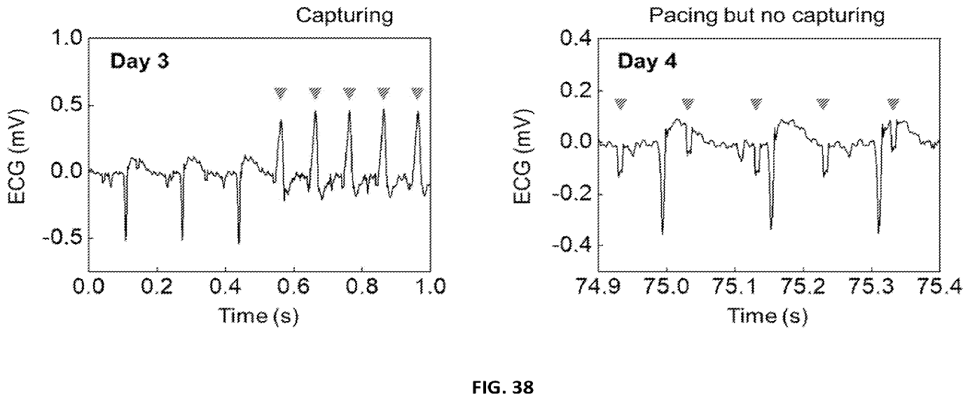

Long-term pacing capability: Longer-term studies in this in vivo rat model highlight the efficacy of the stretchable design of the bioresorbable module 11 and its steroid eluting system 1151 at the tissue-electrode interface. In these experiments, a cage-based RF powering system with Tx loop coil provides wireless operation within a region of interest (FIG. 36). Daily pacing trials demonstrate that the bioresorbable module 11 has a functional lifetime of 32 days (FIG. 37). This functional lifetime is significantly longer than that of previously reported bioresorbable electronic devices, including an earlier version of the bioresorbable temporary pacemaker (without stretchable design or steroid eluting system). Furthermore, 32 days of operation period is more than a factor of four larger than the time period of 7 days that patients typically require temporary pacemakers, consistent with safe administration of electrotherapy where temporary pacing is indicated post-operatively. Devices with other designs (non-stretchable device with steroid system; stretchable device without steroid system) remain functional for only up to 4-8 days (FIG. 38).

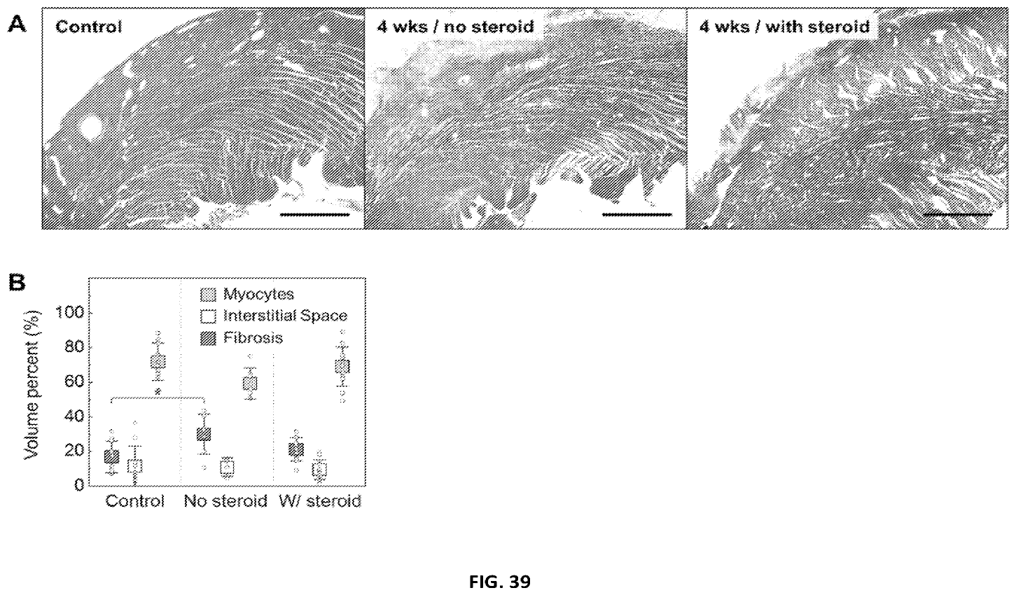

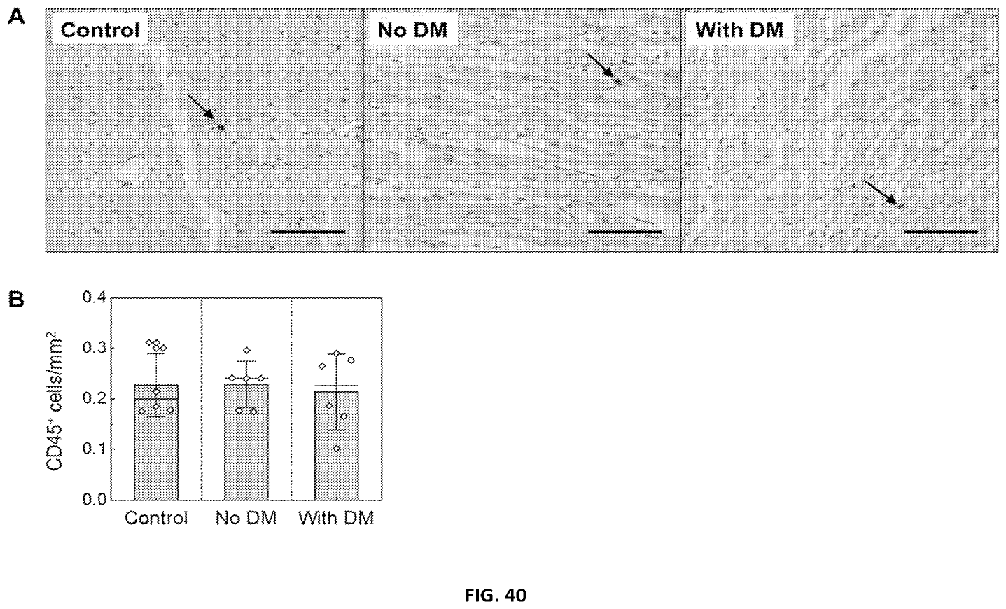

Histological and immunohistochemical evaluations: Histological and immunohistochemical evaluations of the myocardium reveal the efficacy of the steroid eluting system 1151 for long-term pacing capabilities. Masson's trichrome staining quantifies the volume of myocardium, fibrotic tissue, and interstitial space in the cardiac cross sections of rats with and without steroid eluting implanted devices near the site of device attachment (FIG. 39). Quantitative analysis shows that the steroid eluting electrode significantly attenuates fibrotic tissue growth in the transmural myocardium over 4 weeks when compared to the non-steroid-eluting case (panel B of FIG. 39). Immunohistochemical staining of the pan-leukocyte marker CD45⁺ in the same cardiac cross sections reveal the level of inflammation in hearts. The frequency of CD45⁺ cells near the site of attachment for the bioresorbable module steroid-eluting electrodes is not significantly different from animals without any pacemaker implanted (FIG. 40). These results confirm that the steroid-eluting pacemaker significantly inhibits fibrotic tissue growth at the tissue-electrode interface and does not induce significant inflammation, which contributes to the long-term stability of operation.

### Example 8

### Biocompatibility of Bioresorbable Module

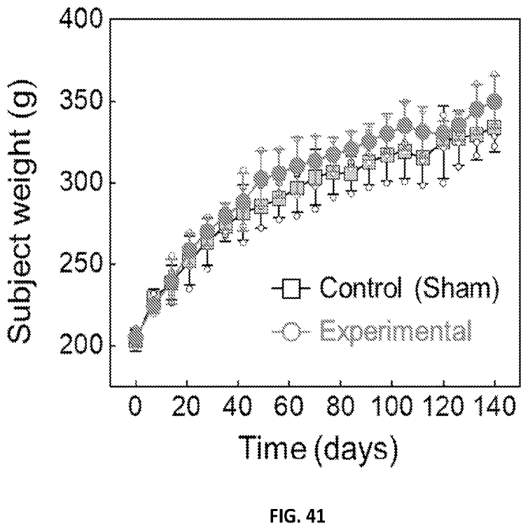

As shown in FIG. 41, tracking of the weights of the animals following implantation shows a gradual increase with time, as expected.

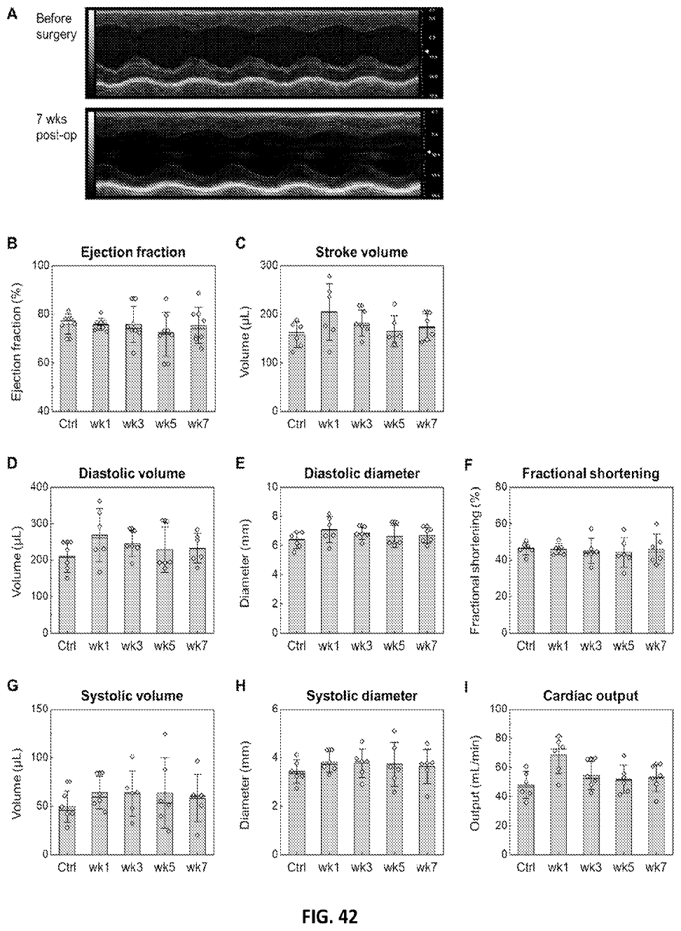

Echocardiography measurements of rats implanted with bioresorbable modules indicate that the devices have negligible effects on the mechanical function of the heart. Echocardiograms provide real-time, dynamic outlines of the walls of the heart as measurements of myocardial morphology and various hemodynamic parameters. Echocardiograms recorded 1, 3, 5, and 7 weeks after device implantation show no meaningful differences in ejection fraction or other hemodynamic parameters (diastolic volume, diastolic diameter, fractional shortening, systolic volume, systolic diameter, cardiac output) (FIG. 42) over the 7 weeks.

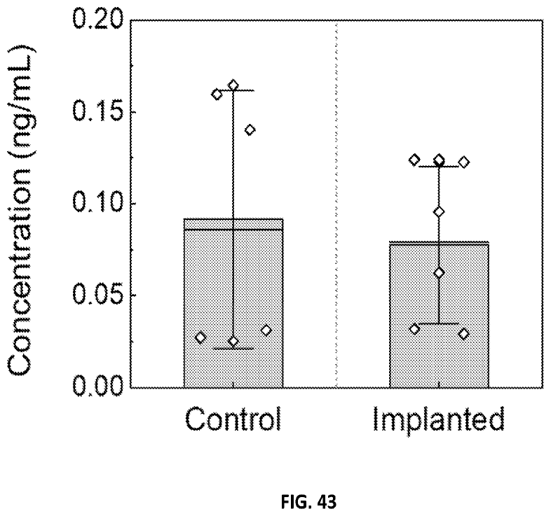

FIG. 43 shows levels of brain natriuretic peptide 45 in the myocardium which indicates that the bioresorbable module and its implantation do not induce damage to the heart. BNP 45 is a biomarker for myocardial infarction. Levels of BNP 45 rise over several weeks following surgery for cases of myocardial infarction. Enzyme-linked immunosorbent assays (ELISA) and BNP 45 show no significant differences in levels of these species between naïve rats and device-implanted rats (BNP 45: p=0.3429).

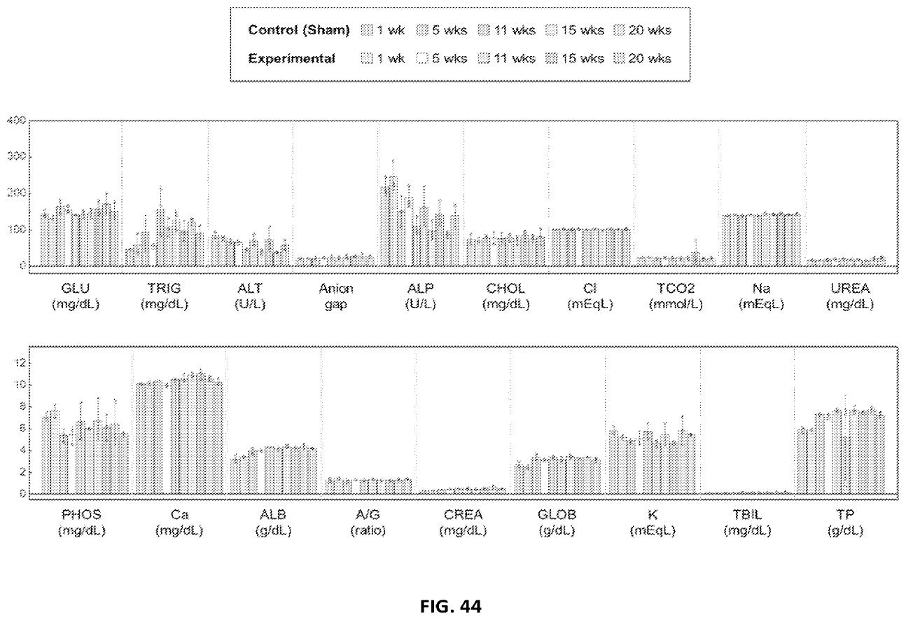

The results of serology tests provide a comprehensive understanding of the health status of rats with implanted pacemakers as the devices resorb, according to FIG. 44. Blood levels of enzymes and electrolytes, as indicators of organ-specific diseases, fall within the confidence intervals of control values. Specifically, normal levels of alanine aminotransferase, cholesterol and triglyceride, phosphorus and urea, nitrogen, calcium, albumin, and total proteins respectively indicate the absence of disorders in liver, heart, kidney, bone, and nerve tissue, as well as good overall health.

Taken together, these histologic, echocardiographic, immunohistochemical and serologic results illustrate that the implantation, operation, and resorption of the bioresorbable module do not impact natural physiology of the rodent model. Future studies will assess the overall health status and possible tissue responses to the implanted bioresorbable module in the large pre-clinical animal models.

### Example 9

### Detailed Feedback Control Process for Bradycardia Treatment

The sensors in the cardiac module monitor cardiac electrocardiograms. Real-time signal processing methods classify the bradycardia based on a heart rate value that falls below a threshold value (=lower rate limit). In this condition, the system automatically activates the bioresorbable module wirelessly, to provide electrical stimulation to the myocardium at a pre-specified rate. If the heart rate is above a certain threshold (=higher rate limit), the system remains inactive. This feedback control algorithm maintains the heart rate within a programmed range programmed range to restore normal rhythms. The pacing can also be automatically terminated after a programmed period (=pacing duration), allowing clinical operators to monitor and reassess the underlying heart rhythm of the patient between pacing treatments. These functions (lower and higher rate limit, and pacing duration), along with the various parameters associated with the pacing waveforms (pacing frequency, pulse width) can be defined manually on the user interface to address specific patient needs. In clinical practice, the pacing inhibition function is limited to brief, pre-specified times when the patient is supine to minimize risks associated with interruption of pacing.

### Example 10

### Rate-Adaptive Pacing Mechanism

Simple forms of rate-adaptive pacing in conventional, permanent devices use accelerometers to determine metabolic needs according to characteristics of thoracic motion. Although this method can be effective, it may require customized programming and threshold adjustment by a trained operator or physician for some patients. More importantly, the utility is limited when increased metabolic demand does not correlate with core motion, such as with stationary exercise.

For the demonstration of the rate-adaptive pacing function of the transient closed-loop system, physical activity was estimated from the magnitude of the triaxial accelerometer data using a developed algorithm (FIG. 50). In addition, the pacing signal was determined during a predefined window (in this case, every 10 s) using post data processing.

Adding the monitoring function of emotional stress-triggered sympathetic nerve activity into the skin-interface modules will enable rate-adaptive on-demand pacing for not only physical activity but also flight-or-flight response

### Example 11

### Strategies for Stable and Reliable Pacing

Using respiratory rate in addition to accelerometer measurements also limits inappropriate increases in pacing rate that results from external motion. For example, the sensor may detect an apparent increase in physical activity from an external source (e.g., tapping on the sensor or a bumpy car ride) with no increase in respiration rate. In these aberrant cases, the system maintains the pacing rate below a certain level (e.g., 60 or 70 bpm) since the respiratory rate does not rise significantly above resting levels (e.g., 20 rpm). In addition, Proportional-Integral-Derivative (PID) control yields a more stable and reliable pacing rate by using the error between the pacing set value and the measured value continuously against disturbances including sudden electric shocks due to large RF pulses or defibrillation processes (FIG. 52). The monitoring physician can also substitute algorithms currently used in state-of-the-art permanent pacemakers into this hardware system and make setting adjustments remotely. A cross-check validation method ensures signal quality. FIG. 53 shows the block diagram of the cross-check validation scheme for generating reliable pacing signals. The control module processes real-time HR and respiratory rate locally and performs cross-checking validation with the transmitted HR and respiratory rate values from the networked collection of skin-interfaced devices including cardiac, respiratory, and hemodynamic modules. This method prevents misguided pacing signal generation protecting against device malfunction and external hacking or spoofing.

In summary, the transient, closed-loop system that combines a time-synchronized, wireless network of soft, skin-integrated devices with an advanced bioresorbable pacemaker to control cardiac rhythms, track cardiopulmonary status, provide multi-haptic feedback, and enable transient operation with minimal patient burden. The system provides a range of robust, rate-adaptive cardiac pacing capabilities, as demonstrated in rat, canine, and human heart studies of autonomous treatment of bradycardias. The invention establishes a generalizable engineering framework for closed-loop temporary electrotherapy using wirelessly linked, body-integrated bioelectronic devices.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the invention pertains without departing from its spirit and scope. Accordingly, the scope of the invention is defined by the appended claims rather than the foregoing description and the exemplary embodiments described therein.

Some references, which may include patents, patent applications and various publications, are cited and discussed in the description of this invention. The citation and/or discussion of such references is provided merely to clarify the description of the invention and is not an admission that any such reference is “prior art” to the invention described herein. All references cited and discussed in this specification are incorporated herein by reference in their entireties and to the same extent as if each reference was individually incorporated by reference.

### LIST OF REFERENCES

[1]. A. L. Waldo, J. L. J. Wells, T. B. Cooper, W. A. MacLean, Prog. Cardiovasc. Dis. 23, 451-474 (1981).
[2]. F. W. Chapman, J. C. Newell, R. J. Roy, Ann. Biomed. Eng. 13, 359-372 (1985).
[3]. J. L. Austin, L. K. Preis, R. S. Crampton, G. A. Beller, R. P. Martin, Am. J. Cardiol. 49, 301-306 (1982).
[4]. F. J. Lumia, J. C. Rios, Chest. 64, 604-608 (1973).
[5]. K. D. Donovan, K. Y. Lee, Anaesth. Intensive Care. 13, 63-70 (1985).
[6]. M. U. Braun et al., PACE—Pacing Clin. Electrophysiol. 29, 875-879 (2006).
[7]. K. A. McLeod, Heart. 96, 1502-1508 (2010).
[8]. E. Buch, N. G. Boyle, P. H. Belott, Circulation. 123, 378-380 (2011).
[9]. R. G. Hauser, W. T. Katsiyiannis, C. C. Gornick, A. K. Almquist, L. M. Kallinen, EP Eur. 12, 395-401 (2010).
[10]. Y. S. Choi et al., Nat. Biotechnol. 39, 1228-1238 (2021).
[11]. Y. S. Choi, J. Koo, J. A. Rogers, MRS Bull. 45, 103-112 (2020).
[12]. J. Koo et al., Sci. Adv. 6, eabb1093 (2020).
[13]. S. K. Kang et al., Nature. 530, 71-76 (2016).
[14]. H. G. Mond, J. R. Helland, K. Stokes, G. A. Bornzin, R. McVenes, PACE—Pacing Clin. Electrophysiol. 37, 1232-1249 (2014).
[15]. K. B. Stokes, G. A. Bornzin, W. A. Wiebusch, K. Steinbach, Ed. (Steinkopff, Heidelberg, 1983), pp. 369-376.
[16]. Y. S. Choi et al., Nat. Commun. 11, 5990 (2020).
[17]. Y. S. Choi et al., Adv. Funct. Mater. 30, 2000941 (2020).
[18]. E. K. S. Espe et al., J Cardiovasc. Magn. Reson. 15, 82 (2013).
[19]. H. U. Chung et al., Nat. Med. 26, 418-429 (2020).
[20]. K. Lee et al., Nat. Biomed. Eng. 4, 148-158 (2020).
[21]. H. Jeong et al., Sci. Adv. 7 (2021), doi:10.1126/sciadv.abg3092.
[22]. A. Y. Rwei et al., Proc. Natl. Acad. Sci. U.S.A 117, 31674-31684 (2020).
[23]. J. Pan, W. J. Tompkins, IEEE Trans. Biomed. Eng. BME-32, 230-236 (1985).
[24]. S.-W. Hwang et al., Science. 337, 1640-1644 (2012).
[25]. E. Garcia-Izquierdo, S. Vilches, V. Castro, Circulation. 135, 711-713 (2017).
[26]. J. G. Webster, I. E. in Medicine, B. Society, Design of Cardiac Pacemakers (IEEE, 1995; https://books.google.com/books?id=W1VyQgAACAAJ), Studies in Asian Thought and Religion.
[27]. S.-K. Leung, C.-P. Lau, Cardiol. Clin. 18, 113-155 (2000).
[28]. S. GARRIGUE et al., Pacing Clin. Electrophysiol. 25, 883-887 (2002).
[29]. M. Coenen et al., EP Eur. 10, 327-333 (2008).
[30]. S. M. Won et al., Adv. Funct. Mater. 28, 1801819 (2018).
[31]. X. Yu et al., Nature. 575, 473-479 (2019).
[32]. IEEE Standard for Safety Levels with Respect to Human Exposure to Radio Frequency Electromagnetic Fields, 3 kHz to 300 GHz. IEEE Std C95.1-2005 (Revision IEEE Std C95.1-1991) (2006), pp. 1-238.
[33]. K. F. Winter et al., J. Neurosci. Methods. 79, 79-85 (1998).
[34]. H. Dinis et al., Micromachines. 8, 359 (2017).
[35]. S. F. Pichorim et al., in Proceedings of the 14th International Symposium on Biotelemetry (1998), pp. 71-77.
[36]. A. Kurs et al., Soljačid, Science, in press, doi:10.1126/science.1143254.
[37]. J. Koo et al., Nat. Med. 24, 1830-1836 (2018).
[38]. N. V Thakor et al., Am. J. Cardiol. 79, 36-43 (1997).
[39]. A. Barsheshet et al., Ann. noninvasive Electrocardiol. 17, 22-27 (2012).
[40]. A. Thakral et al., J. Appl. Physiol. 89, 1159-1164 (2000).
[41]. K. M. Kavanagh et al., Pacing Clin. Electrophysiol. 13, 1268-1276 (1990).
[42]. T. Force et al., Circulation. 82, 903-912 (1990).
[43]. P. M. Zoll et al., Circulation. 71, 937-944 (1985).
[44]. W. H. Maisel et al., Ann. Intern. Med. 135, 1061-1073 (2001).
[45]. J. Shin et al., Nat. Biomed. Eng. 3, 37-46 (2019).
[46]. S. W. Hwang et al., Nano Lett. 15, 2801-2808 (2015).
[47]. K. Liu et al., MRS Bull. 45, 96-102 (2020).
[48]. S. Dell'Orto et al., Indian Pacing Electrophysiol. J. 4, 137-145 (2004).
[49]. R. Proietti et al., Pacing Clin. Electrophysiol. 35, 990-998 (2012).
[50]. P. Palmisano et al., Eur. pacing, arrhythmias, Card. Electrophysiol. J. Work. groups Card. pacing, arrhythmias, Card. Cell. Electrophysiol. Eur. Soc. Cardiol. 14, 1038-1043 (2012).
[51]. C. Krittanawong et al., Nat. Rev. Cardiol. 18, 75-91 (2021).
[52]. E. Y. Wan et al., Cardiovasc. Digit. Heal. J. 2, 196-211 (2021).

What is claimed is:

1. A transient closed-loop system for cardiac pacing and/or defibrillator therapy for a subject, comprising:
a bioresorbable module configured to at least partially attach to an epicardial interface of the subject's heart for the cardiac pacing;
at least one skin-interfaced module configured to attach to an outer surface of the subject's skin, wherein the bioresorbable module is in wireless communication with the at least one skin-interfaced module; and
a control module in wireless communication with the at least one skin-interfaced module.

2. The system of claim 1, wherein the bioresorbable module dissolves in the subject's body after a period of time.

3. The system of claim 2, wherein the period of time is at least 10 days, 20 days or 30 days.

4. The system of claim 2, wherein the period of time is customizable.

5. The system of claim 1, wherein the control module is configured to calculate at least one regular heart rate and provide autonomous cardiac pacing to the subject according to the at least one regular heart rate.

6. The system of claim 5, wherein the at least one regular heart rate comprises a high rate limit and a low rate limit.

7. The system of claim 6, wherein when a heart rate of the subject is lower than the low rate limit, the control module activates the bioresorbable module to provide electrical stimulation to the heart at a pre-specified rate, for cardiac pacing.

8. The system of claim 6, wherein when the heart rate of the subject is higher than the high rate limit, the bioresorbable module remains inactive.

9. The system of claim 5, wherein the at least one skin-interfaced module comprises a cardiac module.

10. The system of claim 9, wherein the cardiac module is configured to operably place over skin of the subject's chest area.

11. The system of claim 9, wherein the at least one skin-interfaced module further comprises a respiration module in wireless communication with the control module.

12. The system of claim 11, wherein the respiration module is configured to operably collect physiological information of the subject and wirelessly transmit the physiological information to the control module.

13. The system of claim 12, wherein the control module operably calculates the at least one regular heart rate according to the physiological information collected by the respiration module and provides autonomous cardiac pacing to the subject according to the at least one regular heart rate.

14. The system of claim 9, wherein the at least one skin-interfaced module further comprises a hemodynamic module in wireless communication with the control module.

15. The system of claim 14, wherein the hemodynamic module is configured to operably collect physiological information of the subject and wirelessly transmit the physiological information to the control module.

16. The system of claim 15, wherein the control module operably calculates the at least one regular heart rate according to the physiological information collected by the hemodynamic module and provides autonomous cardiac pacing to the subject according to the at least one regular heart rate.

17. The system of claim 9, wherein the at least one skin-interfaced module further comprises a haptic module in wireless communication with the control module.

18. The system of claim 17, wherein the haptic module in configured to operably receive tactile information from the control module.

19. The system of claim 18, wherein the haptic module operably provides at least one pattern of vibro-tactile according to the tactile information received from the control module.

20. The system of claim 9, wherein the bioresorbable module comprises:
a power harvester configured to operably receive power delivery from the cardiac module;
at least one stimulation electrode configured to operably deliver stimuli to the epicardial interface of the subject's heart for the cardiac pacing; and
a stretchable interconnect connecting the power harvester and the stimulation electrode.

21. The system of claim 20, wherein the bioresorbable module is encapsulated by bioresorbable dynamic covalent polyurethane (b-DCPU), polylactic acid (PLA), polyglycolic acid (PGA), polyglycolide (PGL), polycaprolactone (PCL), poly(glycerol sebacate) (PGS), poly(octamethylenemaleate (anhydride) citrate)) (POMaC), poly(1,8-octanediol-co-citric acid) (POC), poly(butylene succinate) (PBS), poly(butylene adipate) (PBA), ureidopyrimidinone (Upy), poly(sebacoyl diglyceride) (PSeD-U), and/or Polybuthanedithiol 1,3,5-triallyl-1,3,5-triazine-2,4,6(1H,3H,5H)-trione pentenoic anhydride (PBTPA).

22. The system of claim 20, wherein the power harvester comprises at least one receiver (Rx) coil.

23. The system of claim 22, wherein the cardiac module comprises at least one transmission (Tx) coil.

24. The system of claim 23, wherein the Rx coil of the power harvester is wirelessly coupled to the Tx coil of the cardiac module via magnetic induction for receiving power delivery from the cardiac module.

25. The system of claim 23, wherein the stimulation electrode of the bioresorbable module is operably attached to the epicardial interface of the subject's heart, and the Rx coil of the power harvester is operably placed subcutaneously and in vicinity to the cardiac module.

26. The system of claim 23, wherein the Rx coil at least partially overlaps with the Tx coil and is placed within 25 mm of the Tx coil.

27. The system of claim 23, wherein the Rx coil operably receives the power delivery from a tethered wireless charger when the cardiac module is removed from the subject.

28. The system of claim 22, wherein the cardiac module comprises:
a transmission (Tx) coil configured to deliver power to the bioresorbable module; and
a wireless charging coil configured to receive power delivery from an external power source.

29. The system of claim 28, wherein the cardiac module further comprises a Bluetooth low energy (BLE) system-on-chip (SoC), an ECG analog front end (AFE), and/or an RF power amplifier.

30. The system of claim 20, wherein the stimulation electrode comprises an electrode that is dissolvable.

31. The system of claim 30, wherein the electrode operates for more than 30 days before being dissolved.

32. The system of claim 31, wherein the electrode is formed of a bioresorbable conductor including molybdenum (Mo), zinc (Zn), iron (Fe), tungsten (W), magnesium (Mg), and/or AZ31B (3 wt % Al and 1 wt % Zn) Mg alloy.

33. The system of claim 30, wherein the stimulation electrode further comprises a bioresorbable steroid eluting patch.

34. The system of claim 33, wherein the bioresorbable steroid eluting patch is configured to operably reduce fibrotic tissue growth at an interface between the bioresorbable module and heart tissue.

35. The system of claim 9, wherein the cardiac module operably receives pacing information from the control module regarding the cardiac pacing of the subject's heart.

36. The system of claim 35, wherein the cardiac module operably delivers the pacing information to the bioresorbable module so as to control the cardiac pacing.

37. The system of claim 1, wherein the bioresorbable module operably provides a charge-balanced biphasic waveform.

38. The system of claim 1, wherein the bioresorbable module is stretchable, twistable, and bendable.

39. The system of claim 1, wherein the skin-interfaced module is stretchable, pristinable, and bendable.

40. The system of claim 1, wherein the skin-interfaced module is peelable from the skin of the subject.

41. The system of claim 1, wherein the control module comprises a hand-held terminal.

42. The system of claim 1, wherein the control module has an interactive interface for receiving and displaying information.

43. The system of claim 1, wherein the system operably provides the cardiac pacing for treatment of bradycardia.

44. The system of claim 1, wherein the system operably detects a heart rate of the subject and determines a heart condition based on the heart rate, and initiates the cardiac pacing without the subject's intervention.

45. The system of claim 1, wherein the system is MRI safe.

46. A transient closed-loop system for cardiac pacing and/or defibrillator therapy for a subject, comprising:
a bioresorbable module for cardiac pacing and/or defibrillator therapy; and
a network of skin-integrated modules coupled with the bioresorbable module to control cardiac rhythms, track cardiopulmonary status, provide multi-haptic feedback, and enable transient operation with minimal patient burden.

47. The system of claim 46, wherein the bioresorbable module is configured to wirelessly receive power by inductive coupling to pace the subject's heart through an epicardial interface of the heart for epicardial pacing.

48. The system of claim 47, wherein the bioresorbable module comprises:
a bioresorbable, stretchable epicardial pacemaker operably attached to the epicardial interface;
a bioresorbable steroid-eluting patch coupled to the pacemaker and configured to minimize local inflammation and fibrosis; and
a bioresorbable power harvester coupled to the pacemaker to power the pacemaker.

49. The system of claim 48, wherein the pacemaker comprises at least one stimulation electrode connected to the power harvester via stretchable interconnects and configured to operably deliver stimuli to the epicardial interface of the subject's heart for the cardiac pacing.

50. The system of claim 49, wherein the power harvester comprises an antenna for delivering power to the pacemaker, wherein the antenna comprises a loop antenna having at least one coil.

51. The system of claim 50, wherein the power harvester further comprises at least one PIN diode electrically coupled between the antenna and the pacemaker.

52. The system of claim 50, wherein the stimulation electrode, the interconnects and the antenna are formed of a bioresorbable conductor including molybdenum (Mo), zinc (Zn), iron (Fe), tungsten (W), magnesium (Mg), and/or AZ31B (3 wt % Al and 1 wt % Zn) Mg alloy.

53. The system of claim 48, wherein the bioresorbable module further comprises top and bottom encapsulating layers formed of a bioresorbable dynamic covalent polyurethane (b-DCPU) that define a mechanically stretchable structure sealed by thermally activated dynamic bond exchange reactions.

54. The system of claim 48, wherein the bioresorbable module is a fully implantable, bioresorbable module.

55. The system of claim 48, wherein the bioresorbable module dissolves in the subject's body after a period of time.

56. The system of claim 48, wherein the network of skin-integrated modules comprises:
a set of flexible, skin-interfaced sensors placed on various locations of the body and configured to capture physiological monitoring of the subject, wherein the physiological information comprises electrocardiograms (ECGs), heart rate (HR), respiratory information, physical activity, and/or cerebral hemodynamics;
a radiofrequency (RF) module configured to wirelessly transfer the power from an external power source to the power harvester; and
a flexible, skin-interfaced haptic actuator configured to communicate via mechanical vibrations.

57. The system of claim 56, wherein the set of flexible, skin-interfaced sensors comprises at least one respiration module, and/or at least one hemodynamic module.

58. The system of claim 56, wherein the radiofrequency (RF) module comprises a cardiac module comprising:
a wireless charging unit configured to receive the power charged from an external power source; and
a transmission (Tx) coil configured to wirelessly transmit the power to the power harvester.

59. The system of claim 58, further comprising a control module in wireless communication with the network of skin-interfaced modules for receiving information from the skin-interfaced modules and controlling the skin-interfaced modules.

60. The system of claim 59, wherein the control module comprises a portable device with a software application for real-time visualization, storage, and analysis of data for automated adaptive control.

61. The system of claim 59, wherein the skin-interfaced haptic actuator comprises a haptic module configured to wirelessly receive tactile information, and provide at least one pattern of vibro-tactile according to the tactile information.

62. The system of claim 61, wherein the control module operably calculates the at least one regular heart rate according to the physiological information collected by the respiration module and the hemodynamic module, and provides autonomous and wireless pacing therapy to the subject according to the at least one regular heart rate.

63. The system of claim 62, wherein the at least one regular heart rate comprises a high rate limit and a low rate limit.

64. The system of claim 63, wherein when a heart rate of the subject is lower than the low rate limit, the control module activates the bioresorbable module to provide electrical stimulation to the heart at a pre-specified rate, for autonomous and wireless pacing therapy.

65. The system of claim 63, wherein when the heart rate of the subject is higher than the high rate limit, the bioresorbable module remains inactive.

66. The system of claim 62, wherein the control module processes real-time HR and respiratory rate locally and performs cross-checking validation with the transmitted HR and respiratory rate values from networked collection of the skin-interfaced modules.

67. A method for installing a transient closed-loop system for cardiac pacing and/or defibrillator therapy for a subject, comprising:
coupling at least a part of a bioresorable module to an epicardial interface of the subject's heart for the cardiac pacing; and
attaching at least one skin-interfaced module to an outer surface of the subject's skin,
wherein the bioresorbable module is in wireless communication with the at least one skin-interfaced module; and a control module is in wireless communication with the at least one skin-interfaced module.

68. The method of claim 67, wherein the at least one skin-interfaced module comprising a cardiac module.

69. The method of claim 68, wherein the cardiac module is placed over skin of the subject's chest area.

70. The method of claim 67, wherein the at least one skin-interfaced module further comprises a hemodynamic module in wireless communication with the control module; and wherein the method further comprises attaching the hemodynamic module to the skin outer surface of the subject.

71. The method of claim 70, wherein the at least one skin-interfaced module further comprises a respiration module in wireless communication with the control module; and wherein the method further comprises attaching the respiration module to the skin outer surface of the subject.

72. The method of claim 71, wherein the at least one skin-interfaced module further comprises a haptic module in wireless communication with the control module; and wherein the method further comprises attaching the haptic module to the skin outer surface of the subject.

73. The method of claim 67, wherein the skin-interfaced module is peelable from the skin of the subject.

74. The method of claim 67, wherein the bioresorbable module dissolves after a period of time.

75. The method of claim 74, wherein the period of time is at least 10 days, 20 days, 30 days, or customizable.

76. The method of claim 67, wherein the bioresorbable module is encapsulated by bioresorbable dynamic covalent polyurethane (b-DCPU), polylactic acid (PLA), polyglycolic acid (PGA), polyglycolide (PGL), polycaprolactone (PCL), poly(glycerol sebacate) (PGS), poly(octamethylenemaleate (anhydride) citrate)) (POMaC), poly(1,8-octanediol-co-citric acid) (POC), poly(butylene succinate) (PBS), poly(butylene adipate) (PBA), ureidopyrimidinone (Upy), poly(sebacoyl diglyceride) (PSeD-U), and/or Polybuthanedithiol 1,3,5-triallyl-1,3,5-triazine-2,4,6(1H,3H,5H)-trione pentenoic anhydride (PBTPA).

77. The method of claim 67, wherein the bioresorbable module comprises:
a power harvester configured to operably receive power delivery from the cardiac module;
a stimulation electrode configured to operably deliver stimulus to the epicardial interface of the subject's heart for the cardiac pacing; and
a stretchable interconnect connecting the power harvester and the stimulation electrode.

78. The method of claim 77, wherein the power harvester comprises at least one receiver (Rx) coil.

79. The method of claim 78, wherein the cardiac module comprises:
a transmission (Tx) coil for delivering power to the bioresorbable module; and a wireless charging coil configured to receive power delivery from an external power source.

80. The method of claim 79, further comprising wirelessly coupling the Rx coil of the power harvester to the Tx coil of the cardiac module such that the bioresorbable module receives power delivery from the Tx coil of the cardiac module via magnetic induction.

81. The method of claim 79, wherein the stretchable electrode of the bioresorbable module is attached to a surface area of the subject, and the Rx coil of the power harvester is placed subcutaneously and in vicinity to the cardiac module.

82. The method of claim 79, wherein the Rx coil of the power harvester receives power delivery from a tethered wireless charger when the cardiac module is removed from the subject.

83. The method of claim 77, wherein the stimulation electrode comprises an electrode that is dissolvable.

84. The method of claim 83, wherein the electrode is formed of a bioresorbable conductor including molybdenum (Mo), zinc (Zn), iron (Fe), tungsten (W), magnesium (Mg), and/or AZ31B (3 wt % Al and 1 wt % Zn) Mg alloy.

85. The method of claim 83, wherein the stimulation electrode further comprising a bioresorbable steroid eluting patch.

86. The method of claim 85, wherein the bioresorbable steroid eluting patch is configured to operably reduce the fibrotic tissue growth at the interface between the bioresorbable module and epicardial interface.

87. The method of claim 67, wherein the control module comprises a hand-held terminal.

88. The method of claim 67, wherein the control module has an interactive interface for receiving and displaying information.

89. A method of cardiac pacing and/or defibrillator therapy for a subject with a transient closed-loop system having a bioresorable module, at least one skin-interfaced module and a control module, comprising:
wirelessly transmitting at least one parameter of cardiac pacing from the control module to the at least one skin-interfaced module;
wirelessly transmitting the at least one parameter from the at least one skin-interfaced module to the bioresobable module; and
pacing the subject's heart by the bioresorbable module according to the at least one parameter.

90. The method of claim 89, wherein the at least one skin-interfaced module comprising a cardiac module placed over skin of the subject's chest area.

91. The method of claim 90, wherein the at least one skin-interfaced module further comprising a respiration module in wireless communication with the control module.

92. The method of claim 91, wherein the at least one skin-interfaced module further comprising a hemodynamic module in wireless communication with the control module.

93. The method of claim 92, wherein the at least one skin-interfaced module further comprising a haptic module in wireless communication with the control module.

94. The method of claim 93, further comprising transmitting haptic information to the haptic module by the control module.

95. The method of claim 94, wherein the haptic information comprises at least one pattern of vibro-tactile.

96. The method of claim 95, wherein the haptic module vibrates according to the pattern of vibro-tactile received.

97. The method of claim 93, wherein the wireless communication between the control module and the cardiac module is via a Bluetooth low energy (BLE) protocol.

98. The method of claim 93, wherein the wireless communication between the cardiac module and the bioresorbable module is via at least one of a Bluetooth low energy (BLE) protocol and a near field communication (NFC) protocol.

99. The method of claim 93, further comprising:
collecting hemodynamic physiological information of the subject by the hemodynamic module; and
wirelessly transmitting the hemodynamic physiological information to the control module.

100. The method of claim 99, further comprising:
collecting respiration physiological information of the subject by the respiration module; and
wirelessly transmitting the respiration physiological information to the control module.

101. The method of claim 100, further comprising:
calculating at least one regular heart rate by the control module based on the physiological information received;
adjusting the at least one parameter for cardiac pacing by the control unit based on the physiological information; and
providing the at least one parameter to the cardiac module by the control unit.

102. The method of claim 101, further comprising:
wirelessly transmitting the at least one parameter from the cardiac module to the bioresobable module; and
pacing the subject's heart by the bioresorbable module according to the at least one parameter.

103. The method of claim 102, wherein the at least one regular heart rate comprising a high rate limit and a low rate limit.

104. The method of claim 103, wherein when a heart rate of the subject detected by the cardiac module is lower than the low rate limits, the system activates the bioresorbable module, to provide electrical stimulation to the heart at a pre-specified rate.

105. The method of claim 103, wherein when the heart rate of the subject detected by the system is higher than the high rate limits, the bioresorbable module remains inactive.

106. The method of claim 100, wherein the control module calculates at least one regular heart rate according to the respiration and hemodynamic information collected by the respiration module and the hemodynamic module and provides autonomous cardiac pacing to the subject according to the at least one regular heart rate.

107. The method of claim 106, further comprising initiating the cardiac pacing without the subject's intervention.

108. The method of claim 100, wherein the control module processes real-time HR and respiratory rate locally and performs cross-checking validation with the transmitted HR and respiratory rate values from networked collection of skin-interfaced modules including the cardiac, respiratory, and hemodynamic modules.
